SEQUENCE LISTING (22 sequences)

SEQ ID NO: 1 - DNA, 564 bp, Bacillus subtilis
atgttaaataaaatcgcagagttaattaatggagacggagaattaaaaaacgcaaaaatccaccattctctttatgcaaagttagaagcagcagcaacatctgaaaatgttgatactgatcattacataaaccatcttttaaacgaaacaataatcaggtcagaaaatgaacagatgcaagaagaaaagaaaatcgagatccataaaaacgcaaagcctaaatataaaaaattagacacatatttggacgcaactttaaaccgtaatgttttaattggtatcaccaatgaattagctggaaaagaaaccggacaagacatctatacactcactgattatttcattgattatccagcttgcagcctgtggttgaatttaactcgtcatggggaacaaagaccaattcgatttaacaacataaaagacattgagtttatagaattacctgaaaatcgtttgaaggtttgggtctacatggtggataagacttggcgatttgagatcagctatgatcctgcaattcaaaaaatcacaaatgaactgaccagtaagattagggtttaa

SEQ ID NO: 2 - protein, 181 aa, Bacillus subtilis
MKRKLLSSLAISALSLGLLVSAPTASFAAESTSTKAHTESTMRTQSTASLFATITGASKTEWSFSDIELTYRPNTLLSLGVMEFTLPSGFTANTKDTLNGNALRTTQILNNGKTVRVPLALDLLGAGEFKLKLNNKTLPAAGTYTFRAENKSLSIGNKFYAEASIDVAKRSTPPTQPCGCN

SEQ ID NO: 3 - protein, 306 aa, Bacillus subtilis
MRQGLMAAVLFATFALTGCGTDSAGKSADQQLQVTATTSQIADAAENIGGKHVKVTSLMGPGVDPHLYKASQGDTKKLMSADVVLYSGLHLEGKMEDVLQKIGEQKQSAAVAEAIPKNKLIPAGEGKTFDPHVWFSIPLWIYAVDEIEAQFSKAMPQHADAFRKNAKEYKEDLQYLDKWSRKEIAHIPEKSRVLVTAHDAFAYFGNEYGFKVKGLQGLSTDSDYGLRDVQELVDLLTEKQIKAVFVESSVSEKSINAVVEGAKEKGHTVTIGGQLYSDAMGEKGTKEGTYEGMFRHNINTITKALK

SEQ ID NO: 4 - protein, 242 aa, Bacillus subtilis
MPYLKRVLLLLVTGLFMSLFAVTATASAQTGGSFFDPFNGYNSGFWQKADGYSNGNMFNCTWRANNVSMTSLGEMRLALTSPAYNKFDCGENRSVQTYGYGLYEVRMKPAKNTGIVSSFFTYTGPTDGTPWDEIDIEFLGKDTTKVQFNYYTNGAGNHEKIVDLGFDAANAYHTYAFDWQPNSIKWYVDGQLKHTATNQIPTTPGKIMMNLWNGTGVDEWLGSYNGVNPLYAHYDWVRYTKK

SEQ ID NO: 5 - protein, 521 aa, Bacillus subtilis
MGLGKKLSVAVAASFMSLSISLPGVQAAEGHQLKENQTNFLSKNAIAQSELSAPNDKAVKQFLKKNSNIFKGDPSKRLKLVESTTDALGYKHFRYAPVVNGVPIKDSQVIVHVDKSDNVYAVNGELHNQSAAKTDNSQKVSSEKALALAFKAIGKSPDAVSNGAAKNSNKAELKAIETKDGSYRLAYDVTIRYVEPEPANWEVLVDAETGSILKQQNKVEHAAATGSGTTLKGATVPLNISYEGGKYVLRDLSKPTGTQIITYDLQNRQSRLPGTLVSSTTKTFTSSSQRAAVDAHYNLGKVYDYFYSNFKRNSYDNKGSKIVSSVHYGTQYNNAAWTGDQMIYGDGDGSFFSPLSGSLDVTAHEMTHGVTQETANLIYENQPGALNESFSDVFGYFNDTEDWDIGEDITVSQPALRSLSNPTKYNQPDNYANYRNLPNTDEGDYGGVHTNSGIPNKAAYNTITKLGVSKSQQIYYRALTTYLTPSSTFKDAKAALIQSARDLYGSTDAAKVEAAWNAVGL

SEQ ID NO: 6 - protein, 381 aa, Bacillus subtilis
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVAGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 7 - protein, 255 aa, Bacillus subtilis
MVKIFIDPGHGGSDPGATGNGLQEKTLTLQIALALRTILTNEYEGVSLLLSRTSDQYVSLNDRTNAANNWGADFFLSIHVNSGGGTGFESYIYPDVGAPTTTYQSTIHSEVIQAVDFADRGKKTANFHVLRESAMPALLTENGFIDTVSDANKLKTSSFIQSLARGHANGLEQAFNLKKTSSSGLYKVQIGAFKVKANADSLASNAEAKGFDSIVLLKDGLYKVQIGAFSSKDNADTLAARAKNAGFDAIVILES

SEQ ID NO: 8 - protein, 213 aa, Bacillus subtilis
MFKFKKNFLVGLSAALMSISLFSATASAASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 9 - protein, 664 aa, Bacillus subtilis
MPQENNTFYITTPIYYPSGKLHIGHAYTTVAGDAMARYKRLKGFDVRYLTGTDEHGQKIQQKAEQENITPQEYVDRAAADIQKLWKQLEISNDDFIRTTEKRHKVVIEKVFQKLLDNGDIYLDEYEGWYSIPDETFYTETQLVDIERNEKGEVIGGKSPDSGHPVELIKEESYFFRMGKYADRLLKYYEENPTFIQPESRKNEMINNFIKPGLEDLAVSRTTFDWGVKVPENPKHVVYVWIDALFNYLTALGYDTENDELYQKYWPADVHLVGKEIVRFHTIYWPIMLMALDLPLPKQVFAHGWLLMKDGKMSKSKGNVVDPVTLIERYGLDELRYYLLREVPFGSDGVFTPEGFVERINYDLANDLGNLLNRTVAMINKYFDGQIGSYKGAVTEFDHTLTSVAEETVKAYEKAMENMEFSVALSTLWQLISRTNKYIDETAPWVLAKDPAKEEELRSVMYHLAESLRISAVLLQPFLTKTPEKMFEQLGITDESLKAWDSITAFGQLKDTKVQKGEPLFPRLEAEEEIAYIKGKMQGSAPAKEETKEEEPQEVDRLPEITIDQFMDVELRVAEVIEAEPVKKADRLLKLQLDLGFEKRQVVSGIAKHYTPEELVGKKLVCVTNLKPVKLRGELSQGMILAGEADGVLKVVSIDQSLPKGTRIK

SEQ ID NO: 10 - protein, 5043 aa, Bacillus spp.
MRNNDNIRILTNPSVSHGEPLHISEKQPATIPEVLYRTATELGDTKGIIYLQPDGTEVYQSYRRLWDDGLRIAKGLRQSGLKAKQSVILQLGDNSQLLPAFWGCVLTGVVPAPLAVPPTYAESSSGTQKLKDAWTLLDKPAVITDRGMHQEMLDWAKEQGLEGFRAIIVEDLLSAEADTDWHQSSPEDLALLLLTSGSTGTPKAVMLNHRNIMSMVKGIIQMQGFTREDITFNWMPFDHVGGIGMLHLRDVYLGCQEINVSSETILMEPLKWLDWIDHYRASVTWAPNFAFGLVTDFAEEIKDKKWDLSSMRYMLNGGEAMVAKVGRRILELLEPHGLPADAIRPAWGMSETSSGVIFSHEFTRAGTSDDDHFVEIGSPIPGFSMRIVNDHNELVEEGEIGRFQVSGLSVTSGYYQRPDLNESVFTEDGWFETGDLGFLRNGRLTITGRTKDAIIINGINYYSHAIESAVEELPEIETSYTAACAVRLGQNSTDQLAIFFVTSAKLNDEQMSQLLRNIQSHVSQVIGVTPEYLLPVQKEEIPKTAIGKIQRTQLKTSFENGEFDHLLHKPNRMNDAVQDEGIQQADQVKRVREEIQKHLLTCLTEELHVSHDWVEPNANIQSLGVNSIKMMKLIRSIEKNYHIKLTAREIHQYPTIERLASYLSEHEDLSSLSADKKGTDTYKTEPERSQATFQPLSEVQKGLWTLQKMSPEKSAYHVPLCFKFSSGLHHETFQQAFGLVLNQHPILKHVIQEKDGVPFLKNEPALSIEIKTENISSLKESDIPAFLRKKVKEPYVKENSPLVRVMSFSRSEQEHFLLVVIHHLIFDGVSSVTFIRSLFDTYQLLLKGQQPEKAVSPAIYHDFAAWEKNMLAGKDGVKHRTYWQKQLSGTLPNLQLPNVSASSVDSQFREDTYTRRLSSGFMNQVRTFAKEHSVNVTTVFLSCYMMLLGRYTGQKEQIVGMPAMVRPEERFDDAIGHFLNMLPIRSELNPADTFSSFISKLQLTILDGLDHAAYPFPKMVRDLNIPRSQAGSPVFQTAFFYQNFLQSGSYQSLLSRYADFFSVDFVEYIHQEGEYELVFELWETEEKMELNIKYNTGLFDAASISAMFDHFVYVTEQAMLNPSQPLKEYSLLPEAEKQMILKTWNATGKTYPYITFHELFEQQAKKTPDRAAVSYEGQTLTYRELDEKSTQLAIYLQAHGVGPDRLAGIYVDRSLDMLVGLLAILKAGGAYVPLDPSYPAERLEYMLEDSEVFITLTTSELVNTLSWNGVTTALLDQDWDEIAQTASDRKVLTRTVTPENLAYVIYTSGSTGKPKGVMIPHKALTNFLVSMGETPGLTAEDKMLAVTTYCFDIAALELFLPLIKGAHCYICQTEHTKDVEKLKRDIRAIKPTVMQATPATWKMLFYSGWENEESVKILCGGEALPETLKRYFLDTGSEAWNMFGPTETTIWSAVQRINVECSHATIGRPIANTQIYITDSQLAPVPAGVPGELCIAGDGVAKGYYKKEELTDSRFIDNPFEPGSKLYRTGDMARWLTGGRIEYIGRIDNQVKIRGFRIELGDIESRLSEHPGILECVVVADMDNLAAYYTAKHANASLTARELRHFVKNALPAYMVPSYFIQLDHMPLTPNGKIDRNSLKNIDLSGEQLKQRQTSPKNIQDTVFTIWQEVLKTSDIEWDDGFFDVGGDSLLAVTVADRIKHELSCEFSVTDLFEYSTIKNISQYITEQRMGDASDHIPTDPAAHIEDQSTEMSDLPDYYDDSVAIIGISCEFPGAKNHDEFWENLRDGKESIAFFNKEELQRFGISKEIAENADYVPAKASIDGKDRFDPSFFQISPKDAEFMDPQLRMLLTHSWKAIEDAGYAARQIPQTSVFMSASNNSYRALLPSDTTESLETPDGYVSWVLAQSGTIPTMISHKLGLRGPSYFVHANCSSSLIGLHSAYKSLLSGESDYALVGGATLHTESNIGYVHQPGLNFSSDGHIKAFDASADGMIGGEGVAVVLLKKAADAVKDGDHIYALLRGIGVNNDGADKVGFYAPSVKGQADVVQQVMNQTKVQPESICYVEAHGTGTKLGDPIELAALTNVYRQYTNKTQFCGIGSVKTNIGHLDTAAGLAGCIKVVMSLYHQELAPSVNYKEPNPNTDLASSPFYVVDQKKTLSREIKTHRAALSSFGLGGTNTHAIFEQFKRDSDKGKIDGTCIVPISAKNKERLQEYAEDILAYLERRGFENSQLPDFAYTLQVGREAMEHRVVFIADHVNELKQRLTDFINGNTAIEGCFQGSKHNAREVSWLTEDEDSAELIRKWMAKGKVNKLAEMWSKGAHIDWMQLYKGERPNRMSLPTYPFAKERYWPSQDDRKPVAQISGNQTGIGSIHPLLHQNTSDFSEQKFSSVFTGDEFFLRDHVVRGKPVLPGVAYLEMAYAAINQAAGSEIGQDVRIRLNHTVWVQPVVVDRHSAQVDISLFPEEDGKITFDIYSTQEDGDDPVIHSQGSAELASAAETPVADLTEMSRRCGKGKMSPDQFYEEGRSRGMFHGPAFQGIKNVNIGNREVLAQLQLPEIVSGTNEQFVLHPSIMDSALQTATICIMQELTDQKLILPFALEELEVIKGCSSSMWAYARLSDSDHSGGVVQKADIDVIDESGTVCVRIKGFSTRVLEGEVHTSKPSTRHERLMLEPVWEKQNEEREDEDLSYTEHIIVLFETERSVTDSIASHMKDARVITLNEAVGHIAERYQCYMQNIFELLQSKVRKLSAGRIIIQAIVPLEKEKQLFAGVSGLFKTAEIEFSKLTAQVIEIEKPEEMIDLHLKLKDDSRRPFDKQIRYEAGYRFVKGWREMVLPSADTLHMPWRDEGVYLITGGAGSLGLLFAKEIANRTGRSTIVLTGRSVLSEDKENELEALRSIGAEVVYREADVSDQHAVRHLLEEIKERYGTLNGIIHGAGSSKDRFIIHKTNEEFQEVLQPKVSGLLHVDECSKDFPLDFFIFFSSVSGCLGNAGQADYAAANSFMDAFAEYRRSLAASKKRFGSTISFNWPLWEEGGMQVGAEDEKRMLKTTGMVPMPTDSGLKAFYQGIVSDKPQVFVMEGQLQKMKQKLLSAGSKAKRNDQRKADQDQGQTRKLEAALIQMVGAILKVNTDDIDVNTELSEYGFDSVTFTVFTNKINEKFQLELTPTIFFEYGSVQSLAEYVVAAYQGEWNQDATAKGKDERTNLVHSLSSLEASLSNMVSAILKVNSEDIDVNTELSEYGFDSVTFTVFTNKINEEFQLELTPTIFFEYGSLHSLAEYLTVEHGDTLVQEREKPEGQEELQTKSSEAPKITSRRKRRFTQPIIAKAERNKKQAADFEPVAIVGISGRFPGAMDIDEFWKNLEEGKDSITEVPKDRWDWREHYGNPDTDVNKTDIKWGGFIDGVAEFDPLFFGISPREADYVDPQQRLLMTYVWKALEDAGCSPQSLSGTGTGIFIGTGNTGYKDLFHRANLPIEGHAATGHMIPSVGPNRMSYFLNIHGPSEPVETACSSSLVAIHRAVTAMQNGDCEMAIAGGVNTILTEEAHISYSKAGMLSTDGRCKTFSADANGYVRGEGVGMVMLKKLEDAERDGNHIYGVIRGTAENHGGRANTLTSPNPKAQADLLVRAYRQADIDPSTVTYIEAHGTGTELGDPIEINGLKAAFKELSNMRGESQPDVPDHRCGIGSVKSNIGHLELAAGISGLIKVLLQMKHKTLVKSLHCETLNPYLQLTDSPFYIVQEKQEWKSVTDRDGNELPRRAGISSFGIGGVNAHIVIEEYMPKANSEHTATEQPNVIVLSAKNKSRLIDRASQLLEVIRNKKYTDQDLHRIAYTLQVGREEMDERLACVAGTMQELEEKLQAFVDGKEETDEFFRGQSHRNKETQTIFTADEDMALALDAWIRKRKYAKLADLWVKGVSIQWNTLYGETKPRLISLPSYPFAKDHYWVPAKEHSERDKKELVNAIEDRAACFLTKQWSLSPIGSAVPGTRTVAILCCQETADLAAEVSSYFPNHLLIDVSRIENDQSDIDWKEFDGLVDVIGCGWDDEGRLDWIEWVQRLVEFGHKEGLRLLCVTKGLESFQNTSVRMAGASRAGLYRMLQCEYSHLISRHMDAEEVTDHRRLAKLIADEFYSDSYDAEVCYRDGLRYQAFLKAHPETGKATEQSAVFPKDHVLLITGGTRGIGLLCARHFAECYGVKKLVLTGREQLPPREEWARFKTSNTSLAEKIQAVRELEAKGVQVEMLSLTLSDDAQVEQTLQHIKRTLGPIGGVIHCAGLTDMDTLAFIRKTSDDIQRVLEPKVSGLTTLYRHVCNEPLQFFVLFSSVSAIIPELSAGQADYAMANSYMDYFAEAHQKHAPIISVQWPNWKETGMGEVTNQAYRDSGLLSITNSEGLRFLDQIVSKKFGPVVLPAMANQTNWEPELLMKRRKPHEGGLQEAALQSPPARDIEEADEVSKCDGLLSETQSWLIDLFTEELRIDREDFEIDGLFQDYGVDSIILAQVLQRINRKLEAALDPSILYEYPTIQRFADWLIGSYSERLSALFGGRISDASAPLENKIEAEASVPGKDRALTPQIQAPAILSPDSHAEGIAVVGLSCRFPGAETLESYWSLLSEGRSSIGPIPAERWGCKTPYYAGVIDGVSYFDPDFFLLHEEDVRAMDPQALLVLEECLKLLYHAGYTPEEIKGKPVGVYIGGRSQHKPDEDSLDHAKNPIVTVGQNYLAANLSQFFDVRGPSVVVDTACSSALVGMNMAIQALRGGDIQSAIVGGVSLLSSDASHRLFDRRGILSKHSSFHVFDERADGVVLGEGVGMVMLKTVKQALEDGDIIYAVVKAASVNNDGRTAGPATPNLEAQKEVMKDALFKSGKKPEDISYLEANGSGSIVTDLLELKAIQSVYRSGHSSPLSLGSIKPNIGHPLCAEGIASFIKVVLMLKERRFVPFLSGEKEMAHFDQQKANITFSRALEKWTDSQPTAAINCFADGGTNAHVIVEAWEKDEKHAIKRSPISPPQLKKRMLSPGEPKLEAETSKMTAANIWDTYEVEV

SEQ ID NO: 11 - protein, 4538 aa, Bacillus subtilis
MRWRSNVKKITKQLTLSLKNPFIYHHVVYGQNVLPGLAYIDIIYQIFREHGFSCSELQLRNLSIYQPLTAEQDAVIVLNIQCAEKKEGQWQITAKGIEKRDGKEASEEKLYMKADMHADSPAIFEETLDLSQIKASAQNVVQLDDVYEQCRRQELVHSEYMKAKGCIYEEEDGVLLELSLGSEAMLHAEGFMFHPTLIDGSGVGANHLLTSLLKGEQRLYLPLFYESFSASALLQTDCMTRIKRSSVRREKELIYVTLEFFNASGEKVAELKNFTSKLVREAELISGKHQDAQETQMTRADTAERDKPADMVSSPVNSYSEAEQFVSQLIAEKINKPVEQVEKQVGYYQMGLNSSGLLEVVETISDKIGESLSPTLLFEHTTIAELSAFLAEEYAEHFSAAGSLGQNERARVSDSINDHKTVEGSRPAPIEAAGDIAIIGLAGRYPKAANIHEFWNNLKEGKDCVSEIPESRWDWQRLEGITSPSGKDISKWGGFIDDPDCFDPQFFRITPREAETMDPQERLFLETCWETIEDAGYTPKTLAKPKGRNKRQHVGVFAGVMHKDYTLVGAEEASAENVFPLSLNYAQIANRVSYFCNFHGPSMAVDTVCSSSLTAVHLALESIRHGECDVALAGGVNLSLHPNKYMTYGVWDMFSTDGHCRTFGKDGDGYVPAEGIGAVLLKPLRQAEEDGDRIYAVIKGSAVNHVGTVSGISVPSPVSQADLIETCLEKTGIDPRTISYVEAHGTGTSLGDPIEIQGLVKAFRQYTQDRQFCSIGSVKSNIGHAESAAGISGLSKVALQLHHQKLVPSLHSEELNPYVDFEKSPFYVQHETETWKQPVIKENGEDVPYPRRAGISSFGATGSNAHIILEEHIPQAAEQDVSLSSDSDISAVIPLSARNQERLRVYAKRLLDFLHDGIQIRDLAYTLQVGREPMEERVSFLASGIQELSDQLKAFIEGRKAIQHCWKGRVSRGSEPSRPAESVHKLLEQRKLDQIAEQWANGSGVDWKLLYEGSKPKRISLPTYPFERVRYWVPKAEKKTDRSKQERHILHPLLHQNVSDISGVRFRSAFTGREFFLKDHVIKGEHVLPGAALLEMVRAAVERAAADQFPTGFRLRNIVWVRPFAVTEQQKDIDVRLYPEENGEITFEICRDPESAEESPIVYGQGSAVLCEAGENPVINIEELKASYNGRTLSPFDCYEAYTEMGIHYGDSHRAIDSLYAGENGVLVKLTMPPVISDTEDHYILHPSMIDSAFQASIGLRLGGATSLEDRKAMLPFAIQDVRIFKGCEASMWARITYSEGSTAGDRMQKLDIDLCNEEGQVCVRLTSYSARVLETDQEGPSEANDTLLFEHIWEERAAERQELIEYDTYKVVVCDVGEQMESLQNHLDCTVLQHDTETIDERFEGYAIQLFEEIKQLMHSKTGGHTFIQVAVPALDEPQLLSGLTGLLKTAELENPKLTGQLIEIETGMSAGELFEILEENRRYPRDTHIRHWQGKRFVSKWKEVSGEHLSADMPWKDKGVYLITGGAGGLGFIFATEIANQTNDAVVILTGRSPLDERKKKKLKALQKLGIQAIYRQADLADKQTVDALLKETQNVYGDLDGIIHSAGLIKDNFIMKKKKEEVQTVLAPKVAGLIHLDEATKDIPLDFFILFSSGAGAVGSAGQADYAMANAFMNAFSEYRNGQAELHKRYGKTLSVCWPLWKDGGMQIDAETARMLKRETGMVAMETDRGIQALYHGWTSGKPQVLVASGVTDRIRAFLHETGHGKGQSHNIKKSSLNQEAEKADMIGEIDEEILREKAENYFKQVLSSVIKLPAGQIDAEAPLEDYGIDSIMIMHVTGQLEKVFGSLSKTLFFEYQDIRSLTRYFIDSRREKLLDILGFETGKPSVERKSEPEKQEIPVIPRKSGFLPLQDKEQKQVREKETEEIAIIGISGRYPQADNIDELWEKLRDGRDCITEIPADRWDHSLYYDEDKDKPGKTYSKWGGFMKDVDKFDPQFFHISPREAKLMDPQERLFLQCVYETMEDAGYTREHLGRKRDAELGGSVGVYVGVMYEEYQLYGAQEQVRGRSLALTGNPSSIANRVSYYFDFHGPSIALDTMCSSSLTAIHLACQSLQRGECEAAFAGGVNVSIHPNKYLMLGQNKFMSSKGRCESFGQGGDGYVPGEGVGAVLLKPLSKAVEDGDHIYGIIKGTAINHGGKTNGYSVPNPNAQADVIKKAFVEAKVDPRTVSYIEAHGTGTSLGDPIEITGLSKVFTQETDDKQFCAIGSAKSNIGHCESAAGIAGVTKVLLQMKYRQLAPSLHSNVLNPNIDFLNSPFKVQQELEEWKRPIISVNGKDIELPRIAGVSSFGAGGVNAHILIEEYAPEPVEERLPARKQPAVIVLSAKNEERLQKRAERLLHAIREQTYVEADLHRIAYTLQVGREAMKERLAFVAETMQELEEKLYECISGTENREYVYRGQVKSNKEAIAAFAADEDMSKTIEAWLQKGKYAKVLDLWVRGLRIDWSTLYQDQKPRRISLPAYPFARDRYWIDVNAKAEEKRTEEPFAPVQPVIPKPSVDREASGKPANITLQPLMTNQDRLERVPSDTETETITAEALCDELTAGLAEVLYMDQNEIDPDEAFIDIGMDSITGLEWIKAINKQYGTSLNVTKVYDYPTTRDFAVYLAHELSTQAGEKKQTETYTPIRQKTVVPAAKPANISLQPLEHHQPVQEEAEETIQYAAAEISASRQYTVAIETLHENLRESIADVLYMEPYEVDIDEAFIDIGMDSITGLEWIKAVNKQYGTSFTVTRVYDYPTIRDFAEMLKSELGTHLDRKIEHTDSFEAAQQKPAASSHPKPAERPLQPVQHPIKKEHEKKTVPVLQDRPEDAIAIVGMSGRYPGARNVREYWDNLVHARNAIRDIPTSRWDVDKYYDPVLNKKGKVYCKSMGMLDDIEHFDPLFFNIPPSEAELMDPQHRIFLQEGYKAFEDAGYNARTLNEKKCGVYLGIMSNEYGVMLNRQSRANATGNSFAIAAARIPYFLNLKGPAIPIDTACSSSLVGTHLARQALINKEIDMALVGGVSLYLTPESYMSMCEAGMLSPDGQCKAFDNGANGFVPGEGAGALVLKRLKDAEADRDHIYGIIIGSGINQDGKTNGITAPSAKSQMDLERDIYETYGIHPESISYVEMHGTGTKQGDPIELEALSTVFQEKTDKKQFCAIGSVKSNIGHTSAAAGVAGVQKVLLCMNHKTLVPTLNFTTPNEHFEFEHSPLYVNTELKPWETADGKPRRACVSSFGYSGTNAHIVIEEYQPEKRNDRLTKQHRSALFVLSAKKEKQLKAYAEAMKDFVTSNEDIDLEDMAYTLQTGREAMDYRMAFLADSREMLIKALDDYLAEMPNGSIFAAHVKTKKSEIKLFETDHDAKALLQTWIEKKRLEKVAELWVKGLQIDWNKLYGEYTPRRISLPAYPFAEEYYWLPTQEGEPETIATAMPQFELMPKRCFLRKQWQPCPIEPAEMTNQTVAILANEETMALAEELSAYFSTYRIFDSQELDRVSAADYEHVAGAIDLIGCGTSHEHSMGWINWLQKLIEQGRASKHHLTVLGVTKGLEAYANEGVLLSGASRAGLYRMLQSEYSHLTSRHADMECEASHEELARLIAVEYYAKSTESEVCYRNGQRYRAYLTEQPAEAALSHKQVSFSTDKVLLITGGTRGLGLLCARHFVKTYGVKRLVLIGREELPPRDQWNSVKISSLAEKIKAVQELEDMGAQVQVLSLDLTDRVAVEQSLKTIHETMGAIGGVIHCAGMVNKQNPAFIRKSLEEIGQVLEPKVEGLQTLFDLLQDEPLAFFTLFSSVSAAIPALAAGQADYAMANAFMDYFAEAHQDKCPIVSIQWPNWKETGLGEVRSKALEQTGLISLTNDEGLQLLDQILSDRQYAVVLPAVPDTNVWKPDKLMQPSLPVEALSHPETKEQTSTRNLFPETVDWLVTLFSDELKIAAEDFETDEPFQEYGIDSIILAQLVQQMNQQLNGDIDPSILFEYPTIESFAHWLISKYDISAVLQPSVPEKQTPLKPQSAMKQKLVPEQRPQQISHEKTALLAEDIAIIGLSCRFPGAETLEEYWDLIRDGRSAIAPVPPERFGNSSSNYAGLIDEMNRFDHDFFMMSESDVRAMDPQALAVLEESLKLWYHAGYTEKEVKGMRAGVYIGGRSQHKPDPASLSKAKNPIVAGGQNYLAANISQFFDLKGPSIVLDTACSSALVGLNMAIQALRSGDIEAAVVGGVSLLDADAHRMFHERGLLCDKPSFHIFDKRADGVILGEGVGMVLVKTVNQAVEDGDSIYAVIKAAAINNDGRTAGPSSPNLEAQKDVMLSALEKSGKKTEEISYLEANGSGSAVTDLLELKAIQSIYRSESKAPLGLGSVKPNIGHPLCAEGIASLIKVALMLKHRQLVPFLSGNENMPYFDIEKTDLYFSRSQAEWKETTPAAAINCFADGGTNAHLIIEGWRDSAERPIRRKPLPLPELNRQPVLIKPSAQNVQKKVHSDTGASKDMFWKTFK

SEQ ID NO: 12 - protein, 483 aa, Bacillus subtilis
MSSNKLTTSWGAPVGDNQNSMTAGSRGPTLIQDVHLLEKLAHFNRERVPERVVHAKGAGAHGYFEVTNDVTKYTKAAFLSEVGKRTPLFIRFSTVAGELGSADTVRDPRGFAVKFYTEEGNYDIVGNNTPVFFIRDAIKFPDFIHTQKRDPKTHLKNPTAVWDFWSLSPESLHQVTILMSDRGIPATLRHMHGFGSHTFKWTNAEGEGVWIKYHFKTEQGVKNLDVNTAAKIAGENPDYHTEDLFNAIENGDYPAWKLYVQIMPLEDANTYRFDPFDVTKVWSQKDYPLIEVGRMVLDRNPENYFAEVEQATFSPGTLVPGIDVSPDKMLQGRLFAYHDAHRYRVGANHQALPINRARNKVNNYQRDGQMRFDDNGGGSVYYEPNSFGGPKESPEDKQAAYPVQGIADSVSYDHYDHYTQAGDLYRLMSEDERTRLVENIVNAMKPVEKEEIKLRQIEHFYKADPEYGKRVAEGLGLPIKKDS

SEQ ID NO: 13 - DNA, 25 bp, Artificial Sequence
  Primer
gtacatattggcagaaacagctatc

SEQ ID NO: 14 - DNA, 25 bp, Artificial Sequence
  Primer
cctggtgtatgtatcctctctaaac

SEQ ID NO: 15 - DNA, 25 bp, Artificial Sequence
  Probe
tgtcagcgcaagttcagtcgattct

SEQ ID NO: 16 - DNA, 22 bp, Artificial Sequence
  Primer
ccgaagctgttaggctcgtaat

SEQ ID NO: 17 - DNA, 20 bp, Artificial Sequence
  Primer
cgcgcacgcaacaaagtaaa

SEQ ID NO: 18 - DNA, 26 bp, Artificial Sequence
  Probe
cgcatttgcccatcacgctgataatt

SEQ ID NO: 19 - DNA, 23 bp, Artificial Sequence
  Primer
tttgggtctacatggtggataag

SEQ ID NO: 20 - DNA, 24 bp, Artificial Sequence
  Primer
aaccctaatcttactggtcagttc

SEQ ID NO: 21 - DNA, 28 bp, Artificial Sequence
  Probe
tgcaggatcatagctgatctcaaatcgc

SEQ ID NO: 22 - protein, 187 aa, Bacillus subtilis
MLNKIAELINGDGELKNAKIHHSLYAKLEAAATSENVDTDHYINHLLNETIIRSENEQMQEEKKIEIHKNAKPKYKKLDTYLDATLNRNVLIGITNELAGKETGQDIYTLTDYFIDYPACSLWLNLTRHGEQRPIRFNNIKDIEFIELPENRLKVWVYMVDKTWRFEISYDPAIQKITNELTSKIRV

(19) United States
(12) Patent Application Publication
(10) Pub. No.: US 2020/0359652 A1
(43) Pub. Date: Nov. 19, 2020

(54) BACTERIAL STRAIN COMPOSITIONS AND METHODS OF USING SAME

(71) Applicant: AGRI-KING, INC., Fulton, IL (US)

(72) Inventors: Gbenga Ayangbile, Fulton, IL (US); Mary Grzemski, Fulton, IL (US); David Spangler, Fulton, IL (US); Lucas Krueger, Fulton, IL (US)

(21) Appl. No.: 15/929,579

(22) Filed: May 11, 2020

Related U.S. Application Data
(60) Provisional application No. 62/849,276, filed on May 17, 2019.

(51) Int. Cl. A23K 10/18; C12N 1/20; C07K 14/32; C12R 1/125; A23K 10/12; A23K 20/24; A23K 20/147; A23L 13/40; A23L 15/00; A23C 9/152

(52) U.S. Cl. CPC A23K 10/18; C12N 1/20; C07K 14/32; C12R 1/125; A23K 10/12; A23K 20/24; A23V 2002/00; A23L 13/432; A23L 13/43; A23L 15/30; A23C 9/1522; A23C 9/1528; A23K 20/147

(57) ABSTRACT

Compositions having surfactant and carbohydrate-degrading activity are produced from Bacillus subtilis strain 6A-1. The compositions include cultured plaque, exudate and fractions having such activity. Methods of producing the compositions and compositions which have increased surfactant and carbohydrate-degrading activity and increased biomass are provided. Proteins and nucleic acid molecules associated with same and methods of identifying compositions comprising the surfactant and carbohydrate-degrading activity are provided. Feeding the compositions to animals results in increased unsaturated and/or decreased saturated fatty acids in the animals and their food products and can also result in increased absorption and/or retention of dietary calcium by said animals.

### REFERENCE TO RELATED APPLICATION

This application claims priority to previously filed and co-pending provisional application U.S. Ser. No. 62/849,276, filed May 17, 2019, the contents of which are incorporated herein by reference.

### SEQUENCE LISTING

The instant application contains a Sequence Listing which has been submitted in ASCII format via EFS-Web and is hereby incorporated by reference in its entirety. Said ASCII copy, created on May 5, 2020 is named P12858US01_SEQ_LISTING_ST25 and is 117,655 bytes in size.

### BACKGROUND

Bacillus subtilis as source of bioactive exudates Genus Bacillus consists of gram positive aerobic spore-formers found ubiquitously in soil and produces a variety of products that are commercially useful. Bacillus subtilis has long been recognized as a source of enzymes (Priest, F. G. “Extracellular Enzyme Synthesis in the Genus Bacillus.” Bacteriol. Rev. 41(3):711-753 (1977)), vitamins (Sumi, H. U.S. Pat. No. 6,677,143 B2. “Method for Culturing Bacillus subtilis natto to Produce Water-Soluble Vitamin K and Food Product, Beverage or Feed Containing the Cultured Microorganism or the Vitamin K Derivative.” (2004)) and surface active (surfactant) exudates (Geys, R., Soetaert, W., and Van Bogaert, I. “Biotechnological Opportunities in Biosurfactant Production,” Curr. Opin. Biotechnol. 30:66-72 (2014)). Collectively, exudates of B. subtilis have been categorized as exopolymeric substances comprised of polysaccharides, lipopolysaccharides, glycolipids, bioactive proteins, and small peptides or lipopeptides (Marvasi, M., Visscher, P. T., and Martinez, L. C. “Exopolymeric substances (EPS) from Bacillus subtilis: Polymers and genes encoding their synthesis.” FEMS Microbiol. Lett., 313:1-9 (2010)).

### SUMMARY

Here provided are compositions having surfactant activity and carbohydrate degrading activity. The compositions are produced from Bacillus subtilis strain 6A-1 and can include cultured plaque, exudate and fractions produced therefrom. Methods of increasing the total biomass, surfactant activity and carbohydrate degrading activity are provided. In embodiments this may include culturing on solid-phase media, scraping cultured plaque, diluting, centrifuging, filtering, drying the plaque or exudates, extracting with a solvent, or a combination thereof. Proteins in compositions that have surfactant activity are SEQ ID NO: 2 or SEQ ID NO: 3 or those with at least 95% identity thereto. Proteins in compositions that have carbohydrate degrading activity comprise SEQ ID NO: 4 and may also include SEQ ID NO: 8 or sequences with 95% identity thereto. Embodiments provide the compositions comprise nucleic acid molecule SEQ ID NO: 1 or a sequence having 95% identity thereto or a polypeptide comprising SEQ ID NO: 22 or a sequence having at least 95% identity thereto. Feeding the surfactant composition to animals results in increased unsaturated fatty acid and/or decreased saturated fatty acid in the animal or food produced therefrom. Feeding the animal the compositions results in increased calcium absorption and/or retention and reduced calcium in animal waste.

### BRIEF DESCRIPTION OF THE DRAWINGS

FIG. 1 is a gel showing separation of proteins in 2 dimensions, where proteins are separated by isoelectric point (pH units) along the horizontal axis and by size (kDa) along the vertical axis.

### DESCRIPTION

Described here are methods of producing cultured plaque and exudates of bacterial strain Bacillus subtilis subsp. subtilis 6A-1 (6A-1), reference strain having been deposited as ATCC Deposition Number PTA-125135, methods of refining said cultured plaque to enrich for emulsifying bioactivity enacted by one or more surface-active emulsifying agents (surfactant) such as that found with SEQ ID NO: 2 and/or 3, or to enrich for carbohydrate degrading bioactivity, and compositions comprising said cultured plaque or refined plaque or exudates. The plaque is a plurality of strain 6A-1 bacteria of said strain, and in the present methods, is cultured on a medium (such as produced by human or machine) providing nutrients as opposed to being collected in the wild. Further embodiments provide the culture is a solid phase culture as opposed to a liquid phase culture. Embodiments provide for a cultured plaque that has been scraped, that is removed, from the medium. Refined plaque refers to the plaque having been subject to further processing as described here. In an embodiment, for example, this includes any combination of diluting, centrifuging, filtering and/or drying. Any method of drying such as freeze drying, drying under heat, drying under vacuum, spray drying or other various methods may be employed to the plaque or any exudates. Further embodiments provide for solvent extraction of the exudates which may be combined with any of the above processes. An embodiment provides solvent extraction obtains a non-polar composition comprising surfactants. When referring to exudates is meant 6A-1 substances produced by or from the strain or cultured plaque. When referring to extract is mean a subset of said exudates. The exudate may be free of 6A-1 cells in an embodiment. Certain embodiments provide the fractions are surfactant fractions that enact increased absorption and/or retention of dietary calcium by animals. The exudate or extract may be one that comprises a desired component such as one having surfactant activity or calcium absorption and/or retention activity, or a cellulose degrading activity for example. The exudate may be obtained by any convenient method, and those that provide for improved amounts of the desired component are described herein. In additional embodiments the compositions may optionally include the 6A-1 strain, cells or spores. Methods of enriching surfactant activity, carbohydrate degrading bioactivity, amount of biomass including total cultured biomass are further described herein. The surfactant is non-toxic to animals. When a surfactant composition as described herein produced from bacterial strain 6A-1 is fed to an animal, it decreases saturated fatty acids and/or increases unsaturated fatty acid composition of the animal and food products produced from the animal. Further, a composition produced from strain 6A-1 comprising proteins SEQ ID NO: 2 and 3 (CAB1506.1 and CAB15055.2) when fed to an animal will increase calcium absorption and/or retention in the animal. The composition when fed to an animal also results in reduced calcium excreted in animal waste. The compositions produced here in embodiments comprises the nucleic acid sequence of SEQ ID NO: 1 (sequence 6360-1) or a sequence having 95% identity thereto, and/or the amino acid sequence encoded by SEQ ID NO: 1 or a sequence having 95% identity thereto. In one example the amino acid encoded is SEQ ID NO: 22 or a sequence having 95% identity thereto.

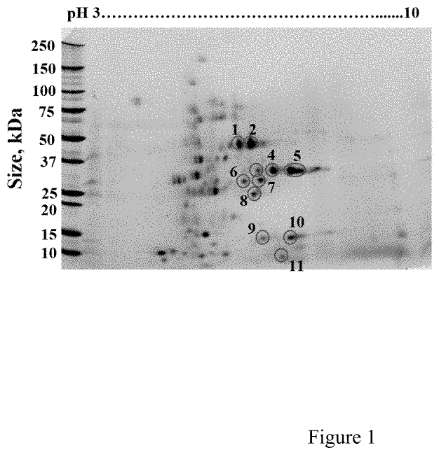

It is understood the composition may also comprise a carrier, excipient and/or diluent appropriate for the process in which it will be used. Examples of such are provided in U.S. Pat. No. 10,138,444, the contents of which are incorporated herein by reference in its entirety. See in particular FIGS. 1-16 of U.S. Pat. No. 10,138,444 all of which are incorporated by reference. Where administered to an animal, it will be non-toxic to the animal. The carrier, excipient and/or diluent is provided to provide improved properties of the composition, such as standardizing, preserving and stabilizing, allowing the bacteria or component to survive the manufacture of animal feed or to survive the digestive system of an animal, lubrication, and improve delivery. In an embodiment the diluent includes a diluent that is not water. There are a myriad of such agents available which may be added. Without intending to be limiting, examples include wetting agents and lubricating agents, preservative agents, lipids, stabilizers, solubilizers and emulsifiers such as examples provided below.

The strain is shown here to produce greater surfactant into cultured plaque than other B. subtilis strains, where surfactant is quantified in cultured plaque by colorimetric assay for methylene blue active substances, where the biological constituents of surfactant are identified as excreted proteins of strain 6A-1, where strain 6A-1 as the strain of origin is identified by DNA in cultured plaque or in refined plaque that comprises a composition having said surfactant bioactivity, and where a composition comprising said cultured plaque or refined plaque with surfactant bioactivity is useful as an animal feed additive for the emulsification of dietary fats or oils. Novel methods for producing cultured plaque of strain 6A-1 are presented, whereby said plaque is produced by culturing spores of strain 6A-1 aerobically on solid phase media and said plaque is harvested by scraping and whereby said solid phase media is comprised of nutritive enrichments to support maximal production of said cultured plaque or maximal concentration of surfactant in said cultured plaque. Similarly, novel methods for producing cultured plaque of strain 6A-1 on solid phase media are presented, whereby said cultured plaque is enriched for carbohydrate degrading bioactivity. Novel methods for producing a composition comprising aggregate extra-cellular constituents of said cultured plaque also are presented, whereby said cultured plaque may be refined to exclude constituents comprising intact cells and spores of strain 6A-1 and where said composition retains surfactant bioactivity, carbohydrate degrading bioactivity, and in embodiments comprises DNA of strain 6A-1. Lastly, a novel method for producing a composition comprising surfactant residues is presented, whereby said surfactant residues are refined from cultured plaque of strain 6A-1 or extra-cellular constituents of said cultured plaque by means of solvent separation. Any of the compositions produced by said methods have utility as an animal feed supplement, where surfactant bioactivity of the respective composition is projected to enact the emulsification of dietary fats and expose fatty acids in aqueous digestive fluids to promote the formation of calcium salts of fatty acids. Upon increased absorption of calcium salts, animals fed any of the compositions comprised of cultured plaque or refined plaque of strain 6A-1 are projected to increasingly retain calcium in bodily tissues and produce animal food products with greater content or proportion of any of the unsaturated fatty acids, especially 18:1, 18:2, or 18:3 fatty acids, or lower content or proportion of any of the saturated fatty acids, especially palmitic acid (16:0).

Specifically, the surface-active lipopeptides are known to emulsify substrates that would otherwise be insoluble (Neu, T. R. “Significance of Bacterial Surface-Active Compounds in Interaction of Bacteria with Interfaces.” Microbiol. Rev. 60(1):151-166 (1996)). Surface-active lipopeptides such as surfactins, iturins and plipostatin-fengycins are small molecules containing 7-10 amino acids as a cyclic peptide that is bound to a fatty acid chain. Surface active lipopeptides therefore can maintain hydrophilic character at the cyclic peptide and hydrophobic character at the fatty acid chain. This property causes the surfactant lipopeptide exudates of B. subtilis to collect at the interfaces between liquids with differing polarity where they are known to reduce surface and interfacial tension (Gundina, E. J., Fernandes, E. C., Rodrigues, A. I., Teixeira, J. A., and Rodriques, L. R. “Biosurfactant Production by Bacillus subtilis Using Corn Steep Liquor as Culture Medium.” Front. Microbiol. 6:1-7 (2015)).

Additionally, biofilm surface layer protein A, or BslA, is a known protein exudate of B. subtilis that confers hydrophobicity to the biofilm of said bacterial species (Kobayashi, K. and Megumi I. “IBslA (YuaB) forms a hydrophobic layer on the surface of Bacillus subtilis biofilms.” Mol. Microbiol. 85(1):51-66 (2012)) and is structurally defined as a hydrophobin (Hobley, L., Ostrowski, A., Rao, F. V., Bromley, K. M., Porter, M. Prescott, A. R., MacPhee, C. E., Van Aalten, D. M. F., and Stanley-Wall, N. R. “BslA is a self-assembling bacterial hydrophobin that coats the Bacillus subtilis biofilm” Proc. Nat. Acad. Sci. 110(33):13600-13605 (2013)). However, unlike the broader class of hydrophobins, protein BslA has been shown to undergo a conformational re-arrangement upon exposure to an aqueous or hydrophobic environnent, respectively, whereby the protein is recognized as a natural surfactant (Morris, R. J., Schor, M. Gillespie, R. M., Ferreira, A. S., Baldauf, L. Earl, C. Ostrowski, A. Hobley, L., Bromley, K. M., Sukhodub, T. and Arnaouteli, S. “Natural variations in the biolilm-associated protein BslA from the genus Bacillus.” Sci. Rep. 7:1-13 (2017)). Surfactants, whether synthetic or biologically natural, have been used in many different applications such as detergents, bioremediation, and pesticides (Geys, R., Soetaert, W., and Van Bogaert, I. “Biotechnological Opportunities in Biosurfactant Production,” Curr. Opin. Biotechnol. 30:66-72 (2014)), and utility of biosurfactants in food processing also has been noted, especially for the stabilization of foamed or aerated emulsions (Tchuenbou-Maggaia, F. L., Norton, I. T., and Cox, P. W. “Hydrophobins stabilized air-filled emulsions for the food industry. Food Hydrocolloid. 23(7):1877-1885 (2009); Green, A. J., Littlejohn, K. A., Hooley, P. and Cox, P. W. “Formation and stability of food foams and aerated emulsions: Hydrophobins as novel functional ingredients. Curr. Opin. Colloid. In. 18(4):292-301 (2013)).

### Surfactants as Animal Feed Additives

Some synthetic surfactants have been researched as feed additives to animals. In nearly all cases, the hypothesized mode of action has been the enactment of emulsion of dietary oils in aqueous digestive fluids, but effects of synthetic surfactant supplementation to animals have not been consistently beneficial. For example, Tween 80, which is a synthetic surfactant, was shown not to influence the digestion or feeding value of added fat when supplemented to Holstein steers (Davila-Ramos, H., Gonzalez-Castellon, A., Barreras-Serrano, A., Estrada-Angulo, A., Lopez-Soto, M. A., Macias-Zamora, J. V., A Plascencia, A. Vega, S. H., and Zinn, R. A. “Influence of Method of Surfactant Supplementation on Characteristics of Digestion and Feeding Value of Fat in Holstein Steer Fed a High-Energy Finishing Diet.” J. Appl. Anim. Res. 39(3):192-195 (2011)). Similarly, supplementation of Tween 80 was shown to decrease feed intake and increase the number of days for growing lambs to reach slaughter weight (McAllister, T. A., Stanford, K., Bae, H. D., Treacher, R. J., Hristov, A. N., Baah, J., Shelford, J. A., and Cheng, K-J. “Effect of a Surfactant and Exogenous Enzymes on Digestibility of Feed and on Growth Performance and Carcass Traits of Lambs,” Can. J. Anim. Sci. 80(1):35-44 (2000)). However, the supplementation of alkyl polyglycoside, which is also a synthetic surfactant, was shown to increase production of fluid milk and milk solids when supplemented to dairy cows (Zhang, X., Jiang, C., Gao, Q., Wu, D., Tang, S., Tan, Z, and Han, X. “Effects of Dietary Alkyl Polyglycoside Supplementation on Lactation Performance, Blood Parameters, and Nutrient Digestibility in Dairy Cows”, Animals 9(8):549 (2019)). Similarly, supplementation of a non-ionic surfactant to dairy cows has been shown to increase the bioactivity of digestive enzymes such as cellulase, xylanase, amylase, and protease, which is projected to be favorable for productive performance (Lee, S. S., Kim, H. S., Moon, Y. H., Choi, N. J and Fla, J. K. “The effects of a non-ionic surfactant on the fermentation characteristics, microbial growth, enzyme activity and digestibility in the rumen of cows”. Anim. Feed Sci. Tech 115:37-50 (2004)).

Examples in the body of scientific literature of the effects of dietary surfactant on the composition of fatty acids in digestive fluids, where supplemental surfactant was hypothesized to enact emulsification of dietary oils, are limited to a single study to the authors' knowledge. In a single example, the composition of fatty acids in rumen fluid was altered by supplementing alkyl polyglycoside to goats, but the proportion of mono-unsaturated fatty acids was decreased in rumen fluid, and the fatty acid composition of animal tissue in response to supplementation specifically was not evaluated (Zeng, B., Tan, Z., Zeng, J., Tang, S., Tan, C., Zou, C., Han, X. and Zhong, R. “Effects of dietary non-ionic surfactant and forage to concentrate ratio on bacterial population and fatty acid composition of rumen bacteria and plasma of goats”. Anim. Feed Sci. Tech. 173:167-176 (2012)). Surfactants produced by B. subtilis have been researched more heavily, but not for emulsifying bioactivity. The lipopeptides comprising surfactin of B. subtilis and other similar excreted small molecules have been shown to have broadly antimicrobial properties (Cameotra, S. S., and Makker, R. S. “Recent Applications of Biosurfactant as Biological and Immunological Molecules,” Curr. Opin. Microbiol. 7:262-266 (2004)) and thus have been researched extensively for utility in conferring protection against infectious disease in food animals (Cheng, Y. H., Zhang, N., Han, J. C., Chang, C. W., Hsiao, F. S. H. and Yu, Y. I. “Optimization of surfactin production from Bacillus subtilis in fermentation and its effects on Clostridium perfringens-induced necrotic enteritis and growth performance in broilers.” J. Anim. Physiol. Anim. Nutr. 102(5):1232-1244 (2018)).

Importantly, B. subtilis is generally recognized as safe (GRAS) in the United States for use in animal feed (Official Publication of the Association of American Feed Control Officials. AAFCO. Ingredient. Number 36.11 p 389 (2020)), but strains of B. subtilis known to make surfactin-like lipopeptides that confer cytotoxicity are prohibited from supplementation to food animals in the European Union (EFSA Panel on Additives and Products or Substances used in Animal Feed (FEEDAP). “Guidance on the assessment of the toxigenic potential of Bacillus species used in animal nutrition.” EFSA Journal 12(5):3665 (2014)). Therefore, exudates of B. subtilis that comprise surfactant bioactivity have not been researched specifically for the utility of enacting emulsification of dietary oils in digestive fluids. Furthermore, exudates of B. subtilis that confer surfactant activity, other than small lipopeptides, have not been researched for utility as animal feed additives, but are of potential value where small lipopeptides that confer similar activity cannot be utilized as additives. Specifically, biofilm surface layer protein A, or BsA, referenced herein by GenBank accession number CAB15086.1, has not been researched for any utility as an animal feed additive, especially for the purpose of enacting a biochemical change in the composition of a food animal or a food product from a food animal. The closest projected utility for biofilm surface layer protein A or hydrophobins of similar molecular character, as has been presented previously herein, is the stabilization of aerated or foamed emulsions during food processing, and this utility is not synonymous with the emulsification of dietary oils in digestive fluids.

Similarly, feeding a surfactant or emulsifier to food animals has been hypothesized to increase the emulsification of dietary fats in digestive fluids, and subsequently, the digestibility of dietary fats, but the implications of the enactment have not been determined. Dietary oils comprised of triglycerides are commonly known to be emulsified in the intestine of ruminant and non-ruminant animals by endogenous bile salts and further digested into constituent free fatty acids and monoglycerides by lipase enzymes of salivary and pancreatic origin. In ruminant species, this direct route of digestion is confounded by microbial hydrogenation of unsaturated fatty acids to saturated fatty acids in the foregut (Polan, C. E., McNeill, J. J. and Tove, S. B. “Biohydrogenation of unsaturated fatty acids by rumen bacteria”. J Bacteriol. 88(4):1056-1064 (1964)).

### Outcomes of Exogenous Surfactant Supplementation to Food-Producing Animals are not Known.

Further increasing emulsification is thought to increase the efficiency of endogenous lipase enzymes (Rovers, M. “Improving fat digestibility with emulsifiers” AllAbout Feed, October 2013). However, calcium in digestive fluids readily forms soap complexes with free fatty acids, where the solubility and absorbability is heavily dependent on the fatty acid constituent of the soap complex. Calcium soaps of saturated fatty acids with chain lengths of 12, 14, 16, or 18 carbons are less than 10 percent absorbed in the intestine, and the calcium soap of stearic acid (18:0) is approximately 1 percent absorbed. In contrast, the calcium soap of oleic acid (18:1) is approximately 10 percent absorbed and the calcium salt of linoleic acid (18:2) is more than 20 percent absorbed (Gacs, G. and Barltrop, D. “Significance of Ca-soap formation for calcium absorption in the rat.” Gut 18:64-68 (1977)).

To the authors' knowledge, the effects of supplementally enacting increased emulsification of dietary oils in digestive fluids have not been demonstrated in the body of scientific literature, especially with respect to calcium homeostasis and absorption of specific dietary fatty acids. Advancements toward understanding these effects have included a demonstration that increasing the concentration of calcium in an oil-in-water emulsion increases the digestion of triglycerides in said emulsion (Hu, M., Li, Y. Decker, E. A., and McClements, D. J. “Role of calcium and calcium-binding agents on the lipase digestibility of emulsified lipids using an in vitro digestion model. Food Hydrocolloid, 24(8):719-725 (2010)). However, an example in literature where supplementation of calcium and lecithin as an emulsifier were supplemented to pigs identified no response in productive performance, but specifically did not measure absorption of calcium from digestive fluids, retention of calcium in tissues, or the composition of fatty acids in animal tissues as a result of lecithin or calcium supplementation (Mitchaothai, J., Yuangklang, C., Vasupen, K., Wongsuthavas, S. and Beynen, A. C. “Effect of dietary calcium and lecithin on growth performance and small intestinal morphology of young wild pigs.” Livest. Sci. 134:106-108 (2010)).

Despite the lack of knowledge regarding the effects of emulsifiers or surfactants fed directly to animals, emulsifiers are known to be useful in vitro for the manufacture of calcium soaps with subsequent utility as an animal feed additive (Perez, E. P., Festo, A. G., Co, K. G., and Norel, S. A. U.S. patent Ser. No. 12/085,841. “Method for Producing Calcium Soaps for Animal Feed.” (2009)). The in vitro manufacture of calcium soaps of fatty acids is readily differentiated from the in vivo complexation of dietary calcium with fatty acids in digestive fluids. In the state of the art, pre-formed calcium soaps are commonly supplemented to animals as a source of supplemental digestible fatty acids, where soaps of unsaturated fatty acids are increasingly resistant to saturation in the foregut of ruminant animals (Wu, Z., Ohajuruka, O. A., and Palmquist, D. L. “Ruminal synthesis, biohydrogenation, and digestibility of fatty acids by dairy cows. J. Dairy Sci. 74:3025-3034 (1991)). Where the method of directly adding a surfactant or emulsifier to animal feed is concerned, said additive is hypothesized to enact emulsification of dietary oils that are intrinsic to other ingredients or exogenously supplemented, where the metabolic fates of calcium and fatty acids that are intrinsic to feed ingredients or exogenously supplemented are unknown in the state of the art. Specifically, to the authors' knowledge, there are no data in the body of scientific literature to identify that feeding an emulsifier to a food-producing animal promotes the formation of calcium soaps of fatty acids in digestive fluids. Subsequently, to the authors' knowledge, there are no data in the body of scientific literature to confirm that the outcome of said mode of action is increased absorption of calcium, retention of calcium or deposition of a specific fatty acid in food animals or food product of food animals. Furthermore, a utility for a surfactant produced by B. subtilis, especially protein BslA, with respect to increasing the absorption of specific fatty acids or calcium, has not previously been demonstrated.

Methods of Manufacturing B. subtilis and Exudates Thereof in the State of the Art

Culturing of Bacillus species is carried out in a laboratory setting, or nonproduction scale, in one of two ways: either using a liquid broth culture or utilizing agar plates. In liquid broth culture, flasks containing sterilized media are inoculated aseptically with Bacillus and incubated at a specific temperature with shaking to allow aeration of the liquid media to occur. Alternatively, agar plates are prepared by adding agar to a liquid nutrient media before sterilization and then pouring media aseptically into sterilized petri plates while the media is warm. The agar solidifies upon cooling to room temperature and then the surface of the media is inoculated with the bacteria. Petri plates are routinely incubated in an inverted position. The method of analytical scale that is utilized, comprising liquid phase or solid phase culture, depends on the desired outcome, which is usually a large volume, or a diffuse culture, or a dense colony.

For commercial production of Bacillus strains, liquid broth cultures are routinely utilized (Korsten, L., and Cook, N. “Optimizing culturing conditions for Bacillus subtilis.” South African Avocado Growers' Association Yearbook, 19:54-58 (1996)). Bacillus species are cultured in large containers called bioreactors that enable scaled volumes of liquid broth to be inoculated and incubated at a regulated temperature and degree of aeration. These cultures are propagated continuously by the addition of fresh, sterilized media and the frequent or discrete removal of cultured product. Said cultures can be established in liquid phase media incorporating agricultural waste or byproducts, as said ingredients provide an inexpensive source of nutrients for bacterial growth (Gundina, E. J., Fernandes, E. C., Rodrigues, A. I., Teixeira, J. A., and Rodriques, L. R. “Biosurfactant Production by Bacillus subtilis Using Corn Steep Liquor as Culture Medium.” Front. Microbiol. 6:1-7 (2015)). By way of example in the above reference, Bacillus subtilis was cultured at analytical scale in liquid-phase media comprised of 5, 10 or 15 percent of corn steep liquor, which is an agricultural byproduct, in 200 ml aliquots in 500 ml flasks and incubated for 24 hours at 37 degrees Celsius.

An alternative method of commercial manufacture is commonly referred to as Koji fermentation, whereby the fermentation method is comprised of culturing Bacillus species or other microorganisms on moistened grain or beans (Yadav, M. M. “Alkaline Protease Production by Isolated Bacillus sp in Submerged and Solid State Fermentation.” J. Bio. Innov. 2(4):161-167 (2013)). Importantly, although referred to as solid-state fermentation, the Koji method is readily differentiated from culturing plaque on a flat culture surface formed, for example, by the addition of agar to a liquid medium, as has been described as an analytical method. As a point of differentiation, culture media of the Koji method is formed as a heterogenous mixture of moistened ingredients, as in semi-solid wheat media presented in U.S. Pat. No. 10,138,444 such that the cultured strain is produced within the Koji media, rather than atop a flat culture surface. A novel method for commercial scale production of B. subtilis strain 6A-1 on a flat culture surface is elaborated further herein.

As previously mentioned, Bacillus species produce desirable exudate products. Extracting said exudates from liquid broth cultures is performed by manipulating a unique feature of the desired exudate product. For example, vitamin K has been extracted from Bacillus subtilis strain natto by fractionating on the basis of water soluble and heat stable properties of the metabolite (Sumi, H. U.S. Pat. No. 6,677,143 B2. “Method for Culturing Bacillus subtilis natto to Produce Water-Soluble Vitamin K and Food Product, Beverage or Feed Containing the Cultured Microorganism or the Vitamin K Derivative.” (2004)). In the method of the example, the bacterial culture comprised of both bacterial cells and exudate products was dried by vacuum, heat, air or freeze drying and then was rehydrated or washed with water. Centrifugation and filtration of the resulting homogenate removed said bacterial cells from solution and an exudate comprising water soluble Vitamin K remained. Elsewhere, gamma-polyglutamic acid was purified from B. subtilis fermentation product by utilizing thermal deactivation of cells followed by filtration and ethanol precipitation (Ho, G. H., Ho, T. I., Hsieh, K. H., Su, Y. C., Lin, P. Y., Yang, J., Yang, K, H., and Yang, S. C. “Gamma-Polyglutamic Acid Produced by Bacillus subtilis (natto): Structural Characteristics, Chemical Properties and Biological Functionalities.” J. Chin. Chem. Soc. 53:1363-1384 (2006)).

Separately, surfactant BL86, also called lichenysin, which is a small lipopeptide that is structurally similar to surfactin (Anuradha, S. N. “Structural and Molecular Characteristics of Lichenysin and its Relationship to Surface Activity” In: Sen, R. (ed) Biosurfactants Advances in Experimental Medicine and Biology. Vol. 672 Springer, New York, N.Y. (2010)) was isolated from Bacillus licheniformis (Horowitz, S., Gilbert, J. N., and Griffin, W. M. “Isolation and Characterization of a Surfactant Produced by Bacillus lichenformis 86.” J. Ind. Microbiol. 6:243-248 (1990)). The poor solubility of the surfactant in acid (pH 2.0) allowed for the desired residue to be obtained by acid precipitation, followed by centrifugation, lyophilization, and solvent extraction.

Notably, the state of the art is absent a method by which Bacillus is cultured at production scale on a flat surface and is separated from culture media without the use of a diluent or a centrifugation method. Furthermore, the state of the art is absent a method by which exudates of cultured Bacillus are retained as a composition without the use of a dehydration step or application of an excipient or carrier.

### Detection of Surfactants in Complex Compositions

A quantitative assay is known for the measurement of surfactants in drinking water. The utility of said method has generally been applied for detection of chemically synthesized surfactants, such as in detergents, in drinking water. The American Society of Testing Measurements has a standard technique for detecting anionic surfactants (ASTM D2330-20, Standard Test Method for Methylene Blue Active Substances, ASTM International, West Conshohocken, Pa., 2020, astm.org). Said technique is a colorimetric assay using ionic pairing of an anionic surfactant with methylene blue reagent (Jurado, E., Fernandez-Serrano, M., Nunez-Olea, J., Luzon, G., and Lechuga, M. “Simplified Spectrophotometric Methods using Methylene Blue for Determining Anionic Surfactants: Applications to the Study of Primary Biodegradation in Aerobic Screening Tests.” Chemosphere 65:278-285 (2006)). As increasing amounts of anionic surfactant are present in solution, increasing amounts of methylene blue reagent bind in solution. Adaptation of this assay by our laboratory for detection of methylene blue active substances in bacterial plaque is elaborated further herein.

### Detection and Analysis of Nucleotide Sequences

As used herein, the terms nucleic acid or polynucleotide refer to deoxyribonucleotides or ribonucleotides and polymers thereof in either single- or double-stranded form. As such, the terms include RNA and DNA, which can be a gene or a portion thereof, a cDNA, a synthetic polydeoxyribonucleic acid sequence, or the like, and can be single-stranded or double-stranded, as well as a DNA/RNA hybrid. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, and mRNA encoded by a gene.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” referred to herein as a “variant” where the alteration results in the substitution of an amino acid with a chemically similar amino acid. Conservative substitution tables providing functionally similar amino acids are well known in the art. See, for example, Davis et al., “Basic Methods in Molecular Biology” Appleton and Lange, Norwalk, Conn. (1994). The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., see, e.g., Creighton, Proteins: Structures and Molecular Properties (WH Freeman and Co.; 2nd edition (December 1993)).

Primers and probes may be used to identify material having the sequence of interest. Primers and probes can be developed which specifically recognize this (these) sequence(s) in the nucleic acid (DNA or RNA) of a sample by way of a molecular biological technique on the basis of sequence complementarity. For instance, a polymerase chain reaction (PCR) method can be developed to identify the presence of the sequence in biological samples (such as samples of bacteria, spores, plaques or products comprising same). Such a PCR is based on two specific “primers”, one recognizing a sequence on the sense (or forward, or coding) strand of double stranded DNA, and the other recognizing a sequence on antisense (or reverse, or non-coding) strand of double stranded DNA. In the case of RNA analysis, said RNA is commonly converted to cyclic DNA (cDNA) by means of reverse transcription, whereupon cDNA is analyzed by quantitative PCR methods. The primers preferably have a sequence of typically between 15 and 35 nucleotides which under optimized PCR conditions “specifically recognize” a sequence within SEQ ID NO: 1, so that a specific fragment (“integration fragment” or discriminating amplicon) is amplified from a nucleic acid sample. Similarly, a fluorescent probe can be hybridized to amplified DNA as a means of specific detection in the PCR method. This means that only the region of SEQ ID NO: 1 which identifies its presence, and no other sequence in the bacteria, is amplified under optimized PCR conditions. PCR primers suitable include oligonucleotides ranging in length from 17 nt to about 30 nt, comprising a nucleotide sequence of at least 17 consecutive nucleotides, preferably 20 consecutive nucleotides, selected from the DNA in of SEQ ID NO: 1. Methods for designing PCR primers and PCR cloning are generally known in the art and are disclosed (Sambrook, J., Fritsch, E. F. and Maniatis, T. (2001) Molecular Cloning: A Laboratory Manual, 3rd Edition. Cold Spring Harbor Laboratory Press, Plainview, N. Y; Innis, M., Gelfand, D. and Sninsky, J. (1995) PCR Strategies. Academic Press, New York; Innis, M., Gelfand, D. and Sninsky, J. (1999) PCR Applications: Protocols for Functional Genomics, Academic Press, New York). Known methods of PCR include, but are not limited to, methods using paired primers, nested primers, degenerate primers, gene-specific primers, vector-specific primers, partially-mismatched primers, and the like. In addition, genes can readily be synthesized by conventional automated techniques.

When referring to hybridization techniques, all or part of a known nucleotide sequence can be used as a probe that selectively hybridizes to other complementary nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides that are complementary to the desired sequence to be detected. Methods for preparation of probes for hybridization and for construction of cDNA and genomic libraries are generally known in the art and are disclosed (Sambrook et al., 2001).

For example, the sequence disclosed herein, or one or more portions thereof, may be used as a probe capable of specifically hybridizing to complementary sequences. To achieve specific hybridization under a variety of conditions, such probes include sequences that are unique among the sequences to be screened and are preferably at least about 10 nucleotides in length, and most preferably at least about 20 nucleotides in length. Such sequences may alternatively be used as PCR primers to amplify complementary sequences from foreign DNA by PCR. Hybridization techniques include hybridization screening of DNA libraries plated as either plaques or colonies (Sambrook et al., 2001).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 50° C. Exemplary high stringency conditions include hybridization in 50% formamide, 1 M NaCl, 0.1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC) −0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≥90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (2001) Molecular Cloning: A Laboratory Manual (3^{rd} ed., Cold Spring Harbor Laboratory Press, Plainview, N.Y.) and Haymes et al. (1985) In: Nucleic Acid Hybridization, a Practical Approach, IRL Press, Washington, D.C.

In general, sequences that correspond to the nucleotide sequences described and hybridize to the nucleotide sequence disclosed herein will be at least 50% homologous, 70% homologous, and even 85%, 86%, 87%, 88%, 89%, 90%, 91%, 92%, 93%, 94%, 95%, 96%, 97%, 98%, 99% homologous or more with the disclosed sequence. That is, the sequence similarity between probe and target may range, sharing at least about 50%, about 70%, and even about 85% or more sequence similarity.

The following terms are used to describe the sequence relationships between two or more nucleic acids or polynucleotides: (a) “reference sequence”, (b) “comparison window”, (c) “sequence identity” and (d) “percentage of sequence identity.”
(a) As used herein, “reference sequence” is a defined sequence used as a basis for sequence comparison. A reference sequence may be a subset or the entirety of a specified sequence; for example, as a segment of a full-length promoter sequence, or the complete promoter sequence.
(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to accurately reflect the similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Optimal alignment of sequences for comparison can use any means to analyze sequence identity (homology) known in the art, e.g., by the progressive alignment method of termed “PILEUP” (Morrison, (1997)Mol. Biol. Evol. 14:428-441, as an example of the use of PILEUP); by the local homology algorithm of Smith and Waterman (Adv. Appl. Math. 2: 482 (1981)); by the homology alignment algorithm of Needleman and Wunsch (J. Mol. Biol. 48:443-453 (1970)); by the search for similarity method of Pearson (Proc. Nat. Acad. Sci. USA 85: 2444 (1988)); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins(1988), Gene 73: 237-244; Corpet (1988), Nucleic Acids Res. 16:10881-10890; Huang, Computer Applications in the Biosciences 8:155-165 (1992); and Pearson (1994), Methods in Mol. Biol. 24:307-331); Pfam (Sonnhammer (1998), Nucleic Acids Res. 26:322-325); TreeAlign (Hein (1994), Methods Mol. Biol. 25:349-364); MEG-ALIGN, and SAM sequence alignment computer programs; or, by manual visual inspection.

Another example of algorithm that is suitable for determining sequence similarity is the BLAST algorithm, which is described in Altschul et al, (1990) J. Mol. Biol. 215: 403-410. The BLAST programs (Basic Local Alignment Search Tool) of Altschul, S. F., et al., searches under default parameters for identity to sequences contained in the BLAST “GENEMBL” database. A sequence can be analyzed for identity to all publicly available DNA sequences contained in the GENEMBL database using the BLASTN algorithm under the default parameters.

Software for performing BLAST analyses is publicly available through the National Center for Biotechnology Information, www.ncbi.nlm.nih.gov/; see also Zhang (1997), Genome Res. 7:649-656 for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al (1990), J. Mol. Biol. 215: 403-410). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff (1992), Proc. Natl. Acad. Sci. USA 89:10915-10919) alignments (B) of 50, expectation (E) of 10, M=5, N=−4, and a comparison of both strands. The term BLAST refers to the BLAST algorithm which performs a statistical analysis of the similarity between two sequences; see, e.g., Karlin (1993), Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In an embodiment, GAP (Global Alignment Program) can be used. GAP uses the algorithm of Needleman and Wunsch (J. Mol. Biol. 48:443-453, 1970) to find the alignment of two complete sequences that maximizes the number of matches and minimizes the number of gaps. Default gap creation penalty values and gap extension penalty values in the commonly used Version 10 of the Wisconsin Package® (Accelrys, Inc., San Diego, Calif.) for protein sequences are 8 and 2, respectively. For nucleotide sequences the default gap creation penalty is 50 while the default gap extension penalty is 3. Percent Similarity is the percent of the symbols that are similar. Symbols that are across from gaps are ignored. A similarity is scored when the scoring matrix value for a pair of symbols is greater than or equal to 0.50, the similarity threshold. A general purpose scoring system is the BLOSUM62 matrix (Henikoff and Henikoff (1993), Proteins 17: 49-61), which is currently the default choice for BLAST programs. BLOSUM62 uses a combination of three matrices to cover all contingencies. Altschul, J. Mol. Biol. 36: 290-300 (1993), herein incorporated by reference in its entirety and is the scoring matrix used in Version 10 of the Wisconsin Package® (Accelrys, Inc., San Diego, Calif.) (see Henikoff and Henikoff (1989) Proc. Natl. Acad. Sci. USA 89:10915).

(c) As used herein, “sequence identity” or “identity” in the context of two nucleic acid sequences makes reference to the residues in the two sequences that are the same when aligned for maximum correspondence over a specified comparison window.
(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Identity to the sequence described would mean a polynucleotide or amino acid sequence having at least 65% sequence identity, more preferably at least 70% sequence identity, more preferably at least 75% sequence identity, more preferably at least 80% identity, more preferably at least 85% 86%, 87%, 88%, 89%, 90%, 91%, 92%, 93%, 94%, 95%, 96%, 97%, 98%, 99% sequence identity.

### Novel Methods and Compositions Therefrom

Disclosed here are methods of producing cultured plaque of bacterial strain Bacillus 5 subtilis subsp. subtilis 6A-1 (hereby referred to as “strain 6A-1”), methods of refining said cultured plaque, compositions produced therefrom, and methods of applying any of said compositions as an animal feed additive, reference strain having been deposited as ATCC Deposition Number PTA-125135. Strain 6A-1 and its production are described in detail at U.S. Pat. No. 10,138,444, the contents of which are incorporated herein by reference in its entirety.

### The Method of Culturing on Solid Phase Media

The methods of the invention are comprised of culturing strain 6A-1 in aerobic or microaerophilic atmospheric conditions on solid media, where the chemical composition of solid media is optimized for the production of maximal plaque mass, or maximal concentration of emulsifying bioactivity conferred by one or more surface active agents (surfactant), or maximal concentration of carbohydrate-degrading bioactivity. Solid phase culture is routinely used in laboratories by adding a solidifying agent such as granulated agar to a liquid broth that is nutritively supportive, and in some cases selective, for the growth of a desired micro-organism. Agar is routinely solubilized in media at temperatures achieved during the autoclaving process, which is used to sterilize media, and remains solubilized in media in liquid form as the temperature cools to approximately 50 degrees C. Agar acts as a solidifying agent upon cooling the media to room temperature, after which solid media remains in the solid phase at standard incubation temperatures between 30 degrees C. and 40 degrees C. Solid phase cultures are commonly established in Petri dishes at analytical scale, where a Petri dish of 6 cm or 10 cm diameter provides surface area of approximately 28.3 cm² or 78.5 cm², respectively. Analytical solid phase cultures are most commonly utilized for the isolation of single bacterial colonies or the enumeration of colony forming units, where culture of the micro-organism to confluency on the media surface is of little value.

An embodiment provides that the culture is on a media having a generally flat surface. A surface that allows for scraping of the plaque from the media will suffice. In an embodiment the bacteria is cultured on a surface that is not concave or convex such that scraping the bacteria from the media is inhibited. The media can be placed on a flat surface, or a flat surface may be formed by placing the media in a container such that a generally flat surface on the side on which the bacteria is cultured forms. It is useful, as discussed further herein, to maintain atmospheric humidity. In one example, a lid on the container can be used to retain humidity. In the method of the invention, solid phase cultures are established in rectangular metal pans of approximate dimensions of 41.9 cm by 30.5 cm to provide approximate surface area of 1,277 cm² each. The dimensions and shape of pans are presented by way of example without limitation. By way of example without limitation, pans are fitted with lids that fully cover the surface of the pan. As the desired production scale is increased, pans and lids can be manufactured to appropriate dimensions.

### Methods for Sterilizing a Pan and Lid Assembly

Pans and lids are sterilized prior to culturing procedures. Without limitation, all of the examples of the method of the invention cited herein have used autoclaving to achieve sterilization, where a pan and lid assembly is autoclaved as a unit and then is allowed to cool as a closed unit in open space, such as on a laboratory bench. Methods such as exposure to flame, chemical treatment, and ultraviolent light exposure also are projected as methods for achieving sterilization of culture pans and lids.

### Description of Generic Media Preparation Methods

In the method of the invention, solid phase culture media is prepared according to procedures that are applied routinely at an analytical scale, whereas the formulation of different culture media results in surprising outcomes of total cultured plaque mass or concentration of carbohydrate degrading bioactivity or surfactant bioactivity within said cultured plaque. Generally, culture media is first prepared as a liquid slurry with granulated agar and then is sterilized by autoclaving. Upon autoclaving, media is tempered to a suitable handling temperature of approximately 50 degrees C., and then is poured as a liquid phase into pre-sterilized pans using aseptic technique. The pan and lid assembly is kept together at all times except for when media is being poured. Media is allowed to cool and harden in the pan and lid assembly. The volume of liquid phase media poured into a single pan is approximately 600 mL, which provides a depth of approximately 0.47 cm for the solid phase media in the pan and lid assembly.

### The Method of Surface Inoculation for Solid Phase Culture

Upon cooling to room temperature and setting of media to the solid phase, lids are lifted from the pan and lid assembly temporarily and media is surface-inoculated with approximately 3.0 mL of a solution comprised of 0.9 percent phosphate buffered saline with approximately 7.41×10⁸ spores per mL for a total application of approximately 2.22×10⁹ spores per pan. The inoculum is applied evenly to the face of the solid phase media by using a pre-sterilized, hard, plastic spatula. The pan and lid assembly is then closed, inverted, and laid horizontally in an incubator so that lids are positioned on the bottom of the assembly, as is standard practice with solid phase culture by Petri dish methods. The pan and lid assembly can be sealed with tape, parafilm, or a similar utility to seal the atmospheric conditions in the interior of the assembly.

### Variables Expected not to Affect Culture Outcomes

In the method of the invention comprising solid phase culture of 6A-1 on solid media, the practice of commercial scale production is projected as a novel method, but some processes are not expected to affect the outcome of the culture process. As discussed above, dimensions, form, and material of the media and any containers such as pans and lids are expected not to affect the outcome of culture provided said materials can be sterilized. However, increasing the surface area of the solid phase culture medium is projected to increase total plaque output. Similarly, the method of sterilization is not expected to affect the outcome of culture provided that sterilization is achieved. Where chemical sterilization is utilized, leaching of sterilization chemicals into culture media is projected to impair culture of strain 6A-1. Agar as a solidifying agent in culture media is considered not to be degradable by strain 6A-1, so the inclusion rate of agar in excess of the concentration required to set the media to solid phase, which is elaborated further herein, is expected not to affect the composition comprising a cultured plaque of strain 6A-1. Similarly, neither the conditioning temperature of media after autoclaving, the method of pouring media into pans, nor the method of setting the media to solid phase by cooling media in pans is projected to affect the composition comprising cultured plaque of strain 6A-1.

### Variables

Variables related to the process of culturing on solid phase media, other than the chemical composition of said media that can be optionally modified as desired, that are projected to affect the composition comprising cultured plaque of strain 6A-1 include the inoculation rate of spores, the evenness of inoculum application, the sterility of media, incubation temperature, and atmospheric oxygenation and humidity during incubation. The inoculation rate of spores is projected to affect the total mass of cultured 6A-1 plaque produced on a generally flat surface, here contained in a pan, where growth as a confluent bacterial lawn is a prerequisite for maximal plaque production. Inoculation with 2.22×10⁹ spores, as with numerous pan culture procedures described herein, has been observed to support culture of a confluent bacterial lawn in all cases. Greater application of spores can be utilized but is not expected to increase production of cultured plaque mass, whereas lower application of spores is expected to decrease production of cultured plaque mass, especially at application rates where growth to confluency is not achieved. The evenness of inoculum application to solid media also can improve production of cultured plaque mass, especially where uneven or incomplete application of said inoculum to media decreases the production of total cultured plaque. Incubation temperature has been disclosed in detail in U.S. Pat. No. 10,138,444, where incubation temperature between 30 and 35 degrees C. supports maximal vegetation of strain 6A-1.

Achieving sterility in solid-phase media culture is a prerequisite for producing cultured plaque of strain 6A-1 that has utility as an animal feed additive, but methods for achieving sterility of media are dependent on the type of media. Tryptic soy broth or TSB is a standard bacterial growth medium used for the culture of Bacillus subtilis and can be obtained commercially (BD™ Tryptic Soy Broth, Becton Dickinson, Franklin Lakes, N.J. 07417) in powder form. Per manufacturer's specifications, TSB is formulated at 30 g per L in deionized or distilled water. Tryptic soy broth can be set as solid phase media by the addition of agar to form tryptic soy agar or TSA. By way of example without limitation, granulated agar (Difco™ granulated agar, Becton Dickinson, Franklin Lakes, N.J. 07417) is included in TSB at 25 g per L before sterilization by autoclaving. Example 1 demonstrates that standard autoclave time for liquids of 15 min at standard temperature and pressure of 121 degrees C. and 15 psi, respectively, is sufficient for achieving media sterility that remains sterile after being poured and set as solid-phase media and held at incubation temperature of 35 degrees C. for up to 48 hours. Example 1 also demonstrates that standard autoclave time of 15 min is insufficient for achieving sterility of an alternative media, which is wheat bran agar with up to 9 percent inclusion of wheat bran media powder and is elaborated further herein.

Wheat bran agar (WBA) is presented here as a novel culture medium that contains 30 degradable polysaccharides for the culture of strain 6A-1 or other strains of Bacillus subtilis. The precise composition of the wheat bran agar may vary and the following is provided by way of example without intending to be limiting. Wheat bran agar media, 9 percent, is comprised of 90 g wheat bran media powder and 20 g granulated agar suspended or dissolved per L in deionized or distilled water, where wheat bran media powder is comprised, per kg, of 941.5 g dry wheat middlings ground to pass a 0.8 μm screen, 50.0 g calcium carbonate, and 8.5 g manganese sulfate monohydrate. This exemplary formulation of wheat bran agar, or WBA, is used in examples below. Alternative formulations of WBA, especially where wheat bran media powder is included at lower concentrations, are specifically described where alternative formulations are used. Wheat bran agar media is a modification of semi-solid wheat media or SSWM that is described in detail at U.S. Pat. No. 10,138,444. As described in U.S. Pat. No. 10,138,444, “(the) composition of SSWM is 1,000 g of wheat bran along with 800 mL of deionized or distilled water and 200 mL of 0.1 M potassium phosphate buffer at pH 7.0, where 20 g calcium carbonate, 41 g calcium chloride, and 6.29 g manganese chloride tetrahydrate are suspended or dissolved in the liquid addition”. Example 1 demonstrates that, in a preferred embodiment, unlike TSA, sterility of WBA is not achieved by standard autoclave time for liquid media of 15 min at standard conditions of 121 degrees C. and 15 psi. Rather, extended autoclave time of 30 min, 45 min, or 50 min at standard conditions is suitable for sterilizing 9 percent WBA such that no contaminating microorganisms are detected by culture after 24 h incubation at 35 degrees C.

The outcome of culture procedures where media and surface sterility are achieved is the production of cultured plaque of strain 6A-1 that is free of contaminating bio-burden, as established by United States Pharmacopeia standards USP 41 and NF36, The United States Pharmacopeia and National Formulary Chapter 61. “Microbiological Examination of Nonsterile Products: Microbial Enumeration Tests.” And Chapter 62 “Microbiological Examination of Nonsterile Products: Tests for Specified Microorganisms.” (2018)) United States Pharmacopeial Convention, Inc. ISBN 978-3-7692-7022-8. Example 1 further documents that culture of strain 6A-1 on solid-phase media at commercial scale in a pan and lid assembly produces cultured plaque that is free of microbial contaminants, allowing for contaminant-free supplementation to animal feed.

Humidity and oxygenation in the contained culture atmosphere in an embodiment are controlled to also affect the outcome of solid phase culture, especially the total mass of cultured plaque of strain 6A-1 produced during the culture period. The effect of humidity is demonstrated in example 2 by circumstantial data that were obtained during an experimental incubation of strain 6A-1 on solid phase media in a vented (unsealed) pan and lid assembly. One pan of a triplicate treatment was found to have partially dried near the pan edge, which is a location in the pan that is most vulnerable to atmospheric exposure if the pan and lid assembly is vented rather than sealed. Plaque mass obtained from the partially dried pan was lower than in other pans found not to be partially dried. Therefore, exposure of the solid-phase culture surface to atmospheric conditions that are insufficiently humid, especially below 25 percent humidity, is projected to impair the yield of cultured plaque.

Previously noted in U.S. Pat. No. 10,138,444 is the ability of strain 6A-1 to vegetate and metabolize in fully aerobic conditions or in conditions of moderately decreased concentration of atmospheric oxygen, known as microaerophilic conditions. An embodiment provides the organism requires oxygen at lower levels than are present in the atmosphere (such as less than 21% oxygen). Control of atmospheric oxygen, if not necessary for maximizing cultured plaque output on solid phase culture or for maximizing a desired attribute within said cultured plaque, is a process that is not logistically or economically favorable for the method of solid phase culture. Data presented in example 3 demonstrate that a fully aerobic atmosphere or a microaerophilic atmosphere during solid phase culture resulted in similar mass of cultured 6A-1 plaque when tested on three types of solid phase media, which were minimal bacillus media or MBM (Demain, A. L. “Minimal media for quantitative studies with Bacillus subtilis.” J. Bacteriol, 75:517-522 (1958)), TSA, and WBA. The methods therefore encompass the use of aerobic or microaerophilic atmospheric conditions for the production of maximal cultured plaque mass of strain 6A-1.

The Method of Harvesting Plaque by Scraping from Solid Phase Media

In the method of the invention, culture on solid phase media in the conditions that have been described are projected as a novel means of producing cultured plaque of strain 6A-1. The method of plaque harvest by scraping cultured plaque of strain 6A-1 from solid phase media also is presented here as a novel method of cultured plaque production. The primary utility of solid-phase plaque production is the direct production of solid phase plaque and subsequent elimination of a centrifugation, filtration, or dehydration process that is otherwise required to separate cultured cells or cell products of said strain from a liquid phase culture. The method of solid phase culture is projected to decrease the costs of required infrastructure for the commercial production of strain 6A-1. A coincident utility of solid phase culture of strain 6A-1, demonstrated in example 4, is the increased harvest of cultured mass per bacterial cell by means of solid phase culture and scraping compared with the method of liquid phase culture and harvest by centrifugation. Greater harvested mass per bacterial cell is projected to result from increased harvest of bacterial exudates, or non-cellular mass, constituents of which are valuable for use as animal feed additives, which is elaborated further herein.

### The Method of Using Defined Culture Media and Conditions

Having identified and described numerous procedural variables in the method of the invention that are expected to affect or not affect the outcome of culturing strain 6A-1 on solid-phase media, the use of different culture media or modified culture media for producing maximal cultured plaque or variant compositions comprising strain 6A-1 and exudates of said strain is also disclosed herein. Four types of culture media are discussed further herein, especially MBM, TSA, and WBA, which have been presented previously herein, as well as liver-infusion tryptic soy agar, or LITSA, media, which is comprised per L of 25 g liver infusion broth powder (Difco™ Liver Infusion Broth, Becton Dickinson, Franklin Lakes, N.J. 07417), 25 g tryptic soy broth powder, and 25 g of granulated agar. Whereas MBM is a minimal media, TSA, LITSA, and WBA represent nutritive enrichments. Example 5 documents numerous tests whereby modifications of LITSA or WBA are shown to increase total plaque production. Specifically, media comprised of WBA brought to pH 7.0, or LITSA brought to pH between 6.0 and 8.0 with supplemental iron proteinate included at concentration of between 0.33 and 1.33 g per L, are shown to maximize the production of cultured plaque of strain 6A-1. Supplementation of iron proteinate to media other than LITSA also is projected to increase cultured plaque mass of strain 6A-1.

Wheat bran agar has been previously presented herein as a novel culture medium, whereas modifications to LITSA media are presented as novel on the basis of supplemental iron at alkaline pH. Inorganic sources of iron such is ferrous chloride, ferrous sulfate, or ferrous oxide are soluble in weakly acidic solutions, but ionized iron readily precipitates from solution as iron oxide upon neutralization of pH or adjustment of pH to greater than 7.0. Iron proteinate (Keyshure™ Iron, Balchem Corporation, New Hampton, N.Y. 10958) is an organically-complexed form of iron that is resistant to ionization in weakly acidic solution, so the structure of the organic complex is maintained as pH of solution is neutralized or made weakly alkaline. Therefore, supplementation of iron-proteinate to a neutral or weakly alkaline culture medium is a novel application with a utility that cannot be achieved by supplementation of inorganic iron sources.

### Culture Conditions for Increasing the Concentration of Cellulose-Degrading Bioactivity in Cultured Plaque of Strain 6A-1

The novel use of wheat bran agar, in addition to increasing production of total cultured plaque of strain 6A-1 on solid phase media, also increases the production of cellulose degrading bioactivity in said cultured plaque. Example 6 documents that plaque of strain 6A-1 has increased cellulose degrading bioactivity when cultured in either aerobic or microaerophilic conditions on WBA, especially at pH of 7.0 or 8.0, compared with other media known not to contain degradable polysaccharides. The inclusion of sources of naturally occurring or synthetic degradable fibers, other than wheat middlings, in culture media is projected to also increase the production of cellulose-degrading bioactivity in cultured plaque of strain 6A-1.

### The Minimum Constituent for Cellulose Degrading Bioactivity in Cultured Plaque of Strain 6A-1 is Protein CAB15943.1.

The composition comprising unrefined cultured plaque of strain 6A-1 has as a constituent the defined protein “endo-beta-1,3-1,4 glucanase, which is identified by GenBank accession number CAB15943.1 (SEQ ID NO: 4). Said protein is known to degrade cellulose. Protein CAB15943.1 is presented here as a minimum constituent of cultured 6A-1 plaque required for cellulose degradation. Example 7 documents the identification of said cellulose-degrading enzyme, as well as another polysaccharide-degrading enzyme identified as GenBank accession number CAB13776.1 (SEQ ID NO: 8), which is known to degrade beta-1,4 xylan, as exudates of strain 6A-1.

### Surface Active Emulsification Bioactivity in Cultured Plaque of Strain 6A-1

In the method of the invention, cultured plaque of strain 6A-1 has surface active emulsification (surfactant) bioactivity. Surfactant bioactivity in strain 6A-1 is enacted by a composition comprised of a non-polar phase extract of 6A-1, where exudate proteins of said strain identified as SEQ IDNO: 2 (CAB15086.1; “Biofilm-surface layer protein A”, or “biofilm hydrophobic layer component”) and SEQ ID NO: 3 (CAB15055.1; “Manganese binding lipoprotein”) are projected as the minimum constituents for enacting surfactant bioactivity. In the method here, surfactant bioactivity is quantified by colorimetric assay for methylene blue active substances.

Example 8 documents that surfactant bioactivity is enriched from cultured plaque of strain 6A-1 by solvent extraction of non-polar residues, and that said residues are quantified as methylene blue active substances, or MBAS, by colorimetric assay. Said residues are identified in example 9 as exudate proteins of strain 6A-1, especially proteins CAB15086.1 and CAB15055.1. Said non-polar residues refined by high performance liquid chromatography were subsequently shown to quantify as MBAS in Example 10 with greater potency per mol than sodium dodecyl sulfate, which is a known detergent. The utility of solvent-extracted non-polar residues as a feed additive for animals is elaborated further herein.

Solid phase culture conditions known to enrich cultured plaque for MBAS are comprised of culture in aerobic or microaerophilic conditions on TSA or LITSA media, especially where media is supplemented with a calcium salt. Example 11 documents a series of 5 experiments designed to test the effects of different basal media in aerobic or microaerophilic conditions, the effects of media pH, and the effects of supplemental calcium or iron on the concentration of MBAS in plaque. Notably, culture conditions that were observed to increase MBAS were generally observed to decrease cultured plaque mass, which was documented previously herein and in example 5.

The concentration of MBAS in cultured plaque of strain 6A-1 is presented as a surprising attribute of the strain. Example 12 documents a comparison of MBAS concentration in cultured plaque of Bacillus subtilis strains 6A-1, 168, and PB6. Bacillus subtilis strain 168 is a common laboratory reference strain, whereas Bacillus subtilis strain PB6 is a strain that is marketed commercially as an animal feed additive. Cultured plaque of strain 6A-1 was found to have approximately 8.5-fold greater concentration of MBAS in plaque than reference strain 168 and approximately 4.0-fold greater concentration of MBAS in plaque than commercial strain PB6. In embodiments the cultured plaque as described has at least one fold, two fold, three fold, four fold, five fold, six fold, seven fold, eight fold, nine fold, ten fold or more or amounts in-between higher that reference strain 168 and/or reference strain PB6.

### Utility of Cultured Plaque or Refined Plaque of Strain 6A-I as an Animal Feed Additive

Cultured plaque or refined plaque of strain 6A-1 is presented as a novel composition that has utility as a feed additive for animals. The direct supplementation of exudates of strain 6A-1 that have defined bioactivities comprised especially of cellulose-degrading bioactivity or surfactant bioactivity is distinguished here from the supplementation of spores or cells of strain 6A-1. Spores or cells of strain 6A-1 are projected to vegetate to a metabolically active state in the digestive tract and subsequently enact the bioactivities that have been described herein. Vegetation of supplemented spores or cells of strain 6A-1 in the digestive tract of an animal is not a process that is controlled during the manufacture of said spores or cells, so the enactment of said bioactivities in the digestive tract of an animal is projected to be equally not controlled by the supplementation of said spores or cells only. Comparatively, the manufacture of said bioactivities has been amply demonstrated herein by a process comprised of culturing strain 6A-1 on solid phase media and harvesting cultured plaque of said strain by scraping. Therefore, the delivery of said bioactivities to an animal by the method comprised of supplementing animal feed with cultured plaque of strain 6A-1 or refined plaque of said strain is novel. Methods for producing a composition comprising refined plaque of strain 6A-1 are elaborated further herein.

Surfactant activity enacted by methylene blue active substances in plaque of strain 6A-1, where supplemented as an additive to animal feed, is projected to increase the absorption or retention of dietary calcium and is projected to differently affect the absorption of particular dietary fatty acids. Said modes of action are projected to increase productive gains or productive outputs from food producing animals or are projected to increase the relative unsaturated fatty acid content (fat or fatty acid with at least one double bond in the fatty acid chain) among total fats or the total unsaturated fatty acid content of food produced from animals. Evidence for said modes of action comes from experiments in animals where animals were supplemented with spores of strain 6A-1 or refined plaque of strain 6A-1, where the observed effects in animals are linked to modes of action enacted by MBAS. The composition comprised of plaque or refined plaque of strain 6A-1, where surfactant bioactivity is quantified in said plaque as methylene blue active substances, is projected to increase the emulsification of dietary oils in digestive fluids, thereupon aiding the in vivo digestion of oil triglycerides into fatty acid constituents. Fatty acids in the digestive matter are thereupon projected to increasingly bind dietary calcium to form calcium salts of fatty acids, which are resistant to microbial metabolism in the foregut of ruminant animals and the gastrointestinal tract of ruminant or non-ruminant animals and are increasingly absorbed into animal tissue from the digestive matter or retained in animal tissue.

### Methylene Blue Active Substances Produced by Spores of Strain 6A-1 are Projected to Enact Increased Absorption of Calcium and Unsaturated Fatty Acids by Promoting the Formation of Calcium Soaps of Unsaturated Fatty Acids.

A series of 7 experiments in ruminant or non-ruminant animals documents that increased dietary calcium absorption or increased retention of calcium in tissues is induced by dietary supplementation of spores of strain 6A-1. Example 13 documents that when mature male sheep were supplemented or not supplemented with spores of strain 6A-1 in two consecutive experiments, apparent retention of dietary calcium in total body tissues was significantly increased, where apparent calcium retention was estimated by carefully measuring apparent absorption of dietary calcium and total urinary output of calcium. The two experiments presented in example 13 differed in the concentration of dietary oil that was fed to sheep, and increased apparent retention of dietary calcium was observed in sheep fed spores of 6A-1 regardless of the dietary oil inclusion rate.

Example 14 documents two experiments in which growing lambs that were supplemented with spores of strain 6A-1 were found to have greater concentration of calcium in liver tissue. Increased deposition of calcium in liver tissue is consistent with increased retention of absorbed calcium that was documented in example 13. Data provided in example 15 further document a statistical trend for lower calcium in manure of lactating cows housed on a commercial dairy farm, where cows were orally gavaged each day with a control solution or with spores of strain 6A-1. Decreased concentration of calcium in manure of cows treated with spores of strain supports that dietary calcium was increasingly absorbed.

Examples 16 and 17 present data from experiments conducted in non-ruminant animals. In example 16, refined plaque of strain 6A-1 was prepared by removing cells and spores by a series of centrifugation and filtration steps, and then biomass comprised of exudates of strain 6A-1 was either not supplemented or supplemented to growing mice in feed. Mice supplemented with refined plaque were found to have greater concentration of calcium in liver tissue, as was observed in ruminant studies presented in example 14. Importantly, this result was enacted by exudates of strain 6A-1, rather than by dietarily supplemented spores of said strain. Lastly, example 17 demonstrates that supplementation of spores of strain 6A-1 to growing pigs also increased the net retention of absorbed dietary calcium.

The observed increase in absorption or retention of dietary calcium supports that a mode of action is enacted by exudates of strain 6A-1 whereby calcium is made more available for absorption. As described previously herein, calcium in digestive fluids readily forms soap complexes with free fatty acids, where the solubility and absorbability of soap complexes comprised of unsaturated fatty acids, especially oleic acid or linoleic acid, is greater than for soap complexes comprised of saturated fatty acids, especially stearic or palmitic acid (Gacs, G. and Barltrop, D. “Significance of Ca-soap formation for calcium absorption in the rat.” Gut 18:64-68 (1977)). Unsaturated fatty acids in digestive fluids, especially rumen fluid in ruminant animals such as sheep or cattle, are subject to chemical biohydrogenation by resident microorganisms, but unsaturated fatty acid constituents of calcium soaps are resistant to biohydrogenation and are preserved as unsaturated fatty acids, rather than being hydrogenated to saturated fatty acids (Wu, Z., Ohajuruka, O. A., and Palmquist, D. L. “Ruminal synthesis, biohydrogenation, and digestibility of fatty acids by dairy cows. J. Dairy Sci. 74:3025-3034 (1991)). Therefore, where unsaturated fatty acids of triglycerides in dietary oils are digested to free fatty acids and biohydrogenated to saturated fatty acids, said saturated fatty acids are likely to form calcium soaps that are poorly available for absorption. However, where unsaturated fatty acids of triglycerides in dietary oils are rapidly digested to free fatty acids, the rate of calcium soap formation relative to the rate of biohydrogenation is expected to increase, and calcium soaps of unsaturated fatty acids are increasingly formed. Said soaps of unsaturated fatty acids are increasingly resistant to biohydrogenation, as previously discussed, and are therefore preserved with greater absorbability than calcium soaps of saturated fatty acids. The projected results of calcium soap formation with increased unsaturated fatty acid constituents are increased absorption of both unsaturated fatty acids and calcium as calcium soaps of unsaturated fatty acids are increasingly absorbed compared with calcium soaps of saturated fatty acids.

In the method of the invention, surface-active emulsifying bioactivity enacted by methylene blue active substances, comprised of proteins CAB15086.1 and CAB15055.1 as minimum constituents required for activity, is projected to increasingly emulsify dietary triglycerides and thereby hasten the formation of calcium soaps of unsaturated fatty acids, which are projected to be increasingly absorbed by animals fed spores, cells, cultured plaque, or refined plaque of strain 6A-1. Importantly, where animals are fed spores rather than cultured plaque or refined plaque of said strain, said spores are projected to vegetate and produce exudates comprising said surfactant bioactivity. Evidence is presented herein that animals supplemented with spores of strain 6A-1 develop altered composition of fatty acids in tissues, especially increased unsaturated fatty acids or decreased saturated fatty acids. Evidence relates especially to the production of food animal products such as meat and milk.

Example 18 documents that lactating dairy cows supplemented with spores of 6A-1 increasingly produce milk with a greater proportion of unsaturated fatty acids, especially oleic acid (18:1) and linoleic acid (18:2), in linear response according to increased dose of spores. Oleic acid and linoleic acid are the same fatty acids shown by Gacs and Barltrop to be absorbable in the intestine as calcium soaps (Gacs, G. and Barltrop, D. “Significance of Ca-soap formation for calcium absorption in the rat.” Gut 18:64-68 (1977)). Example 19 provides similar evidence that unsaturated fatty acids are increasingly accumulated in tissue of ruminant meat animals, whereas the saturated fatty acid palmitic acid is decreased as a proportion of total fatty acids.

### Manufacture of Refined Plaque of Strain 6A-1 for Use as an Animalfeed Additive

In the method of the invention, cultured plaque of strain 6A-1 is refined to retain bioactivities enacted by proteins CAB15943.1 (SEQ ID NO: 4; cellulose degrading bioactivity), or CAB13776.1 (SEQ ID NO8 5; xylan degrading bioactivity), or CAB15086.1 and CAB15055.1 (SEQ ID NO: 2 and SEQ ID NO: 6, respectively; surfactant bioactivity), or a combination thereof to be void of cells and spores of strain 6A-1. Example 20 documents a process whereby pilot-scale infrastructure was utilized to derive a composition comprised of exudates of cultured plaque of strain 6A-1 that was void of cells and spores of said strain. Recovery of exudates from plaque is shown to measure approximately 8.6 percent by mass. The same composition was documented in example 16 as an animal feed additive to mice with daily dose of equal to or less than 100 ng. Extrapolated on the basis of ng per g inclusion in animal feed, refined plaque of strain 6A-1 is projected to be supplemented to food-producing animals in complete feed at a rate of approximately 20 parts per billion, or 20 μg per kg. Increased rate of applied dosing is projected to enact improved animal performance on the basis of improved digestion of cellulose or xylan, or on the basis of improved emulsification of dietary oils. At a rate exceeding 0.1 percent inclusion, or 1 g per kg by mass in animal feed, supplementation of refined plaque is projected to exceed the normal inclusion rate for a micronutrient and is projected to be more costly or impractical as an animal feed additive.

Example 20 also documents the extraction of lipophilic or non-polar residues by non-polar phase extraction directly from plaque of strain 6A-1, where recovery of said residues from plaque was shown to measure approximately 0.6 percent by mass, compared with approximately 8.6 percent recovery of total exudate residues. In example 20, dry residues were produced by evaporating the respective solvent under heat, which was expected to cause a loss of surfactant bioactivity, so a measurement of surfactant activity for said residues is not provided. However, in the method of this embodiment, total plaque is aqueously solubilized, such as in water or water with a chemical buffer, and then is mixed with a solvent of lower polarity, whereupon the mixture is separated into solvent phases. The separated phase comprised of non-polar or organic solvent is projected to be enriched for surfactant activity, as extraction of surfactant activity by extraction into an organic solvent was demonstrated previously in examples 8, 9, and 10, collectively. Organic solvent is eliminated from residues by a drying process comprised of evaporation under heat or vacuum. Evaporation by heat is projected to increasingly result in loss of surfactant activity as constituent proteins are increasingly denatured.

Where surfactant activity quantified by methylene blue active substances as SDS equivalents is enacted by proteins CAB15086.1 or CAB15055.1, recovery of non-polar residues as approximately 0.6 percent of plaque by mass represents up to 24.8 percent recovery of projected total yield, considering that MBAS was measured in plaque at approximately 500 μg/g in similar plaque samples (Table 9) and the molar mass of protein CAB15086.1 (SEQ ID NO: 2) is 19.3 kDa compared with 288 kDa for SDS. (Said proteins refined by HPLC were estimated to be at least 1.36-fold more potent than SDS for MBAS per mol.) Therefore, modifications to solvent extraction procedures are projected to increase recovery of said residues. Projected modifications include the selection of any of the common organic solvents such as di-ethyl ether, petroleum ether, dichloromethane, hexane, pentane, or any of the common solvent alcohols such as methanol, ethanol, propanol, or butanol, or the selection of any combination of solvents for the utility of separation by polarity. Additionally, the inclusion of salt or chemical buffer to the aqueous phase during solvent separation procedures is projected to affect residue yields by means of a protein precipitation method commonly known as salting out, where proteins that are less polar in nature are increasingly lipophilic in an aqueous solvent with increased concentration of salts.

### DNA-Based Methods for Identifying Strain 6A-I as the Origin of Cultured Plaque or Refined Plaque.

Three examples are provided herein that document identification of strain 6A-1, cultured plaque of said strain, and refined plaque of said strain by a unique DNA sequence. Example 21 describes that DNA sequence 6360-1 (SEQ ID NO: 1) is unique to strain 6A-1 and is detectable by quantitative polymerase chain reaction. Examples 22 and 23 document the detection of sequence 6360-1 in cultured plaque and refined plaque, respectively. Where cultured plaque or refined plaque of strain 6A-1 is used as an animal feed additive, strain 6A-1 as the production source of said composition can therefore be identified.

### EXAMPLES

### Example 1

### Autoclave Time at Standard Conditions of 121 Degrees C. And 15 Psi for Achieving Culture Media Sterility is Dependent on Culture Media Type.

The objective of the present example is to demonstrate that, at standard autoclave conditions, the required time of autoclave exposure to achieve sterility of culture media is greater for wheat bran agar, or WBA, than for tryptic soy agar, or TSA, and that required autoclave time for sterility is increased for WBA comprised of 9 percent wheat bran media powder than for WBA comprised of lower inclusions of wheat bran media powder. Additionally, the culture of strain 6A-1 in metal pan and lid assemblies is demonstrated to be free of detectable bio-burden upon sterilization of media and surfaces by autoclave time of 25 min and 30 min, respectively, at standard autoclave conditions.

### Materials and Methods

In a series of experiments, TSA, WBA, or LITSA media were prepared, autoclaved, and poured into Petri plates or metal pan and lid assemblies and were incubated at 35 degrees C. for 12, 24, 48, or 72 hours. Data from all experiments are reported in Table 1. The temperature and pressure of all autoclave procedures were 121 degrees C. and 15 psi, respectively. In experiment 1, batches of culture media were prepared in 200 mL volume and then autoclaved for 15 min. The media that were tested were 1) TSA, comprised per L of 30 g tryptic soy broth (BD™ Tryptic Soy Broth, Becton Dickinson, Franklin Lakes, N.J. 07417) and 25 g granulated agar (Difco™ granulated agar, Becton Dickinson, Franklin Lakes, N.J. 07417), 2) WBA, 3 percent, comprised per L of 30 g wheat bran powder and 25 g granulated agar, where wheat bran powder was comprised, per kg, of 941.5 g dry wheat middlings ground to pass a 0.8 μm screen, 50.0 g calcium carbonate, and 8.5 g manganese sulfate monohydrate, 3) WBA, 6 percent, comprised per L of 60 g wheat bran powder and 25 g granulated agar, and 4) WBA, 9 percent, comprised per L of 90 g wheat bran powder and 25 g granulated agar. Upon autoclaving, media were tempered to a safe handling temperature of approximately 50 degrees C. and then were poured into duplicate Petri plates of 10 cm diameter. Petri plates were incubated aerobically at 35 degrees C. for 72 hours, and contaminant colonies were counted after 24 hours, 48 hours, and 72 hours of incubation. Data are reported as the average number of colonies from duplicate plates for each media treatment.

In experiment 2, WBA, 9 percent, was prepared in batches of 200 mL volume and autoclaved at standard conditions for 30 min, 45 min, or 50 min, and then media was tempered and poured into duplicate Petri plates as in experiment 1. Plates were incubated aerobically at 35 degrees C. for 12 h, 24 h, or 72 h. Data are reported as the average number of colonies from duplicate plates for each autoclave time treatment.

In experiment 3, liver-infusion trytic soy agar media, or LITSA media, was prepared in triplicate in 600 mL batches comprised per L of 25 g liver infusion powder (Difco™ Liver Infusion Broth, Becton Dickinson, Franklin Lakes, N.J. 07417), 25 g tryptic soy broth powder (BD™ Tryptic Soy Broth, Becton Dickinson, Franklin Lakes, N.J. 07417), and 25 g of granulated agar. Media was autoclaved for 25 min at standard conditions. Media was tempered and poured into pre-sterilized metal pan and lid assemblies, which were sterilized by autoclaving at standard conditions for 30 min. The culture surface was surface inoculated evenly with approximately 2.22×10⁹ spores of strain 6A-1 in 3 mL volume. Solid phase cultures were incubated at 35 degrees C. for 24 hours and then cultured plaque was obtained asceptically by scraping. Cultured plaque was analyzed for microbial bio-burden contaminants in accordance with United States Pharmacopeia standards 61 (USP 41-NF 36 chapter 61. “Microbiological Examination of Nonsterile Products: Microbial Enumeration Tests.” (2018)) and 62 (LSP 41-NF 36 chapter 62. “Microbiological Examination of Nonsterile Products: Tests for Specified Microorganisms.” (2018)). Enumeration of microbial contaminants in plaque produced by solid phase culture on pan and lid assemblies was below the limit of detection (less than 10 colony forming units per g) for all analyses in all samples.

### Results

Data shown in Table 1 document that TSA media in experiment 1 maintained sterility through 48 h of incubation by autoclaving time of 15 min at standard conditions. Similarly, 3 percent WBA, which was the lowest percentage of wheat bran media powder tested in WBA, maintained sterility through 72 hours. Higher inclusions in experiment 1 of wheat bran media powder in WBA were found not to be sterile through 24 hours or 48 hours and plates were overgrown with contaminant plaque after 72 hours of incubation. Upon autoclaving 9 percent WBA for 30 min, 45 min, or 50 min in experiment 2, media maintained sterility for 24 hours, whereas minor contamination was observed after 72 hours.

Bio-burden contaminants were not detected in experiment 3 where cultured plaque was produced at commercial scale in pan and lid assemblies. All analyses for the triplicate samples were below the limit of detection of 10 colony forming units per g plaque.

These data support that autoclaving time of 15 minutes is sufficient for sterilizing TSA culture media for use in 24 or 48 hour culture, whereas autoclave time of 30 min, 45 min, or 50 min is sufficient for sterilizing WBA with wheat bran media powder inclusion of up to 9 percent for use in 24 hour culture applications.

### Example 2

### Exposure of the Solid-Phase Culture Surface to Insufficient Atmospheric Humidity Decreases Production of Cultured Plaque of Strain 6A-1.

The objective of the present example is to demonstrate that insufficient atmospheric humidity can dry the culture medium and subsequently can decrease the total mass of cultured 6A-1 plaque produced during solid phase culture.

### Materials and Methods

An experimental solid phase culture of strain 6A-1 comprised of triplicate pan and lid assemblies was observed as part of a larger experiment. Solid phase media utilized for the culture was liver infusion tryptic soy agar (LITSA) comprised per L of 25 g liver infusion powder (Difco™ Liver Infusion Broth, Becton Dickinson, Franklin Lakes, N.J. 07417), 25 g tryptic soy broth powder (BDm Tryptic Soy Broth, Becton Dickinson, Franklin Lakes, N.J. 07417), and 25 g granulated agar. Media was adjusted to pH 7.0 by using 0.1 M hydrochloric acid and sterilized by autoclaving. Media was set to solid phase by pouring into 41.9 cm by 30.5 cm metal pans fit with removable lids as a pan and lid assembly. Media and pans were cooled at room temperature and surface inoculated with approximately 2.22×10⁹ spores of strain 6A-1 in 3 mL of diluent comprised of phosphate buffered saline. Pans were inverted horizontally and incubated at 35 degrees C. for 22 hours in an orbital shaker incubator (I2500 Series Incubator Shaker, New Brunswick Scientific, Enfield, Conn. 06082). The edges of the pan and lid assembly were vented rather than sealed, which allowed for atmospheric exposure to the culture surface. Humidity was measured in the orbital shaker incubator and in a standard laboratory incubator (CO₂ Incubator 605, Fisher Scientific, Waltham, Mass. 02451) for comparison, by a digital meter (AcuRite 00215CA, Chaney Instrument Co., Lake Geneva, Wis.).

### Results

Humidity in the orbital shaker incubator measured approximately 24 percent at 35 degrees C., compared with humidity of greater than 90 percent at 35 degrees C. in the standard laboratory incubator. Upon completion of culture procedures, the culture surface of a single pan of the triplicate cultures was found to have dried near an obvious area of increased atmospheric exposure. Growth of cultured plaque was visibly impaired on the dry area of the surface, compared with observations of previous cultures in a standard laboratory incubator. Cultured plaque mass obtained from the partially dried pan was 10.59 g, whereas uncompromised pans also cultured in the orbital shaker incubator produced cultured plaque mass of 11.93 g and 12.17 g, respectively. These data support that over-exposure of the solid-phase culture surface to insufficiently humid atmospheric conditions can cause the culture surface to dry and results in impaired cultured plaque production. Therefore, as atmospheric humidity is incrementally lower, especially below approximately 25 percent humidity, over exposure to atmospheric conditions is projected to impair plaque growth.

### Example 3

### Aerobic and Microaerophilic Atmospheric Conditions During Solid Phase Culture Result in Similar Mass of Cultured Plaque of Strain 6A-1 on Three Types of Culture Media.

The objective of the present example is to demonstrate that the use of aerobic or microaerophilic atmospheric conditions during solid phase culture results in similar production of cultured plaque of strain 6A-1.

### Materials and Methods

Atmospheric conditions comprised of unrestricted aerobicity or decreased oxygen concentration in a microaerophilic chamber were tested on three types of solid phase culture media, which were MBM (Demain, A. L. “Minimal media for quantitative studies with Bacillus subtilis.” J Bacteriol. 75:517-522 (1958)), tryptic soy agar or TSA, and wheat bran agar or WBA. Tryptic soy agar was comprised per L of 30 g tryptic soy broth powder (BD^{T}M Liver Infusion Broth, Becton Dickinson, Franklin Lakes, N.J. 07417) and 25 g granulated agar, whereas WBA was comprised per L of 90 g wheat bran powder and 25 g granulated agar, where wheat bran powder was comprised, per kg, of 941.5 g dry wheat middlings ground to pass a 0.8 μm screen, 50.0 g calcium carbonate, and 8.5 g manganese sulfate monohydrate. The pH of all media was adjusted to 7.0

Cultures were established on Petri plates (10 cm diameter), where the experimental unit was a stack of 3 Petri plates. Plates were surface inoculated with a pre-sterilized swab saturated with a solution of 6A-1 spores of approximate concentration 7.41×10⁸ spores per mL. Aerobic cultures for each media treatment were replicated over 3 stacks of plates, whereas microaerophilic cultures for each media treatment were replicated over 4 stacks of plates. Aerobic cultures were incubated in a standard laboratory incubator at 35 degrees C. without additional control of atmospheric exposure, whereas microaerophilic cultures were incubated in the same incubator in an air-tight chamber with decreased atmospheric oxygen concentration (BD GasPak® system using BD BBL™ CampyPak™ plus Microaerophilic System Envelopes with Palladium Catalyst (8801241) for determination. BD Becton, Dickinson and Company Sparks, Md. 21152 US.). Cultures were incubated for 24 hours and then cultured plaque was obtained by scraping. Mass of cultured plaque for a stack of 3 plates was measured by weighing. Mass of cultured plaque produced in aerobic or microaerophilic atmospheric conditions on each media was compared statistically by two-sample t-test. The effect of media type within each oxygenation condition was determined by completely randomized ANOVA, with means separated by the least significant difference test with a of 0.10. Data are reported in Table 2 as mean±SEM

### Results

Data presented in Table 2 demonstrate that no significant difference was observed for the production of cultured plaque mass with aerobic or microaerophilic atmospheric exposure. Data in Table 2 also demonstrate that plaque mass was significantly greater for culture on TSA media than on MBM media, and significantly greater for WBA media than for TSA media. The effect of media type was consistent in aerobic and microaerophilic atmospheric conditions.

### Example 4

Culture of Strain 6A-1 on Solid Phase Media and Harvest of Cultured Plaque of Said Strain by Scraping Increases the Production of Mass Per Bacterial Cell by Said Strain Compared with Liquid Phase Production Methods.

The objective of the present example is to demonstrate that culture of strain 6A-1 on solid phase media and harvest of cultured plaque of said strain by scraping results in the production of greater mass of plaque material per cell than is produced by liquid phase culture and harvest by centrifugation. The increase in plaque material per cell is projected to result from increased harvest of bacterial exudates, or non-cellular mass.

### Materials and Methods

Strain 6A-1 was cultured in triplicate in liquid phase media comprised of tryptic soy broth or on solid phase media comprised of tryptic soy agar, as previously described. Volume of both liquid phase and solid phase cultures was 20 mL, and the concentration of tryptic soy broth powder in liquid and solid phase cultures was equal. Cultures were incubated at 35 degrees C. for 24 h. Both solid and liquid phase cultures were incubated aerobically, where solid phase cultures were established in Petri dishes (10 cm diameter) and placed in a standard laboratory incubator and liquid phase cultures were established in culture tubes and placed in an orbital shaker incubator with 150 rpm oscillation to aerate the culture media. Upon completion of culture, the culture broth or plaque was sampled and the number of colony forming units of strain 6A-1 produced per culture was measured by means of serial dilution and re-culture. Additionally, cultured plaque produced by solid phase culture was obtained by scraping, whereas cells were pelleted by centrifugation (1,900×g for 10 min) from broth cultures. The mass of strain 6A-1 obtained from cultures was dried and weighed, and mass per colony forming unit was calculated. Data are reported in Table 3 as mean±SEM.

### Results

The number of cultured cells and the total microbial mass produced during culture were greater for liquid phase fermentation procedures than for solid phase fermentation, whereas microbial mass produced per cell was approximately 16 percent greater for solid phase than liquid phase fermentation. This result supports that plaque harvest by scraping from solid phase media compared with harvest by centrifugation from liquid media captures greater amounts of exudates per cell of strain 6A-1.

### Example 5

### Increased Mass of Cultured Plaque of Strain 6A-1 is Obtained on Novel Culture Media.

The objective of the present example is to demonstrate that the use of defined culture media is useful for producing maximal mass of cultured plaque of strain 6A-1 by means of aerobic solid phase culture. In the present example, a series of 10 culture experiments are summarized to demonstrate that cultured plaque output is affected by selection of a basal medium, by optimization of pH in culture media, and by enrichment of media with supplemental sources of calcium or iron.

### Materials and Methods

Mass of cultured 6A-1 plaque produced in a series of 10 experiments is shown in Table 4. For each experiment, Table 4 also describes the basal media that were tested, the final pH of test media, nutritive enrichments that were prepared into test media, the number of experimental units tested per treatment, and the duration of culture time in hours. Table 4 also lists the surface area in cm² for the experimental unit. Where metal pan and lid assemblies were used, the culture surface area was approximately 1,277 cm². Where Petri dishes were used for culture, an experimental unit comprised of a stack of 3 Petri dishes had a culture surface area of 235 cm² and a stack of 10 Petri dishes had a culture surface area of 785 cm². In all experiments, cultures were established as solid phase media and incubated aerobically at 35 degrees C. Measurements of plaque mass are presented as mean±SEM for all experiments except experiment 3, for which plaque mass is presented as the total mass produced on a stack of 10 plates.

### Results

The results of experiment 1 demonstrated that TSA, LITSA, or WBA media produce greater mass of cultured plaque than MBM media, where WBA produced the greatest plaque mass. Experiment was established in pan and lid assemblies, whereas experiment 2 was established in Petri dishes. Data from experiment 2 agree with data from experiment 1, where WBA was shown to produce the greatest plaque mass, and TSA and LITSA both produce greater plaque mass than MBM.

In experiment 3, TSA media was tested at pH of 4.5 to 7.5 in increments of 0.5 pH units, and the optimal pH for plaque production was found to be 7.0. Importantly, a functional limitation for media formulation was identified in experiment 3, where pH less than 5.5 caused the media to set fully in the solid phase. Therefore, in experiments 4 and 5, where optimal pH for maximizing plaque mass was tested in LITSA and WBA media, respectively, the minimum pH was limited to 6.0. In experiment 4, plaque production on LITSA media was not different on Petri dishes from pH 6.0 to 8.0, but comparison of culture on LITSA media in pan and lid assemblies at pH 7.0 or 8.0 in experiments 7, 8, 9, 10, and 11 documents that plaque production was consistently higher when strain 6A-1 was cultured at pH of 7.0 that at pH 8.0. Media pH of 7.0 was identified in experiment 5 as optimal for maximizing plaque production on WBA. In both experiments 4 and 5, strain 6A-1 was found to be markedly inhibited at pH 9.0 and was found not to grow at pH 10.0.

In experiments 6 and 7, iron proteinate (Keyshure™ Iron, Balchem) first was supplemented to LITSA media at titrated dose levels of 0.33 g/L, 0.66 g/L and 1.33 g/L in Petri dishes and found to increase plaque production at all levels tested compared with LITSA control media. Iron proteinate is comprised of 15 percent elemental iron by mass. Supplemental iron then was tested in pan-and-lid assembly format in experiment 7, and also found to numerically increase total plaque production. Importantly, the mass of plaque produced by LITSA control media in experiment 6 was similar to the mass of plaque produced by the same media in experiment 4. Experiments 6 and 7 were executed at pH 8.0 based on results from experiment 4.

In experiments 8 through 11, the effects of supplemental calcium sources were tested and were found to consistently decrease total plaque production. Calcium acetate at 125 mM (experiment 8), calcium chloride at 100 mM (experiment 9), and calcium nitrate at 100 mM (experiment 9) in culture media all were found to decrease plaque mass compared with respective controls. Plaque mass was further inhibited by calcium acetate at 250 mM (experiment 8) even compared with inclusion of calcium acetate at 125 mM. Calcium acetate dissociates in solution to one divalent calcium ions and two monovalent acetate ions, whereas sodium acetate dissociates in solution to one monovalent sodium ion and one monovalent acetate ion. Therefore, in experiment 8, sodium acetate was supplemented into media at 250 mM or 500 mM as a control against supplemental acetate originating from treatment with calcium acetate. Supplementation of media with sodium acetate resulted in decreased plaque production in similar fashion to supplemental calcium acetate, so the effect of supplemental calcium is not distinguished from the effect of supplemental acetate. These comparisons are shown in detail in Table 5, where calcium acetate treatments were compared with LITSA media by ANOVA and separation of means by Tukey HSD test with α of 0.05, and treatments comprised of equimolar acetate were compared by two sample t-test. In experiment 10 (data shown in Table 4), supplemental calcium chloride was titrated downward from 100 mM at 60 mM and 30 mM, and was found to inhibit plaque production even at concentration of 30 mM in media. Supplemental sodium chloride at concentration of 60 mM was tested in experiment 11 to demonstrate that the effect of impaired plaque growth is not the result of increased ionic strength, but rather the specific effect of a supplemental calcium salt. Supplemental sodium chloride was found not to inhibit plaque production in experiment 11. Data in experiments 8 through 11 support that supplemental salts of calcium to LITSA media are inhibitory to plaque growth.

The solid phase culture experiments of the present example demonstrate that strain 6A-1 can be cultured aerobically on WBA at pH of 7.0 for increased plaque output compared with other types of media. Production of plaque on LITSA media is optimized at pH 7.0, but is improved at pH of 8.0 with supplemental iron proteinate at concentration between 0.33 and 1.33 g per L.

### Example 6

### Plaque of Strain 6A-1 Cultured on Solid Phase Wheat Bran Agar Media at pH 7.0 has Increased Concentration of Cellulose-Degrading Bioactivity.

The objective of the present example is to document culture conditions that support increased production of cellulase bioactivity in cultured plaque of strain 6A-1. Data presented herein also support that a method comprised of diluting cultured plaque at into water at different dilution rates affects recovery of cellulase bioactivity among harvested exudates upon removal of cells by centrifugation. Two experiments are documented in the present example, where experiment 1 tested the effect of media type and atmospheric oxygenation and experiment 2 tested the effect of pH in WBA media in aerobic conditions.

### Materials and Methods

In experiment 1, strain 6A-1 was cultured aerobically on one of four media types, comprised of MBM, TSA, LITSA, or WBA, or in microaerophilic conditions comprised of MBM, TSA, or WBA. Cultures were established as triplicate stacks of three Petri plates each (10 cm diameter; 235 cm² surface area for three plates), where the stack of three plates was the experimental unit. Cultures were incubated for 24 hours at 35 degrees C. The pH of all test media was 7.0. Upon completion of culture procedures, plaque was obtained by scraping. To derive a sample from plaque suitable for measurement of cellulase activity, plaque was diluted approximately 500-fold in water, homogenized by shaking, and then centrifuged at 3,000×g for 30 min. The supernatant was passed through a 0.45 μm filter and then assayed for cellulase activity by procedures described in U.S. Pat. No. 10,138,444. Discussion from U.S. Pat. No. 10,138,444 pertinent to the procedure is as follows.

One unit of cellulase activity is defined as the quantity of enzyme that liberates 1 micromole of reducing sugar (expressed as glucose equivalents) per minute from the appropriate substrate under the conditions of the assay described. The cellulase substrate is sodium carboxymethyl cellulose. For the purposes of this assay substrate and cellulase enzymes reacted in 0.015 M Sodium Acetate Buffer, pH 5.0, prepared from sodium acetate trihydrate and acetic acid.

Glucose standards were prepared in deionized water and a standard curve was constructed for a range of glucose solutions from 0.1 mg/mL to 1.0 mg/mL. To each 0.8 mL of glucose dilution in a glass tube, 1.2 mL of DNS Reagent (1.0% 3,5-dinitro-salicylic acid solution (DNS) prepared in 0.4 N NaOH with 300 g/L of potassium sodium tartrate) was added. The tubes containing the standard glucose solutions were placed in boiling water bath for 10 minutes, after which they were cooled rapidly in ice water bath. 2.0 mL of deionized water was added to each tube. The reddish orange color developed by the DNS reagent in presence (of reducing sugar was read in suitable tubes in a spectrophotometer at 540 nm wavelength).

A 1.0% sodium (carboxymethylcellulose) . . . solution was prepared from low viscosity carboxymethyl cellulose sodium salt . . . . The substrate solution was prepared in . . . boiling 0.015 (M) acetate buffer.

The liquid enzyme-containing samples were analyzed in the following manner: to each tube containing 0.40 mL of 1% substrate solution was added 0.40 mL of enzyme containing solution. After mixing, tubes were incubated at 40.degree. C. for 30.0 minutes. After incubation, 1.2 mL of DNS was added to each tube. Tubes were subjected to a boiling water bath for 10.0 min after which they were immersed in an ice water bath and 2.0 mL of deionized water was added to each tube. The absorbance of each aliquot of reactant mixture was read in (a) spectrophotometer in suitable tubes at 540 nm wavelength.

The absorbance value for each enzyme-containing sample was calculated by subtracting the enzyme blank (absorbance) value from the enzyme sample (absorbance) value. The net value was used to calculate the activity value from the standard glucose curve.

In experiment 2, strain 6A-1 was cultured aerobically for 24 hours at 35 degrees C. in triplicate stacks of Petri plates (10 cm diameter, 235 cm² surface area for three plates) on WBA media. The pH of media was brought to 6.0, 7.0, 8.0, 9.0, or 10.0. Plaque growth obtained at pH 9.0 and 10.0 was insufficient for analysis. Total plaque obtained from each stack was diluted approximately 10-fold in water (compared with 500-fold in experiment 1), homogenized by shaking, and then centrifuged at 3,000×g for 30 min. The supernatant was retained and passed through a 0.45 μm filter, and then assayed for cellulase activity as in experiment 1.

Data from both experiments were respectively analyzed by completely randomized ANOVA. Means were separated by Tukey HSD test with a of 0.05. Data are presented as mean±SEM in Table 6.

### Results

Data from experiment 1 identify that plaque of strain 6A-1 produced by culture on solid phase media comprised of WBA was enriched for cellulase activity compared with plaque produced by culture on MBM, TSA, or LITSA media. This result was similar for aerobic and microaerophilic conditions. Data from experiment 2 identify that pH of 7.0 or 8.0 in WBA media increased the concentration of cellulase activity compared with pH 6.0 in media. At pH 9.0 or 10.0, the amount of plaque generated by culture was insufficient for analysis.

Notably, the preparative dilution of strain 6A-1 plaque into water for cellulase measurement was found to affect the harvest of cellulase bioactivity among total exudates. Data from both experiments were carefully evaluated to ensure that absorbance values produced in the assay were within the range of the standard curve. Indeed, in experiment 1, absorbance values produced for WBA media treatments were within range of the standard curve, whereas absorbance values produced for other media treatments were below the absorbance value produced by the lowest glucose standard. In experiment 2, all absorbance values that are reported also were well within range of the standard curve. Therefore, cellulase activity values reported in Table 6 for experiment 2 were measured and reported according to best practice, and no values reported for experiment 2 were below the limit of detection. The disparity in apparent concentration of cellulase bioactivity in plaque between experiments 1 and 2 where plaque was produced by culture on WBA is the result of solubilizing more total exudates of said plaque in the larger dilution volume of experiment 1. This result is consistent with observations that solubilized plaque is viscous in concentrated solution, such as the 10-fold dilution of experiment 2, whereas viscosity is observably decreased with greater dilution volume, such as the 500-fold dilution of experiment 1.

### Example 7

Identification of Cellulose Degrading Enzyme CAB15943.1 in B. subtilis 6A-1 Fermentation Product.

The objective of the present example is to provide identification of an enzyme that is responsible for cellulose degradation in B. subtilis 6A-1. U.S. Pat. No. 10,138,444 claims a method of producing carbohydrate degrading protein fractions by culturing 6A-1, wherein said protein fractions are capable of degrading crystalline cellulose, carboxymethyl cellulose, or unmodified cellulose. In the present example, we document the identification of a minimum constituent for the degradation of cellulose, which is protein CAB15943.1 (SEQ ID NO: 4), also known as “endo-beta-1,3-1,4 glucanase”.

### Materials and Methods

A fermentation product of B. subtilis 6A-1 was produced by culturing said strain on semi solid wheat media, which has been described in detail previously herein. The total fermentation product was dried and homogenized, and then a sample of the homogenate was reconstituted in water at a dilution of 1:10 (w/v). Total protein was precipitated from the homogenate with trichloroacetic acid, and then proteins were separated by two-dimensional gel electrophoresis using isoelectric focusing as the first dimension (7 cm Immobiline IEF strips, General Electric, Boston, Mass. 12345) followed by polyacrylamide gel electrophoresis in denaturing conditions with sodium dodecyl sulfate (SDS-PAGE). The gel containing separated proteins was stained and photographed (FIG. 1) using standard gel staining methods (Oriole™ gel stain, Bio-Rad, Hercules, Calif. 94547). Gel bands comprised of separated proteins were excised from the gel and digested with trypsin, and then trypsinized fragments were analyzed by liquid chromatography coupled to a Q-Exactive Plus mass spectrometer (Thermo Fisher Scientific, Waltham, Mass. 02451). Liquid chromatography procedures were executed using a Dionex Ultimate 3000 (Thermo Fisher Scientific, Waltham, Mass. 02451) system fitted with a Thermo Acclaim PepMap RSLC column (Thermo Fisher Scientific, Waltham, Mass. 02451). Solvents A and B were 0.1 percent formic acid in water and 0.1 percent formic acid in acetonitrile. Gradient (percent solvent B/minutes) was 1/0.00, 40/73.50, 95/90.00, 95/100.00, 1/100.10, 1/120.00. Spectra for trypsinized fragments were compared with predicted fragments generated from the genome sequence of strain 6A-1 to determine the protein identity of protein bands.

### Results

FIG. 1 documents 11 protein bands that were analyzed by mass spectrometry. Bands were selected for analysis on the basis of having weakly acidic to weakly neutral isoelectric point observed by two dimensional separation, because a composition comprising strain 6A-1 exudates of similar molecular character had previously been identified as having cellulose-degrading bioactivity (data not shown). The identity and molecular character of the 11 analyzed bands, as numbered in FIG. 1, is documented in Table 7. Gel band 7 was identified as protein CAB15943.1, or “endo-beta-1,3-1,4 glucanase”, which is known to confer cellulose degrading bioactivity. Additionally, gel band 10 was identified as protein CAB13776.1, or endo-1,4-beta-xylanase, which is known to confer xylan degrading bioactivity.

### Example 8

Enrichment of Surfactant Bioactivity from Cultured Plaque of Strain 6A-1 and Quantitation of Solvent-Extracted Non-Polar Residues as Methylene Blue Active Substances.

The objective of the present example is to demonstrate that surface active emulsification, or surfactant, bioactivity is extracted and enriched from cultured plaque of strain 6A-1 by means of solvent separation and harvest of non-polar residues, which subsequently quantify as methylene blue active substances, or MBAS.

### Materials and Methods

Strain 6A-1 was cultured overnight, aerobically, on solid phase LITSA media at pH 7.0 at 35 degrees C. for 24 hours. Upon completion of culture procedures, cultured plaque was obtained by scraping and a sample (approximately 1.0 g) was taken into water at approximate dilution of 1:300 (m/v). The resulting suspension was homogenized by shaking by hand for approximately 2 min. The homogenate was centrifuged four times at 3,000×g for 30 minutes, and the supernatant was consecutively retained. The supernatant was passed through a 0.45 μm filter, and then was concentrated by evaporation to a final volume of approximately 20 ml. The resulting solution was therefore comprised of crude exudates of strain 6A-1.

Crude exudates of strain 6A-1 were fractionated by solvent separation. An equal volume of dichloromethane was added, and the mixture was shaken vigorously by hand for approximately 1 minute. The mixture was then centrifuged at 1,500×g for 2 minutes. Separation of the two phases by centrifugation resulted in retention of the aqueous phase at the top of the centrifuge tube and derivation of the non-polar phase to the bottom of the centrifuge tube. The aqueous phase was harvested by pipetting, and the non-polar phase was retained separately.

Surface tension of the aqueous phase was measured directly, whereas the non-polar phase was first dried completely by evaporation under vacuum and then was reconstituted in a buffer comprised of tris(hydroxymethyl)aminomethane chloride, or Tris-Cl, at concentration of 20 mM. Surface tension was measured by a surface tension analyzer (Duran Wheaton Kimble, Milville, N.J. 08332) with a 0.5 mm internal diameter capillary tube, according to manufacturer's recommendations. Capillary height was measured to the nearest mm and was used to calculate surface tension according to the following equation (per manufacturer's specifications): y=(1/2)hrdg, where y=surface tension (dynes/cm), h=distance between basal volume meniscus and capillary meniscus (cm), r=radius of capillary (cm), d=density of sample (g/cm³ at room temperature), and g=acceleration due to gravity (cm/s²).

Surfactant bioactivity was separately measured as sodium dodecyl sulfate equivalents by colorimetric assay for MBAS. Methylene blue reagent (Sigma Aldrich, Saint Louis, Mo. 63103) was added to a constant volume of sample or standard (3.00 mL). Samples then were mixed with an equal volume of dichloromethane and separated to produce polar and non-polar fractions. Absorbance of the non-polar phase was read at 650 nm and compared with a standard curve produced by serial dilution of sodium dodecyl sulfate (Sigma Aldrich, Saint Louis, Mo. 63103). For all tests, samples were equilibrated into Tris-Cl buffer at pH 8.10, and Tris-Cl buffer served as a negative control for the assay.

### Results

Measurements of surface tension and MBAS for solutions comprised of aqueous or non-polar residues are shown in Table 8. Non-polar residues re-suspended in Tris-Cl buffer were found to have decreased surface tension and increased concentration of MBAS compared with control Tris-Cl buffer. Therefore, the composition comprised of non-polar, solvent-extracted residues that are quantified as methylene blue active substances from cultured plaque of strain 6A-1 enact surfactant bioactivity.

### Example 9

### Identification of Proteins CAB15086.1 and CAB15055.1 as Constituents of Solvent-Extracted Non-Polar Residues.

The objective of the present example is to identify the molecular constituents of an extract from plaque of strain 6A-1 in which surfactant bioactivity is observed. Two hydrophobic proteins identified as CAB15086.1 (“biofilm hydrophobic layer component”) and CAB15055.1 (“manganese binding lipoprotein”) were detected in the extract by liquid chromatography coupled with mass spectrometry.

### Materials and Methods

Strain 6A-1 was cultured aerobically overnight on solid phase LITSA media at 35 degrees C. Cultured plaque was obtained by scraping and diluted 10-fold into water, and then the suspension was homogenized by shaking. The homogenate was centrifuged at 3,000×g for 30 minutes and the supernatant was retained. The supernatant was extracted twice with an equal volume of diethyl ether. In each iteration of the extraction, diethyl ether was applied in equal volume to the aqueous homogenate, and then the mixture was shaken for 2 min by hand and centrifuged at 1,000×g. The supernatant was retrieved and retained. Diethyl ether was evaporated from solution under vacuum at room temperature. Approximate recovery of non-polar protein residues was 2.08 mg per g of cultured plaque.

Residues were subject to proteomic analysis by liquid chromatography coupled with mass spectrometry. Briefly, residues were brought into solution by addition of 30 percent (v/v) acetonitrile in water, and residues were digested with trypsin. Trypsinized fragments were analyzed by liquid chromatography coupled to a Q-Exactive Plus mass spectrometer (Thermo Fisher Scientific, Waltham, Mass. 02451). Liquid chromatography procedures were executed using a Dionex Ultimate 3000 (Thermo Fisher Scientific, Waltham, Mass. 02451) system fitted with a Thermo Acclaim PepMap RSLC column (Thermo Fisher Scientific, Waltham, Mass. 02451). Solvents A and B were 0.1 percent formic acid in water and 0.08 percent formic acid in 80 percent acetonitrile with water. Gradient (percent solvent B/minutes) was 3/2.00, 50/57.00, 99/70.00, 99/81.00, 2/81.10. Spectra for trypsinized fragments were compared with predicted fragments generated from the genome sequence of strain 6A-1 to determine the identity of constituent proteins in solution.

### Results

Proteins CAB15086.1 (“biofilm hydrophobic layer component”) and CAB15055.1 (“manganese binding lipoprotein”) were detected in the non-polar phase extract. Therefore, the composition comprised of non-polar, solvent-extracted residues known to enact surfactant bioactivity is comprised of proteins CAB15086.1 and CAB15055.1.

### Example 10

### Identification of HPLC-Fractionated Proteins as Methylene Blue Active Substances

The objective of the present example is to demonstrate that protein constituents of the composition comprised of non-polar residues extracted by solvent separation of cultured plaque of strain 6A-1 are quantified as potent methylene blue active substances (MBAS). The protein constituents of said composition are known to be comprised of proteins CAB15086.1 and CAB15055.1 as demonstrated previously herein.

### Materials and Methods

Cultured plaque of strain 6A-1 was produced by overnight aerobic culture on solid phase LITSA media at 35 degrees C. Cultured plaque was obtained by scraping and diluted 10-fold into water, and then the suspension was homogenized by shaking. The homogenate was centrifuged at 3,000×g for 30 minutes and the supernatant was retained. The supernatant was extracted twice with an equal volume of diethyl ether. In each iteration of the extraction, diethyl ether was applied in equal volume to the aqueous homogenate, and then the mixture was shaken for 2 min by hand and centrifuged at 1,000×g. The non-polar phase (upper phase in the centrifuge tube) was retained and total residues were obtained by evaporating the non-polar phase solvent at 70 degrees C.

Non-polar residues were reconstituted in a mixture of 30 percent methanol and 70 percent water. The sample was injected into a size exclusion chromatography column (BioSEC 5190-2513, Agilent Technologies, Santa Clara, Calif. 95051) on an Agilent 1260 Infinity II high performance liquid chromatography (HPLC) system using 20 mM Tris with 30% methanol as an isocratic mobile phase with detection 220 and 280 nm (indicative of peptide chemical bonds and aromatic amino acids, respectively). A dominant protein fraction detected at both wavelengths was collected in-line during the separation. The eluted protein fraction was re-extracted from HPLC buffer with diethyl ether and then the solvent was evaporated and residues were reconstituted in 3 mL water. Residues were assayed for MBAS as sodium dodecyl sulfate equivalents, or SDS equivalents, by procedures described previously herein.

### Results

The concentration of MBAS in the assay measured approximately 5.4 μg SDS equivalents per mL for estimated total recovery of 16.2 ug SDS equivalents. The total dry mass of MBAS residues, which were derived during the MBAS assay, was obtained by weighing after evaporating the assay fraction containing MBAS to dryness in a container of known mass. Total dry mass of MBAS measured approximately 0.8 mg, so SDS equivalents per mg residue measured approximately 20.3 μg per mg. Proteins CAB15086.1 and CAB15055.1 have estimated molar mass of approximately 19.3 kDa and 33.4 kDa, respectively, compared with molar mass of SDS of 288 Da. Therefore, surfactant bioactivity per mol of HPLC-refined protein comprised of CAB15086.1 and CAB15055.1 was approximately 1.36-fold to 2.35-fold greater than the surfactant activity of SDS.

### Example 11

Methylene Blue Active Substances are Enriched in Plaque of Strain 6A-1 by Culturing on TSA or LITSA Media with Supplemental Calcium Salts.

The objective of the present example is to document that MBAS is enriched in cultured plaque of 6A-1 where said plaque is cultured on TSA or LITSA media, especially where calcium salts are supplemented to media. A series of 5 culture experiments that are described in Table 9 was produced to compare the effects of different media on MBAS concentration in cultured 6A-1 plaque in aerobic or microaerophilic conditions and the effects of media pH, supplemental calcium salts, and supplemental iron proteinate.

### Materials and Methods

In experiment 1, media comprised of solid phase MBM, TSA, LITSA, or WBA at pH 7.0 were tested in Petri dishes for effects on MBAS concentration in plaque of strain 6A-1 cultured on said media. Cultures were incubated aerobically for 24 hours at 35 degrees C. Upon completion of culture procedures, plaque was obtained by scraping and diluted approximately 500-fold into water. Methylene blue active substances were measured by colorimetric assay procedures previously described herein. Experiment 2 was established like experiment 1, but media comprised of MBM, TSA, or WBA were tested in microaerophilic conditions.

Experiment 3 was established to test the effects of media pH. LITSA media was selected for this test because the mass of cultured plaque was previously shown to be greater for LITSA than for TSA, as demonstrated previously herein in experiments 1 and 2 of table 4. Cultures of strain 6A-1 were established as in experiment 1, but media pH was adjusted to 6.0, 7.0, 8.0, 9.0, or 10.0. Cultured plaque was obtained by scraping, diluted approximately 200-fold into water, and assayed for MBAS concentration as in experiments 1 and 2. Cultures established at pH 10.0 produced insufficient mass for analysis of MBAS concentration.

Experiments 4 and 5 were established in pan and lid assemblies and were cultured aerobically for 22 hours at 35 degrees C. Two different calcium salts (calcium chloride or calcium nitrate) were tested in LITSA media at pH 7.0 in experiment 4, whereas supplemental iron proteinate was tested at 1.33 g/L in LITSA media at pH 8.0 in experiment 5. The cultures of experiments 4 and 5 in the present example are the same respective cultures of experiments 9 and 7 presented in table 4. As in experiments 1 through 3 of the present example, cultured plaque was obtained by scraping upon completion of culture procedures. Plaque was diluted approximately 350-fold into water and then assayed for MBAS concentration.

Data from experiments 1 through 5 are reported in Table 9 as means SEM. Experiments 1 through 4 were analyzed by completely randomized ANOVA and means were separated by Tukey HSD test with a of 0.05. Data from experiment 5 was analyzed by two sample t-test with significance reported at P<0.05.

### Results

Data from experiments 1 and 2 document that concentration of MBAS in cultured plaque was generally greater for TSA or LITSA media than for MBM or WBA. In experiment 3, pH of 6.0, 7.0, or 8.0 was shown not to affect concentration of MBAS in plaque, whereas pH 9.0 decreased the concentration of MBAS in cultured plaque. Supplemental calcium salts significantly increased the concentration of MBAS in cultured plaque, whereas no effect was observed for supplemental iron proteinate. These data support that culture of strain 6A-1 on solid phase media comprised of TSA or LITSA with supplemental calcium salts is enriched for concentration of MBAS.

### Example 12

Concentration of MBAS in Cultured Plaque of Strain 6A-1 is Greater than for Reference Strain Bacillus subtilis Strain 168 and Commercial Strain Bacillus subtilis Strain PB6.

The objective of the present example is to demonstrate that strain 6A-1 produces a surprising concentration of MBAS in cultured plaque compared with other strains of Bacillus subtilis.

### Materials and Methods

Bacillus subtilis strains 6A-1, 168, and PB6 were cultured experimentally. Strain 168 is the common, research strain of Bacillus subtilis and was obtained from the American Type Culture Collection (ATCC 23857). Strain PB6 is a known commercial strain of Bacillus subtilis (Kemin Industries, Des Moines, Iowa 50317). Cultures were established on solid phase brain heart infusion, or BHI, media comprised per L of 37 g brain heart infusion powder (BBL™ Brain Heart Infusion, Becton Dickinson, Franklin Lakes, N.J. 07417) and 25 g granulated agar. Three cultures per strain were established on Petri plates, where 1 plate per media treatment was brought to one of three pH treatments, comprised of pH 5.5, 6.0 or 6.5. Cultures were incubated at 35 degrees C. for 24 hours. Upon completion of culture procedures, cultured plaque was obtained by scraping and was diluted 10-fold into water and homogenized by vortexing. Plaque dilutions were rested at 4 degrees C. for 18 hours, and then were centrifuged at 10,000×g for 15 min. The supernatant was retained for measurement of MBAS by procedures described previously herein. Data were analyzed by ANOVA, where the statistical model was Yᵢⱼ=μ+Sᵢ+Pⱼ+eᵢⱼ, where Sᵢ was bacterial strain (i=3), Pⱼ was pH (j=3), and eᵢⱼ was the residual error. Means were separated by Tukey HSD test with a of 0.05. The effect of media pH was not significant. Data are presented as mean±SEM in Table 10 for the effect of bacterial strain.

### Results

Data in Table 10 document that concentration of MBAS was significantly greater for strain 6A-1 than for Bacillus subtilis strains 168 or PB6. Cultured plaque of strain 6A-1 was found to have approximately 8.5-fold greater concentration of MBAS in plaque than reference strain 168 and approximately 4.0-fold greater concentration of MBAS in plaque than commercial strain PB6. Therefore, cultured plaque of strain 6A-1 is surprisingly enriched for MBAS.

### Example 13

### Dietary Supplementation of Spores of Strain 6A-1 to Sheep Increases Net Uptake and Retention of Dietary Calcium.

The objective of the present example is to demonstrate that dietary supplementation of B. subtilis 6A-1 spores as a feed additive to sheep increases the net absorption and retention of dietary calcium. The present example is comprised of two experiment in which sheep were supplemented or not supplemented with spores of strain 6A-1. The two experiments utilized different concentrations of dietary oil, which were approximately 3.0% oil in experiment 1 and approximately 5.0% oil in experiment 2.

### Materials and Methods

Adult male sheep (n=23) were utilized in two consecutive experiments. In experiment 1, sheep were fed a diet of approximately 3.0% oil on a dry matter basis and were randomly assigned to treatments of daily oral gavage with a control solution (dextrose carrier) or with B. subtilis 6A-1 spores in dextrose carrier to provide approximately 2.0×10⁶ spores per lb of dry matter feed intake, or approximately 2.6×10⁷ spores per d. In experiment 2, sheep were fed a high-fat diet of approximately 5.0% oil and treated as in experiment 1. The diet in both experiments was comprised of corn silage, alfalfa haylage, distillers grains, soybean meal, and a vitamin and trace mineral supplement formulated to meet or exceed nutritional requirements. Diets were formulated with a similar inclusion of dietary calcium, which was approximately 0.73 percent on a dry matter basis.

Each experiment was executed as a switch-back design with two periods, where the sheep treated as control in period 1 were treated with spores in period 2, and vice versa. Each period within experiments 1 and 2 consisted of 20 days. On days 1-20, sheep were fed the basal diet and were orally gavaged daily with the appropriate treatment. On day 14, sheep were placed into individual digestibility crates to begin a 3-day crate adaptation period. On days 17 through 19, the mass of total feed delivered to each sheep was recorded. On days 18-20, the mass of total feed refused, the mass of manure produced in the most recent 24 hours, and the mass of urine produced in the most recent 24 hours, were recorded. Samples of each material, per sheep, were retained for chemical analysis. Calcium was measured by AOAC method 985.01 ([AOAC] Association of Official Analytical Chemists. “Metals and other elements in plants and pet foods: inductively coupled plasma spectroscopic method, AOAC official method 985.01.” (2003)). Total excreted calcium was calculated by multiplying the concentration of calcium in urine by the mass of urine obtained. Apparent absorbed calcium was calculated by subtracting total calcium excreted in manure from total dietary calcium consumed. The ratio of milligrams of excreted calcium per grams of apparent absorbed calcium was then determined, where a lower ratio describes greater retention of absorbed calcium in tissues.

Data were summarized by treatment for each experiment and are presented as mean SEM in Table 11. The difference between measurements obtained for control and treatment periods was determined for each sheep, and the difference was analyzed by one-sample t-test. The individual sheep served as the experimental unit. Statistical significance was established at P<0.05.

### Results

Data presented in Table 11 document that in both experiments, excreted calcium was numerically lower and apparent absorbed calcium was numerically higher for sheep treated with spores than for sheep treated as control. The ratio of excreted calcium per apparent absorbed calcium was significantly lower for sheep treated with spores than for control in both experiments. Therefore, spores of strain 6A-1 increased the retention of absorbed calcium, and the result was not different on the basis of dietary oil inclusion.

### Example 14

### Dietary Supplementation of Spores of Strain 6A-1 to Growing Lambs Increases Deposition of Calcium in Liver Tissue.

The objective of the present example is to demonstrate that supplementation of spores of strain 6A-1 to growing lambs increases the deposition of calcium in liver tissue.

### Materials and Methods

Two experiments are presented herein, where sheep in experiment 1 were either not supplemented (n=11) or supplemented (n=15) with spores at a rate of approximately 1.4×10¹⁰ spores per lb. of dry matter feed intake, and sheep in experiment 2 were either not supplemented (n=16) or supplemented (n=15) with spores at a rate of approximately 1.1×10¹⁰ spores per lb. of dry matter feed intake. The diet in both experiments was comprised of corn silage, alfalfa haylage, distillers grains, soybean meal, and a vitamin and trace mineral supplement formulated to meet or exceed nutritional requirements. The diet in experiment 1 was comprised on a dry matter basis of approximately 30 percent crude fiber, 13 percent crude protein, 3.4 percent oil, 31 percent starch, and 0.99 percent calcium. The diet in experiment 2 was comprised on a dry matter basis of approximately 37 percent crude fiber, 12 percent crude protein, 3.2 percent oil, 23 percent starch, and 1.16 percent calcium. The duration of treatment was 93 days in experiment 1 and 96 days in experiment 2.

Upon completion of the feeding period, lambs were euthanized and liver tissue was collected. Liver tissue was dried in a 70 degree dryer and homogenized by grinding, and then calcium was measured in dry material by AOAC method 985.01. The concentration of calcium in liver tissue was compared between control and spore-treated groups within each experiment by two-sample t-test. Data are presented as mean±SEM with the associated P value in table 12. Statistical significance was established at P<0.05.

### Results

Data shown in Table 12 document that concentration of calcium in liver tissue was significantly greater in experiment 1 for lambs supplemented with 6A-1 spores than for lambs not supplemented with spores. In experiment 2, lambs supplemented with 6A-1 spores had numerically higher concentration of calcium in liver tissue compared with the control.

### Example 15

### Supplementation of Spores of Strain 6A-1 to Lactating Dairy Cows Decreases the Concentration of Calcium in Fecal Matter.

The objective of the present example is to demonstrate that supplementation of spores of strain 6A-1 to lactating dairy cows decreases the concentration of calcium in fecal matter. Decreased concentration of calcium in manure relates to increased absorption of dietary calcium.

### Materials and Methods

Lactating dairy cows were housed on a commercial dairy farm and fed a total mixed ration. The ration was formulated for approximately 30 percent crude fiber, 16 percent crude protein, 5.0 percent oil, 26 percent starch, and 1.04 percent calcium. Beginning on the first day after parturition, cows received one of two treatments daily for 28 consecutive days, which were oral gavage with glucose solution (n=6), or oral gavage with glucose solution containing approximately 1.0×10⁹ spores of strain 6A-1 (n=5). The concentration of glucose in oral gavage solution was approximately 20 g per L and cows were dosed with approximately 20 mL of solution each day. Samples of fecal material were collected rectally on d 28. Fecal material was dried and then analyzed for calcium by AOAC method 985.01. Concentration of calcium in manure was compared between control and spore-treated groups by two-sample t-test. Statistical significance was considered at P<0.05 and a statistical trend was considered at P<0.10.

### Results

The concentration of calcium in manure for control and spore-treated groups is shown in Table 13. A statistical trend was observed for lower concentration of calcium in fecal matter of animals treated with spores of 6A-1.

### Example 16

### Supplementation of Refined Plaque of Strain 6A-1 Increases Deposition of Calcium in Liver Tissue of Mice.

The objective of the present example is to demonstrate that supplementation of refined plaque of strain 6A-1 to mice causes increased deposition of calcium in liver tissue. Said refined plaque was void of cells and spores of said strain, so increased deposition of calcium in liver tissue was a result enacted by the exudates of strain 6A-1.

### Materials and Methods

Refined plaque of strain 6A-1 was manufactured to be void of cells and spores of said strain. Strain 6A-1 was cultured on LITSA media aerobically at pH of 7.0 and cultured plaque of said strain was obtained by scraping. Cultured plaque was diluted 500-fold into water and homogenized by shaking. Said diluted homogenate was passed through a 0.1 μm filter and sterility of said filtrate was verified by culture. The filtrate was concentrated by lyophilizing to a dry powder, and then a known mass was reconstituted in water and applied to mouse feed by spraying during feed manufacture.

White laboratory mice (n=15 per treatment) were utilized to test the effects of dietary treatments comprised of control, which was not supplemented with refined plaque of strain 6A-1, or supplemental refined plaque of strain 6A-1 applied at daily doses of 10, 25, 50, or 100 ng per mouse, which were designated as treatments RP10, RP25, RP50, or RP100, respectively. Treatments were administered for 33 days and then mice were euthanized for collection of liver tissue. Calcium was measured in dried liver tissue by AOAC method 985.01.

Concentration of calcium in liver tissue was logarithmically normalized (log₁₀) to achieve a normal distribution of data. Log-transformed data were analyzed by completely randomized ANOVA for the effect of dietary treatment. An orthogonal contrast statement was used to separate means between the control group and all groups supplemented with refined plaque. Data are presented as mean±SEM in Table 14. Statistical significance is presented at P<0.05 and a statistical trend is presented at P<0.10.

### Results

Data presented in Table 14 demonstrate that although no single treatment mean for groups supplemented with refined plaque was significantly different from the control (P=0.250), the use of orthogonal contrast to make an aggregate comparison between the control group and groups supplemented with refined plaque identified a statistical trend (P=0.097) where mice supplemented with refined plaque were found to have increased concentration of calcium in liver tissue. Because refined plaque was void of cells or spores of strain 6A-1, increased deposition of calcium in liver tissue is discerned to be a result enacted by the exudates of strain 6A-1 that are retained in refined plaque.

### Example 17

### Supplementation of Spores of Strain 6A-1 to Swine Increases the Net Retention of Absorbed Dietary Calcium.

The objective of the present example is to demonstrate that supplementation of spores of strain 6A-1 to swine increases the net retention of absorbed dietary calcium. This finding is meaningful because the digestive system of swine (non-ruminant) is anatomically different from that of ruminant species such as sheep and cattle, for which similar findings have been documented previously herein.

### Materials and Methods

Growing pigs (n=12 per treatment) were randomly assigned to one of two treatment groups, which were not supplemented (control) or supplemented with spores of strain 6A-1 (6A-1) at a rate of approximately 1.8×10⁸ spores per lb. of complete feed on an as-fed basis. Samples of urine and fecal matter were obtained at 12 weeks of age. Calcium and aluminum were measured on a dry basis in samples of feed and fecal matter by AOAC method 985.01. Calcium and creatinine were measured in urine by AOAC method 985.01 and by colorimetric assay, respectively. For measurement of creatinine, 1.0 mL of 1 percent picric acid, 75 μL of 10 percent sodium hydroxide were added to 10.0 μL of urine or serially diluted creatinine standard (Sigma Aldrich, Saint Louis, Mo. 63103) in a cuvette and incubated for 10 min. Water (3.75 mL) was added to the mixture, and then absorbance was read at wavelength of 520 nm.

Calcium digestibility was determined by calculating the difference between the ratio of calcium and aluminum in feed and fecal matter. The concentration of urinary calcium was normalized to the concentration of creatinine, and then the ratio was logarithmically transformed (log₁₀) to achieve a normal distribution of data. Normalized concentration of calcium in urine was expressed as a ratio with estimated calcium digestibility and multiplied by 1,000 to approximate urinary calcium excretion relative to estimated calcium digestibility, where a lower value describes increased retention of absorbed calcium. Data were analyzed by two-sample t-test and are reported as mean±SEM in Table 15.

### Results

Urinary excretion of calcium normalized to calcium digestibility was significantly lower for pigs treated with spores of strain 6A-1, which indicates greater retention of absorbed calcium. Calcium digestibility was approximately equal between control and spore-treated animals, whereas normalized concentration of calcium in urine was numerically lower for spore-treated animals by approximately 12.1 percent. These results demonstrate that absorbed calcium was increasingly retained in animals supplemented with spores of strain 6A-1.

### Example 18

### Supplementation of Spores of Strain 6A-1 to Lactating Dairy Cows Enacts a Dose-Dependent Response in the Proportion of Unsaturated Fatty Acids in Milk.

In the present example, the results of an experiment show that feeding spores of Bacillus subtilis 6A-1 to lactating dairy cows causes an increase in the unsaturated fatty acid content of dairy cow milk.

### Materials and Methods

The experiment was designed as a complete block design with 5 treatments replicated over 5 blocks. Multiparous, lactating cows of Holstein or crossbreed influence were assigned randomly to a treatment by order of parturition within each block. Treatments were daily oral gavage for the first 28 d post-calving with approximately 0.0×10⁰ (control), 1×10⁶, 1×10⁷, 1×10⁸, or 1×10⁹ colony forming units of Bacillus subtilis, subsp. subtilis strain 6A-1. Cows were housed on a commercial dairy farm and fed a commercial total mixed ration formulated to meet or exceed nutritional requirements.

A metered milk sample was collected from cows in the morning on approximately d 21 post-calving. Milk fat was analyzed for fatty acid constituents by the fatty acid methyl ester (FAME) method. Briefly, milk was dried at 45 degrees C. and then approximately 250 mg of dry material was mixed with 2.0 mL toluene, 1.0 mL acetone, and 5.0 mL methanol with 2.0 percent sulfuric acid (v/v). Tridecanoic acid (13:0) was used as an internal standard. The mixture was incubated at 70 degrees C. for 2 h, cooled in an ice bath for 20 min, and then mixed with 1.0 mL hexane and 5.0 mL of 6 percent (w/v) potassium carbonate. The mixture was separated by centrifugation at 500×g for 10 min and the organic phase was retained. The solution was clarified by adding 100 mg activated charcoal (100 mg) and 200 mg sodium sulfate followed by re-centrifugation. The supernatant was analyzed by gas chromatography (7890A GC system, Agilent Technologies, Santa Clara, Calif.).

Data were analyzed by complete block analysis of variance (ANOVA) and a polynomial contrast statement was used to detect linear effects of treatment. A significant linear effect (P<0.05) or a significant linear trend (P<0.10) indicates that the biological response was exemplified with incrementally increased levels of the applied treatment. In the present example, the applied increment was an exponential (10-fold) increase of supplemental 6A-1 spores. The proportion of saturated fatty acids, unsaturated fatty acids, mon-unsaturated fatty acids and poly-unsaturated fatty acids, as well palmitic acid (16:0), oleic acid (18:1) and linoleic acid (18:2) are presented in Table 16 as mean±SEM.

### Results

Results presented in Table 16 demonstrate that the proportion of unsaturated fatty acids (and subsequently the proportion of saturated fatty acids) was significantly dose responsive (P=0.029). The proportion of oleic acid (18:1), which is the primary mono-unsaturated fatty acid in milk and prevalent among dietary fatty acids, also was significantly dose responsive and increased with increasing spore dose. Linoleic acid (18:2), which is also a principal constituent of dietary fatty acids, was dose responsive as a statistical trend (P<0.10). These results demonstrate that supplementation of 6A-1 spores to dairy cows increasingly results in the production of dietary unsaturated fatty acids into milk fat.

### Example 19

### Supplementation of Spores of Strain 6A-1 to Beef Feeder Animals Decreases the Proportion of Saturated Fatty Acid Palmitic Acid and Increases the Proportion of Unsaturated Fatty Acid Elaidic Acid in Ribeye and Fat Tissue

The objective of the present example is to demonstrate that supplementing spores of strain 6A-1 to growing market steers causes the production of a food product with altered fatty acid composition. Specifically, intramuscular fat from the ribeye contains a lower proportion of palmitic acid (a saturated fatty acid; 16:0) and a greater proportion of elaidic acid (an unsaturated fatty acid; 18:1). Adipose tissue obtained from animals also was found to have a significantly lower proportion of palmitic acid among total fatty acids.

### Materials and Methods

Two groups of beef feeder animals (n=6 per group; 3 steers and 3 heifers per group; Holstein×Wagyu crossbreed) were experimentally treated as control or supplemented with 6A-1 spores at a rate of 1×10⁷ spores per d. Steers were fed standard feeder and finisher diets until the time of slaughter. Samples of ribeye muscle tissue and adipose tissue from backfat were analyzed by the fatty acid methyl ester method, as described previously to determine fatty acid composition. All metrics were analyzed by two-sample t-test. Data are reported as mean SEM.

### Results

Results from fatty acid methyl ester analysis are reported in Table 17. In ribeye tissue, the percentage of palmitic acid among total fatty acids was significantly decreased (P=0.011) in animals supplemented with 6A-1, whereas the percentage of elaidic acid (18:1) tended to be greater (P=0.081). In adipose tissue, the percentage of palmitic acid among total fatty acids also was significantly decreased (P=0.010).

### Example 20

### Cultured Plaque of Strain 6A-1 is Refined to a Composition Comprised of Exudates of Said Strain or a Non-Polar Phase Extract of Said Strain or Exudates.

The objective of the present example is to document that methods for the refinement of cultured plaque to a composition comprised of exudates of strain 6A-1 but not cells or spores of said strain increases the concentration of methylene blue active substances in said refined composition. In the present example, total exudate yield and MBAS concentration in refined plaque is documented for cultured plaque produced on solid phase MBM, TSA, LITSA, or WBA media, where cultured plaque was produced in pan and lid assemblies. Separately, yield of exudates obtained by solvent extraction procedures also is documented.

### Materials and Methods

Strain 6A-1 was cultured aerobically for 24 hours at 35 degrees C. on solid phase MBM, TSA, LITSA, or WBA media. All media were brought to pH 7.0 and prepared in pan and lid assemblies in duplicate. The culture surface was inoculated with approximately 2.22×10⁹ spores in 3 mL volume. These cultures are the same as presented in experiment 1 of Table 4, and refinement procedures enacted on these plaque samples are presented in Table 18 as refinement set 1. Total plaque was obtained by scraping, diluted 100-fold (w/v) in water, and was homogenized by shaking. The homogenate was centrifuged at 3,000×g for 30 min and the supernatant was consecutively passed through a 0.45 m and 0.10 m bottletop filters (Whatman, General Electric, Boston, Mass. 12345). The filtrate was lyophilized and resulting mass was measured by weighing. Concentration of methylene blue active substances was measured in exudates by procedures that have been described previously herein. Data are recorded as mean±SEM in Table 18. Data were analyzed statistically by completely randomized ANOVA, where pan was the experimental unit. Means were separated by the Least Significant Difference test with a of 0.10.

Separately, refinement procedures presented in Table 18 as refinement set 2 were enacted by means of solvent extraction, whereby cultured plaque of strain 6A-1, cultured aerobically for 22 hours on LITSA media at pH 7.0, was diluted 20-fold (w/v) into water and then extracted repeatedly with petroleum ether. With each iteration of the extraction, petroleum ether was applied in equal volume to the aqueous suspension and then the suspension was homogenized by shaking by hand for 1 min. The homogenate was then separated by centrifugation at 500×g for 2 min and the non-polar or organic phase was retained. The extraction was iterated 5 times. The organic phase was obtained as a gel in early iterations of the extraction, whereas in later iterations the organic phase was a liquid, where residues obviously protruding into the organic phase were harvested with the liquid phase by pipetting. The mass of dry residues was measured upon evaporation by boiling of the organic phase solvent.

### Results

Results identify that approximately 1.1 g of exudate mass was obtained from cultured plaque produced on LITSA media, for which exudate mass was statistically greater than for other media treatments. Mass produced on LITSA media was greater than for WBA media, which is a surprising result because plaque mass was greater for WBA than for LITSA media (Table 4, experiment 1). Comparison of exudate mass with total plaque identifies that refinement of plaque produced on LITSA media, the dry mass of harvested exudates comprised approximately 8.6 percent of the mass of cultured plaque. Concentration of MBAS in exudates from cultured plaque produced on LITSA media measured approximately 153 mg per g, which marked an enrichment of approximately 330-fold compared with the MBAS concentration of 464 μg per g in crude, cultured plaque produced on LITSA media at pH 7.0 (or parts per million; ppm), which is documented in Table 4.

Dry residues obtained from the solvent extraction measured 6.1 mg from approximately 1.00 g of cultured plaque. Concentration of MBAS in dry residues is not reported because residues were dried by evaporation at approximately 100 degrees C., which is known to cause protein denaturation and loss of bioactivity. However, surfactant as MBAS was measured in plaque at approximately 500 μg/g in similar plaque samples (Table 9) and the molar mass of protein CAB15086.1 (SEQ ID NO: 2) is 19.3 kDa compared with 288 kDa for SDS. (Said proteins refined by HPLC were estimated to be at least 1.36-fold more potent than SDS for MBAS per mol.) Therefore, total yield by mass of MBAS-inducing non-polar residues is projected at 24.6 mg per g of plaque, so recovery of non-polar residues as approximately 0.6 percent of plaque by mass represents up to 24.8 percent recovery.

Refinement procedures comprised of dilution, centrifugation, filtration and drying or solvent extraction and drying therefore yielded refined residues that are enriched or are projected to be enriched for methylene blue active substances comprising surfactant bioactivity. Refined plaque is projected to have increased utility as an animal feed additive where surfactant bioactivity is administered to an animal in the absence of cells or spores of strain 6A-1.

### Example 21

### Identification of Strain 6A-1 by Unique DNA Sequence.

The objective of the present example is to demonstrate that a unique DNA sequence SEQ ID NO: 1, referred to as “6360-1” is suitable for the use of identifying B. subtilis 6A-1 from other strains if B. subtilis. Additional genetic markers also are presented, such that B. subtilis 6A-1 is identified by testing positive for sequence 6360-1, negative for polyketide synthase PksJ (WP_003245563.1; SEQ IDNO: 10), and positive for vegetative catalase (NP_388762.2)) (SEQ ID NO: 12).

### Bacterial Strains

Five available strains of B. subtilis were obtained for study. Two strains denoted as BC-1 and BC-2 were cultured from a commercially available mixture (OPTI-BIOME®, BIO-CAT, Troy, Va. 22974). Strain E-1 was obtained from a commercial supplier (Envera, West Chester, Pa. 19380). Strain 168 is the common, research strain of Bacillus subtilis and was obtained from the American Type Culture Collection (ATCC 23857). Strain 6A1 is the strain maintained by Agri-King, Inc. (Fulton, Ill.). Each of the five strains was cultured aerobically in brain-heart-infusion medium at 35° C. for approximately 18 h. Bacterial cells were harvested by centrifugation at 3,000×g for 30 min.

### DNA Sequencing and Sequence Analysis

A total nucleic acids preparation was obtained from strain 6A-1 by using the Fast DNA-stool mini kit (Qiagen, Hilden, Germany). The DNA preparation was sequenced by shotgun methods (MiSeq, Illumina, San Diego, Calif. 92122). Sequence reads of strain 6A-1 were mapped to the known genome sequence of strain 168 (NCBI Reference Sequence NC_00964.3). The genome sequence of strain 6A-1 was aligned using BLAST software (Altschul, S. F., Gish, W. Miller, W. Myers, E. W. and Lipman, D. J. “Basic local alignment search tool.” J. Mol. Biol. 215:403-410 (1990)) to the genome sequence of strain 168 to discover regions of sequence dissimilarity. This analysis identified that, among numerous differences, genes encoding RefSeq proteins WP_003245563.1 (non-ribosomal peptide synthase), NP_389600.3 (polyketide synthase), were encoded in the genome of strain 168, but not strain 6A-1, whereas the gene encoding protein NP_388762.2 (vegetative catalase) was common to both strains.

Raw sequence reads obtained from strain 6A1 also were used to construct de novo assembly scaffolds via the Velvet assembler (Zerbino, D. R. and Birney, E. “Velvet: algorithms for de novo short read assembly using de Bruijn graphs.” Genome Res. 18.821-829 (2008)). De novo assembly yielded 1,513 genomic scaffolds. Each scaffold was analyzed for the presence of complete open reading frames, which encode for a biological protein sequence, and then open reading frames were aligned to genome sequence NC_00964.3 of strain 168 to detect genomic dissimilarity with strain 168. Open reading frames found to be prevalent in strain 6A-1 but not in strain 168 were queried against the NCBI non-redundant protein (nr) and non-redundant nucleotide (nr/nt) databases by using BLASTX and BLASTN, respectively (Carnacho C., Coulouris G., Avagyan V., Ma N., Papadopoulos J., Bealer K., and Madden T. L. “BLAST+: architecture and applications.” BMC Bioinformatics 10:421 (2008)) to hypothesize the presence of novel or unique genes present in the genome of strain 6A-1. An open reading frame accessioned as “sequence 6360-1” was found to have no significant sequence similarity to any sequence in the nr and nr/nt databases (Wheeler, David L., Barret, T. Benson, D Bryan, S., Canese, K. Chetvernin, V. Church, D. DiCuccio, M. Edgar, R. Federhen, S. Feolo, M. Geer, L., Helmberg, W. Kapustin, Y., Khovayko, O. Landsman, D., Lipman, D. Madden, T, Maglott, D. Miller, V. Ostell, i, Pruitt, K. Schuler, G. Shumway, M, Sequeira, E. Sherry, S. Sirotkin, K. Souvorov, A. Starchenko, G. Tatusov, R. Tatusova, T. Wagner, L. and Yaschenko, E. “Database resources of the national center for biotechnology information.” Nucleic Acids Res. 36:D13-D21(2008)). Sequence 6360-1 as a differentiating feature between strain 168 and strain 6A-1 is presented in Table 19.

### DNA Extraction and Quantitative PCR

Separately, total nucleic acids were extracted from strains BC-1, BC-2, E-1, 6A-1, or 168 by using the Fast DNA-stool mini kit (Qiagen, Hilden, Germany). Nucleic acids were eluted into water and DNA was quantified by Qubit fluorometric assay (Thermo-Fisher Scientific, Waltham, Mass. 02451). Preparations were diluted with water to approximately 1.0 ng/uL and assayed by quantitative PCR for the presence of genes encoding proteins WP_003245563.1 and NP_388762.2, or for sequence 6360-1. Quantitative PCR reactions were prepared by including 5 uL template DNA with 10 uL PrimeTime® Gene Expression Master Mix, and 1 uL each of forward primer, reverse primer, and fluorescent probe. Reactions were brought to final volume of 20 uL by addition of 2 uL water. Concentrations of forward and reverse primers in qPCR reactions were 900 nM each and concentration of probe in reactions was 250 nM. Sequences of primers and probes for each reaction are listed as footnotes in Table 20, which describes the detection of differentiating DNA sequences for the five strains.

### Results

Hypotheses were presented for the presence of differentiating genes between strain 6A-1 and strain 168 and three of these were tested by qPCR procedures. Quantitative PCR results confirmed the hypothesized presence or absence of respective genes in the two strains. Quantitative PCR results demonstrated that these differentiating genes were also useful for the differentiation of strain 6A-11 from strains BC-1, BC-2, and E-1. Importantly, only strain 6A-1 was positive for sequence 6360-1, which is not documented elsewhere in DNA sequence database nr/nt. Therefore, sequence 6360-1 is presented as novel DNA sequence that is suitable for the detection of strain 6A-1, and strain 6A-1 is fully differentiated from other strains of B. subtilis on the basis of sequence 6360-1 and genes encoding non-ribosomal peptide synthase, polyketide synthase, and vegetative catalase.

### Example 22

Recovery of B. subtilis 6A-1 DNA in Plaque Harvested from Solid Media Culture

The objective of the present example is to demonstrate that B. subtilis 6A-1 DNA was recovered from plaque of the same strain when said strain was cultured on solid media and harvested by scraping. The utility of the present example is to demonstrate that the bacterial plaque of 6A-1, which harbors polysaccharide degrading and surfactant fractions and can be utilized directly as an animal feed ingredient, can be identified as a culture product of the host strain. In the present example, DNA encoding sequence 6360-1 was detected by polymerase chain reaction to confirm the presence of DNA from strain 6A-1.

### Materials and Methods

Strain 6A-1 was cultured aerobically at 35 degrees C. for 24 hours on solid phase LITSA media at pH of 7.0. Biofilm plaque was obtained from plates by scraping and stored frozen. DNA was extracted from the biofilm plaque (approximately 300 mg) by using a commercial kit (Fast DNA Stool Mini Kit; Qiagen, Hilden, Germany). DNA was assayed by PCR for detection of sequence 6360-1 by using procedures described in Example 21. Approximately 10 ng of total DNA was utilized as template DNA.

### Results

Sequence 6360-1 was detected by PCR in triplicate at an amplification cycle count (mean±SEM) of 12.76±0.06 cycles. Detection of sequence 6360-1 in cultured plaque of strain 6A-1 demonstrates that this assay is of utility for detecting the presence of DNA from said strain in said composition that has utility as an animal feed additive.

### Example 23

Recovery of DNA of Strain 6A-1 in Cell-Free Substances Refined from Plaque of the Same Strain.

The objective of the present example is to demonstrate that DNA from strain 6A-1 is recoverable in exudates of said strain that are refined from cultured plaque to be free of cells and spores. The utility of the present example is a means of identifying that said exudates were produced by culturing said strain, where exudates are comprised of proteins CAB15086.1 (SEQ IDNO: 2) or CAB15055.1 (SEQ IDNO: 3) or CAB15943.1 (SEQ IDNO: 4) or a combination thereof. DNA in total cell-free substances is presumed to originate during the culture process from naturally ruptured or lysed bacterial cells of said strain.

### Materials and Methods

Strain 6A-1 was cultured aerobically for 24 hours at 35 degrees C. on solid phase TSA media at pH of 7.0. Cultures were established at commercial scale in metal pan and lid assemblies. Total plaque was obtained by scraping upon completion of culture. Exudates were refined from plaque by dilution, centrifugation, filtration, and lyophilization procedures described in example 20, and refined exudates were the same exudates presented in example 20, where exudates were refined from plaque cultured on TSA media.

Total DNA was extracted from refined plaque by reconstituting said plaque in water at 10 mg/mL. The dilution was subject to DNA extraction by a commercial kit (Fast DNA Stool Mini Kit; Qiagen, Hilden, Germany). DNA was assayed by PCR for detection of sequence 6360-1 by using procedures described in Example 20. Input template DNA measured <100 pg.

### Results

Sequence 6360-1 was detected by PCR in triplicate at an amplification cycle count (mean±SEM) of 21.66±0.15 cycles. Detection of sequence 6360-1 in cell-free substances refined from plaque of strain 6A-1 demonstrates that this assay is of utility for identifying strain 6A-1 as the source of a composition comprising exudates of the same strain, which has utility as an animal feed additive.

### TABLES

TABLE 1
Contaminant colony forming units³ per plate observed on different
solid phase media treated by autoclaving and incubated for 12, 24,
48, or 72 hours.

| | | Autoclave | | | | |
|---|---|---|---|---|---|---|
| Culture media | Measure | time, min | 12 h | 24 h | 48 h | 72 h |
| Experiment 1¹ | | | | | | |
| TSA | Total contaminants | 15 | NA | 0 | 0 | 1.5 |
| WBA, 3 percent | Total contaminants | 15 | NA | 0 | 0 | 0 |
| WBA, 6 percent | Total contaminants | 15 | NA | 0.5 | 2.5 | TNC |
| WBA, 9 percent | Total contaminants | 15 | NA | 5.5 | 26.5 | TNC |
| Experiment 2¹ | | | | | | |
| WBA, 9 percent | Total contaminants | 30 | 0 | 0 | NA | 1.5 |
| WBA, 9 percent | Total contaminants | 45 | 0 | 0 | NA | 0 |
| WBA, 9 percent | Total contaminants | 50 | 0 | 0 | NA | 2.5 |
| Experiment 3² | | | | | | |
| LITSA | Total coliform | 25 | NA | ND | NA | NA |
| | bacteria | | | | | |
| LITSA | Salmonellae | 25 | NA | ND | NA | NA |
| LITSA | Listeria spp. | 25 | NA | ND | NA | NA |
| LITSA | Staphylococcus | 25 | NA | ND | NA | NA |
| | aureus | | | | | |
| LITSA | Total mold spp. | 25 | NA | ND | NA | NA |
| LITSA | Total yeast spp. | 25 | NA | ND | NA | NA |
| LITSA | Clostridium | 25 | NA | ND | NA | NA |
| | perfringens | | | | | |

¹Value is the average of duplicate plates for each tested media and autoclave time.
²Value is the average of triplicate pan and lid assembly cultures, colony forming units per g.
³NA, not analyzed;
TNTC, too numerous to count;
ND, not detected

TABLE 2
Mass of plaque, g produced on a stack of three plates by different
media and oxygenation conditions.

| Media | Aerobic | Microaerophilic | P-value oxygen¹ |
|---|---|---|---|
| MBM | 0.08 ± 0.02^{c} | 0.10 ± 0.03^{c} | 0.714 |
| TSA | 0.56 ± 0.13^{b} | 0.51 ± 0.06^{b} | 0.718 |
| WBM | 1.05 ± 0.20^{a} | 1.18 ± 0.08^{a} | 0.510 |
| P-value | 0.008 | <0.001 | |
| media | | | |

¹P-value is for the comparison of means within a row by two-sample t-test.
^{a,b,c}Means in a column that do not share a common superscript are different, P < 0.1.

TABLE 3
Biomass and colony forming units produced by liquid phase or
solid phase culture of strain 6A-1.

| | Liquid Phase | Solid Phase | P-value |
|---|---|---|---|
| Total CFU, log₁₀ | 10.05 ± 0.07 | 9.72 ± 0.12 | 0.031 |
| Total mass, mg | 52.6 ± 11.0 | 30.0 ± 2.0 | 0.104 |
| Mass per CFU, pg | 5.05 ± 1.70 | 5.86 ± 0.93 | 0.696 |

¹P-value is for the comparison of means within a row by two-sample t-test.
^{a,b,c}Means in a column that do not share a common superscript are different, P < 0.10.

TABLE 4
Mass of cultured plaque produced by aerobic solid phase culture on different types of media.

| | | | | | | |
|---|---|---|---|---|---|---|
| Basal | | | n per | Surface | Time, | |
| media | pH | Enrichment | treatment | area, cm² | h | Mass, g |

Experiment 1⁴

| | | | | | | |
|---|---|---|---|---|---|---|
| MBM | 7.0 | — | 2 | 1,277 | 24 | 1.83 ± 0.06^{c} |
| TSA | 7.0 | — | 2 | 1,277 | 24 | 10.27 ± 2.42^{b} |
| LITSA | 7.0 | — | 2 | 1,277 | 24 | 13.21 ± 0.46^{ab} |
| WBA | 7.0 | — | 2 | 1,277 | 24 | 20.08 ± 0.80^{a} |

Experiment 2^{1, 4}

| | | | | | | |
|---|---|---|---|---|---|---|
| MBM | 7.0 | — | 3 | 235 | 24 | 0.08 ± 0.02^{c} |
| TSA | 7.0 | — | 3 | 235 | 24 | 0.56 ± 0.13^{b} |
| LITSA | 7.0 | — | 3 | 235 | 24 | 0.91 ± 0.03^{ab} |
| WBA | 7.0 | — | 3 | 235 | 24 | 1.05 ± 0.20^{a} |

Experiment 3²

| | | | | | | |
|---|---|---|---|---|---|---|
| TSA | 4.5 | — | 1 | 785 | 24 | NA³ |
| TSA | 5.0 | — | 1 | 785 | 24 | NA³ |
| TSA | 5.5 | — | 1 | 785 | 24 | 2.17 |
| TSA | 6.0 | — | 1 | 785 | 24 | 2.26 |
| TSA | 6.5 | — | 1 | 785 | 24 | 2.50 |
| TSA | 7.0 | — | 1 | 785 | 24 | 2.85 |
| TSA | 7.5 | — | 1 | 785 | 24 | 1.96 |

Experiment 4⁴

| | | | | | | |
|---|---|---|---|---|---|---|
| LITSA | 6.0 | — | 3 | 235 | 24 | 0.67 ± 40^{a} |
| LITSA | 7.0 | — | 3 | 235 | 24 | 0.67 ± 160^{a} |
| LITSA | 8.0 | — | 3 | 235 | 24 | 0.84 ± 20^{a} |
| LITSA | 9.0 | — | 3 | 235 | 24 | 0.22 ± 20^{b} |
| LITSA | 10.0 | — | 3 | 235 | 24 | NA⁵ |

Experiment 5

| | | | | | | |
|---|---|---|---|---|---|---|
| WBA | 6.0 | — | 3 | 235 | 24 | 1.60 ± 0.03^{c} |
| WBA | 7.0 | — | 3 | 235 | 24 | 2.52 ± 0.04^{a} |
| WBA | 8.0 | — | 3 | 235 | 24 | 2.08 ± 0.08^{b} |
| WBA | 9.0 | — | 3 | 235 | 24 | 0.03 ± 0.01^{d} |
| WBA | 10.0 | — | 3 | 235 | 24 | NA⁵ |

Experiment 6⁶

| | | | | | | |
|---|---|---|---|---|---|---|
| LITSA | 8.0 | — | 3 | 235 | 24 | 0.83 ± 0.06^{b} |
| LITSA | 8.0 | Iron proteinate, | 3 | 235 | 24 | 1.29 ± 0.05^{a} |
| | | 0.33 g/L | | | | |
| LITSA | 8.0 | Iron proteinate, | 3 | 235 | 24 | 1.25 ± 0.03^{a} |
| | | 0.66 g/L | | | | |
| LITSA | 8.0 | Iron proteinate, | 3 | 235 | 24 | 1.35 ± 0.03^{a} |
| | | 1.33 g/L | | | | |

Experiment 7⁶

| | | | | | | |
|---|---|---|---|---|---|---|
| LITSA | 8.0 | — | 3 | 1,277 | 22 | 8.50 ± 1.00^{a} |
| LITSA | 8.0 | Iron proteinate, | 3 | 1,277 | 22 | 9.39 ± 0.54^{a} |
| | | 1.33 g/L | | | | |

Experiment 8⁴

| | | | | | | |
|---|---|---|---|---|---|---|
| LITSA | 7.0 | — | 3 | 1,277 | 22 | 12.98 ± 0.75^{a} |
| LITSA | 7.0 | Calcium acetate, | 3 | 1,277 | 22 | 7.62 ± 0.39^{b} |
| | | 125 mM | | | | |
| LITSA | 7.0 | Calcium acetate, | 3 | 1,277 | 22 | 4.90 ± 0.45^{b} |
| | | 250 mM | | | | |
| LITSA | 7.0 | Sodium acetate, | 3 | 1,277 | 22 | 6.90 ± 2.20^{b} |
| | | 250 mM | | | | |
| LITSA | 7.0 | Sodium acetate, | 3 | 1,277 | 22 | 4.57 ± 0.71^{b} |
| | | 500 mM | | | | |

Experiment 9⁴

| | | | | | | |
|---|---|---|---|---|---|---|
| LITSA | 7.0 | — | 3 | 1,277 | 22 | 11.50 ± 0.45^{a} |
| LITSA | 7.0 | Calcium chloride, | 3 | 1,277 | 22 | 7.18 ± 0.37^{b} |
| | | 100 mM | | | | |
| LITSA | 7.0 | Calcium nitrate, | 3 | 1,277 | 22 | 7.25 ± 0.48^{b} |
| | | 100 mM | | | | |

Experiment 10

| | | | | | | |
|---|---|---|---|---|---|---|
| LITSA | 7.0 | — | 3 | 1,277 | 22 | 16.29 ± 0.74^{a} |
| LITSA | 7.0 | Calcium chloride, | 3 | 1,277 | 22 | 5.28 ± 0.16^{b} |
| | | 30 mM | | | | |
| LITSA | 7.0 | Calcium chloride, | 3 | 1,277 | 22 | 6.12 ± 0.37^{b} |
| | | 60 mM | | | | |

Experiment 11⁴

| | | | | | | |
|---|---|---|---|---|---|---|
| LITSA | 7.0 | — | 2 | 1,277 | 22 | 20.10 ± 0.10^{a} |
| LITSA | 7.0 | Sodium chloride, | 2 | 1,277 | 22 | 16.79 ± 1.9^{a} |
| | | 60 mM | | | | |
| LITSA | 8.0 | — | 2 | 1,277 | 22 | 9.27 ± 0.26^{b} |

¹Partial data are duplicated from example 3.
²Cultures were established as a single stack of 10 Petri plates for each media treatment.
³Not analyzed, media did not set properly as solid phase.
⁴Data were analyzed by ANOVA and treatments means were separated by Tukey HSD test.
⁵Not analyzed, insufficient sample for analysis.
⁶Data were analyzed by two-sample t-test.
^{a, b, c}Treatment means within an experiment that do not share a common superscript are different, P < 0.05.

TABLE 5
Mass of 6A-1 plaque produced by solid phase culture with
supplemental calcium acetate or sodium acetate at dissolved
anion concentration of 250 mM or 500 mM.

| | Supplemental concentration of acetate | | | |
|---|---|---|---|---|
| Media | 0 mM | 250 mM | 500 mM | P-value |
| LITSA + | 12.98 ± 0.75^{a} | 7.62 ± 0.39^{b} | 4.90 ± 0.45^{c} | <0.001 |
| Calcium acetate | | | | |
| LITSA + | | 6.90 ± 2.20 | 4.57 ± 0.71 | |
| Sodium acetate | | | | |
| P-value, two- | | 0.726 | 0.712 | |
| sample t-test | | | | |

^{a,b,c}Means in the same row that do not share a common superscript are different, P < 0.05.

TABLE 6
Concentration of cellulase bioactivity in plaque of strain 6A-1 produced by different
culture conditions.

| | | | | | | | |
|---|---|---|---|---|---|---|---|
| | | | | | | | Cellulase |
| | | | | Preparative | Surface | | activity, |
| Basal | | | n per | dilution | area, | Time, | DNS units |
| media | pH | Oxygenation | treatment | (w/v) | cm² | h | per g |

Experiment 1

| | | | | | | | |
|---|---|---|---|---|---|---|---|
| MBM | 7.0 | Aerobic | 3 | 500 | 235 | 24 | 1.72 ± 0.61^{b} |
| MBM | 7.0 | Microaerophilic | 3 | 500 | 235 | 24 | 0.66 ± 0.13^{b} |
| TSA | 7.0 | Aerobic | 3 | 500 | 235 | 24 | 0.30 ± 0.21^{b} |
| TSA | 7.0 | Microaerophilic | 3 | 500 | 235 | 24 | 0.49 ± 0.39^{b} |
| LITSA | 7.0 | Aerobic | 3 | 500 | 235 | 24 | 1.88 ± 1.35^{b} |
| WBA | 7.0 | Aerobic | 3 | 500 | 235 | 24 | 19.00 ± 2.91^{a} |
| WBA | 7.0 | Microaerophilic | 3 | 500 | 235 | 24 | 21.70 ± 1.70^{a} |

Experiment 2

| | | | | | | | |
|---|---|---|---|---|---|---|---|
| WBA | 6.0 | Aerobic | 3 | 10 | 235 | 24 | 0.38 ± 0.04^{b} |
| WBA | 7.0 | Aerobic | 3 | 10 | 235 | 24 | 0.58 ± 0.03^{a} |
| WBA | 8.0 | Aerobic | 3 | 10 | 235 | 24 | 0.55 ± 0.02^{a} |
| WBA | 9.0 | Aerobic | 3 | 10 | 235 | 24 | NA |
| WBA | 10.0 | Aerobic | 3 | 10 | 235 | 24 | NA |

^{a, b, c}Treatment means within an experiment that do not share a common superscript are different, P < 0.05, Tukey HSD.

TABLE 7
Identification by liquid chromatography and mass spectrometry
of proteins separated by two-dimensional gel electrophoresis.

| | | | | Estimated |
|---|---|---|---|---|
| Band | | | Estimated | mass, |
| Number | Protein ID¹ | Protein description | pI | kDa |
| 1, 2 | CAB13343.1 | Protease NprE | 7.16 | 56.5 |
| | (SEQ ID NO: 5) | | | |
| 3, 4, | CAB12870.2 | Protease subtilisin E; | 9.04 | 39.5 |
| 5, 6 | (SEQ ID NO: 6) | protease aprE | | |
| 7 | CAB15943.1 | Endo-beta-1,3-1,4 | 6.41 | 27.3 |
| | (SEQ ID NO: 4) | glucanase | | |
| 8, 9 | CAB13625.2 | Sporulation-specific | 5.95 | 27.1 |
| | (SEQ ID NO: 7) | N-acetylmuramoyl- | | |
| | | L-alanine amidase | | |
| 10 | CAB13776.1 | endo-1, 4-beta- | 9.44 | 23.3 |
| | (SEQ ID NO: 8) | xylanase | | |
| 11 | CAB11814.1 | Fragment, methionyl- | 5.14 | 76.2 |
| | (SEQ ID NO: 9) | tRNA synthetase | | |

¹GenBank accession number

TABLE 8
Surface tension and concentration of methylene blue active
substances of solutions comprised of aqueous or non-polar
solvent-extracted residues from cultured plaque of
strain 6A-1.

| | | Methylene blue |
|---|---|---|
| | | active substances, |
| | Surface tension, | SDS equivalents, |
| | dynes/cm | μg/mL¹ |
| Aqueous residues | 66.2 | ND |
| Non-polar residues | 57.6 | 2.68 |
| Tris-Cl buffer | 68.6 | ND |
| Water | 67.4 | ND |

¹Limit of detection, or LOD = 0.25 μg/mL; ND, not detected.

TABLE 9
Concentration of surfactant bioactivity quantified as MBAS1 in plaque of strain 6A-1
produced by different culture conditions.

| | | | | | | | | |
|---|---|---|---|---|---|---|---|---|
| | | | | | Preparative | Surface | | |
| Basal | | | | n per | dilution | area, | Time, | MBAS, |
| media | pH | Oxygenation | Enrichment | treatment | (w/v) | cm² | h | μg/g |

Experiment 1

| | | | | | | | | |
|---|---|---|---|---|---|---|---|---|
| MBM | 7.0 | Aerobic | — | 3 | 500 | 235 | 24 | 63 ± |
| | | | | | | | | 15^{c} |
| TSA | 7.0 | Aerobic | — | 3 | 500 | 235 | 24 | 332 ± |
| | | | | | | | | 25^{a} |
| LITSA | 7.0 | Aerobic- | — | 3 | 500 | 235 | 24 | 228 ± |
| | | | | | | | | 88^{ab} |
| WBA | 7.0 | Aerobic | — | 3 | 500 | 235 | 24 | 112 ± |
| | | | | | | | | 25^{bc} |

Experiment 2

| | | | | | | | | |
|---|---|---|---|---|---|---|---|---|
| MBM | 7.0 | Microaerophilic | — | 4 | 500 | 235 | 24 | 119 ± |
| | | | | | | | | 48^{b} |
| TSA | 7.0 | Microaerophilic | — | 4 | 500 | 235 | 24 | 406 ± |
| | | | | | | | | 25^{a} |
| WBA | 7.0 | Microaerophilic | — | 4 | 500 | 235 | 24 | 55 ± |
| | | | | | | | | 6^{b} |

Experiment 3

| | | | | | | | | |
|---|---|---|---|---|---|---|---|---|
| LITSA | 6.0 | Aerobic | — | 3 | 200 | 235 | 24 | 517 ± |
| | | | | | | | | 79^{a} |
| LITSA | 7.0 | Aerobic | — | 3 | 200 | 235 | 24 | 371 ± |
| | | | | | | | | 102^{a} |
| LITSA | 8.0 | Aerobic | — | 3 | 200 | 235 | 24 | 413 ± |
| | | | | | | | | 25^{a} |
| LITSA | 9.0 | Aerobic | — | 3 | 200 | 235 | 24 | 6.5 ± |
| | | | | | | | | 4.9^{b} |
| LITSA | 10.0 | Aerobic | — | 3 | 200 | 235 | 24 | NA² |

Experiment 4

| | | | | | | | | |
|---|---|---|---|---|---|---|---|---|
| LITSA | 7.0 | Aerobic | — | 3 | 350 | 1,277 | 22 | 464 ± |
| | | | | | | | | 18^{b} |
| LITSA | 7.0 | Aerobic | Calcium | 3 | 350 | 1,277 | 22 | 900 ± |
| | | | chloride, | | | | | 19^{a} |
| | | | 100 mM | | | | | |
| LITSA | 7.0 | Aerobic | Calcium | 3 | 350 | 1,277 | 22 | 855 ± |
| | | | nitrate, | | | | | 34^{a} |
| | | | 100 mM | | | | | |

Experiment 5

| | | | | | | | | |
|---|---|---|---|---|---|---|---|---|
| LITSA | 8.0 | Aerobic | — | 3 | 350 | 1,277 | 22 | 525 ± |
| | | | | | | | | 58^{a} |
| LITSA | 8.0 | Aerobic | Iron | 3 | 350 | 1,277 | 22 | 489 ± |
| | | | proteinate, | | | | | 33^{a} |
| | | | 1.33 g/L | | | | | |

¹MBAS, methylene blue active substances.
²Not analyzed, insufficient plaque for analysis.
^{a, b, c}Treatment means within an experiment that do not share a common superscript are different, P < 0.05, Tukey HSD or two sample t-test.

TABLE 10
Concentration of MBAS in cultured plaque of
Bacillus subtilis strains 6A-1, 168, and PB6.

| Strain | MBAS, μg/g |
|---|---|
| Bacillus subtilis 6A-1 | 164.5 ± 10.0^{a} |
| Bacillus subtilis 168 | 19.3 ± 0.5^{b} |
| Bacillus subtilis PB6 | 41.5 ± 0.3^{b} |

^{a,b}Means without a common superscript are different, P < 0.05.

TABLE 11
Measurements (mean ± SEM) of calcium absorption and
excretion after 17 d as control or treatment.

| | | | |
|---|---|---|---|
| | | Paired | |
| | | difference | |
| Control | Treatment | (Trt - Con) | P-value |

Experiment 1¹

| | | | | |
|---|---|---|---|---|
| Excreted Ca, mg | 229 ± 41 | 168 ± 22 | −60.8 ± 34.5 | 0.106 |
| Apparent absorbed Ca, g | 11.6 ± 1.1 | 14.4 ± 1.6 | 2.82 ± 2.00 | 0.185 |
| Excreted per absorbed | 20.7 ± 3.4 | 11.6 ± 1.0 | −9.09 ± 3.59 | 0.028 |
| (mg:g) | | | | |

Experiment 2²

| | | | | |
|---|---|---|---|---|
| Excreted Ca, mg | 176 ± 34 | 147 ± 25 | −29.5 ± 25.2 | 0.268 |
| Apparent absorbed Ca, g | 13.3 ± 1.8 | 15.2 ± 1.4 | 1.97 ± 2.16 | 0.382 |
| Excreted per absorbed | 14.9 ± 2.5 | 9.3 ± 1.3 | −5.56 + 2.27 | 0.034 |
| (mg:g) | | | | |

¹Sheep were fed a diet comprised of approximately 3.0 percent oil.
²Sheep were fed a diet comprised of approximately 5.0 percent oil.

TABLE 12
Concentration of calcium in dry liver tissue from sheep that were
supplemented or not supplemented with spores of strain 6A-1.

| | Spore | Dietary | | | |
|---|---|---|---|---|---|
| | dose, | Ca, | | | |
| | CFU/lb.¹ | percent¹ | Control | Treatment | P-value |
| Experiment | 1.4 × 10¹⁰ | 0.99 | 157 ± 12 | 200 ± 7 | 0.007 |
| 1 | | | | | |
| Experiment | 1.1 × 10¹⁰ | 1.16 | 240 ± 11 | 262 ± 14 | 0.214 |
| 2 | | | | | |

¹On a dry matter basis in feed.

TABLE 13
Concentration of calcium in fecal dry matter
after 28 days of supplementation.

| | Control | Treatment | P-value |
|---|---|---|---|
| Calcium, mg/kg | 3.24 ± 0.18 | 2.72 ± 0.22 | 0.095 |

TABLE 14
Calcium concentration in liver tissue.

| | Ca, μg/g | Log₁₀ Ca, μg/g |
|---|---|---|
| Treatment, | | |
| mean ± SEM | | |
| Control | 105 ± 24 | 1.89 ± 0.13^{a} |
| RP10 | 226 ± 63 | 2.15 ± 0.17^{a} |
| RP25 | 165 ± 37 | 2.09 ± 0.11^{a} |
| RP50 | 246 ± 32 | 2.33 ± 0.08^{a} |
| RP100 | 162 ± 29 | 2.06 ± 0.16^{a} |
| P-value | NA | 0.250 |
| Aggregate by biomass | | |
| supplementation, | | |
| mean ± SEM | | |
| Control | 105 ± 24 | 1.89 ± 0.13^{a} |
| RP-aggregate | 198 ± 20 | 2.16 ± 0.07^{b} |
| P-value | NA¹ | 0.097 |

¹NA, not analyzed.
^{a,b}Means with different superscripts are different, P < 0.10.

TABLE 15
Measurements of calcium digestibility, urinary excretion,
and retention in pigs not supplemented or supplemented
with spores of strain 6A-1.

| | Control | 6A-1 | P-value |
|---|---|---|---|
| Apparent total tract | 56.4 ± 2.3 | 56.9 ± 3.1 | 0.908 |
| digestibility, percent | | | |
| Urinary excretion | 5.79 ± 0.36 | 5.09 ± 0.53 | 0.273 |
| (log₁₀[Ca, ppm]: | | | |
| Creatinine, g/dL) | | | |
| Excretion: digestibility | 122 ± 12^{a} | 86 ± 8^{b} | 0.029 |

^{a,b}Means in the same row with different superscripts are different, P < 0.05.

TABLE 16
Titrated effect of orally delivered 6A-1 spores on dairy cow milk fatty acid
composition as percent of total fatty acids.

| | 0.0 × 10⁰ | 1.0 × 10⁶ | 1.0 × 10⁷ | 1.0 × 10⁸ | 1.0 × 10⁹ | P-value¹ |
|---|---|---|---|---|---|---|
| Saturated | 69.8 ± 1.9 | 70.2 ± 1.4 | 67.5 ± 1.4 | 65.8 ± 1.4 | 66.3 ± 2.2 | 0.029 |
| fatty acids | | | | | | |
| Palmitic | 32.0 ± 1.6 | 33.0 ± 1.0 | 29.9 ± 0.5 | 30.3 ± 1.0 | 30.8 ± 1.0 | 0.147 |
| acid | | | | | | |
| (16:0) | | | | | | |
| Unsaturated | 30.2 ± 1.9 | 29.8 ± 1.4 | 32.5 ± 1.4 | 34.2 ± 1.4 | 33.7 ± 2.2 | 0.029 |
| fatty acids | | | | | | |
| Mono- | | | | | | |
| unsaturated | 26.2 ± 1.9 | 26.1 ± 1.4 | 28.4 ± 1.3 | 30.1 ± 1.4 | 29.6 ± 2.0 | 0.030 |
| fatty acids | | | | | | |
| Oleic acid | 20.5 ± 1.8 | 20.1 ± 1.0 | 22.4 ± 1.0 | 24.2 ± 1.3 | 23.4 ± 1.5 | 0.025 |
| (18:1) | | | | | | |
| Poly- | 4.00 ± 0.09 | 3.78 ± 0.12 | 4.10 ± 0.17 | 4.11 ± 0.12 | 4.12 ± 0.15 | 0.173 |
| unsaturated | | | | | | |
| fatty acids | | | | | | |
| Linoleic | 2.24 ± 0.07 | 2.19 ± 0.10 | 2.57 ± 0.14 | 2.52 ± 0.07 | 2.52 ± 0.17 | 0.053 |
| acid | | | | | | |
| (18:2) | | | | | | |

¹Statistical P-value describes the linear effect of incrementally increased spore dose

TABLE 17
Fatty acid methyl ester analysis of ribeye and fat tissue in
beef feeder animals.

| | Control | 6A-1 | P-value |
|---|---|---|---|
| Ribeye tissue | | | |
| Palmitic acid, C16:0 | 28.3 ± 0.3 | 25.8 ± 0.8 | 0.011 |
| Stearic acid, C18:0 | 13.4 ± 0.9 | 14.7 ± 0.7 | 0.284 |
| Oleic acid, c9-C18:1 | 33.4 ± 0.6 | 33.1 ± 0.4 | 0.754 |
| Elaidic acid, t9-C18:1 | 6.5 ± 0.6 | 8.0 ± 0.5 | 0.081 |
| Other | 18.4 ± 0.7 | 18.5 ± 1.0 | 0.969 |
| Fat tissue | | | |
| Palmitic acid, C16:0 | 26.6 ± 0.3 | 25.3 ± 0.3 | 0.010 |
| Stearic acid, C18:0 | 9.7 ± 0.6 | 9.6 ± 0.6 | 0.871 |
| Oleic acid, c9-C18:1 | 37.2 ± 0.8 | 36.5 ± 0.8 | 0.536 |
| Elaidic acid, t9-C18:1 | 7.5 ± 1.0 | 8.8 ± 0.6 | 0.282 |
| Other | 18.8 ± 0.7 | 19.8 ± 0.9 | 0.423 |

TABLE 18
Mass of exudates obtained from cultured plaque
of 6A-1 produced¹ on different media.

| | | | |
|---|---|---|---|
| | Extraction | | |
| Media | method | Mass, mg | MBAS, mg/g |

Refinement set 1

| | | | |
|---|---|---|---|
| MBM | Total exudates | 73 ± 20^{d} | 66.8 ± 1.8 |
| TSA | Total exudates | 417 ± 156^{c} | 157.1 ± 103.5 |
| LITSA | Total exudates | 1,140 ± 132^{a} | 153.5 ± 51.3 |
| WBA | Total exudates | 766 ± 24^{b} | 33.9 ± 6.3 |

Refinement set 2

| | | | |
|---|---|---|---|
| LITSA | Solvent-extracted | 6.1 | NA² |

¹Cultures were established on pan and lid assemblies in duplicate.
²NA, not analyzed
^{a,b,c,d}Means within the same refinement set that do not share a common superscript are different, P < 0.10.

TABLE 19
Hypothesized coordinates of genes and open reading frames in
genomic sequences of strain 168 and strain 6A1.

| | Strain 168; | |
|---|---|---|
| Gene Identifier | NC_00964.3 | Strain 6A-1 |
| WP_003245563.1 | NC00964.3: 1792806- | Absent |
| Polyketide synthase | 1807937 | |
| PksJ (SEQ ID NO: 10) | | |
| NP_389600.3 | NC:009643: 1807921- | Absent |
| Polyketide synthase | 1821534 | |
| PksL (SEQ ID NO: 11) | | |
| NP_388762.2 (SEQ ID | NC_00964.3: 959535- | AL009126.3: |
| NO: 12) | 960986 | 959487-960926 |
| Vegetative catalase 1, | | |
| katA | | |
| Sequence 6360-1 | Absent | Non-chromosomal |
| (SEQ ID NO: 1) | | scaffold 6360: 14- |
| | | 577 |

TABLE 20
Detection¹ by qPCR (cycle threshold) of
differentiating DNA sequence among
5 strains of Bacillus subtilis.

| | | | Sequence |
|---|---|---|---|
| Strain | WP_003245563.1² | NP_388762.2³ | 6360-1⁴ |
| BSBC-1 | 10.16 | 12.50 | ND |
| BSBC-2 | 11.94 | 13.15 | ND |
| BSE-1 | NEG | NEG | ND |
| BS168 | 11.42 | 12.90 | ND |
| BS6A1 | NEG | 13.11 | 12.35 |

¹ND, not detected.
²F: 5-GTACATATTGGCAGAAACAGCTATC-3 (SEQ ID NO: 13)
R: 5-CCTGGTGTATGTATCCTCTCTAAAC-3 (SEQ ID NO: 14)
P: 5-TGTCAGCGCAAGTTCAGTCGATTCT-3 (SEQ ID NO: 15)
³F: 5-CCGAAGCTGTTAGGCTCGTAAT-3 (SEQ ID NO: 16)
R: 5-CGCGCACGCAACAAAGTAAA-3 (SEQ ID NO: 17)
P: 5-CGCATTTGCCCATCACGCTGATAATT-3 (SEQ ID NO: 18)
⁴F: 5-TTTGGGTCTACATGGTGGATAAG (SEQ ID NO: 19)
R: 5-AACCCTAATCTTACTGGTCAGTTC (SEQ ID NO: 20)
P: 5-TGCAGGATCATAGCTGATCTCAAATCGC (SEQ ID NO: 21)

### REFERENCES

Altschul, S. F., Gish, W. Miller, W. Myers, E. W. and Lipman, D. J. “Basic local alignment search tool.” J. Mol. Biol. 215:403-410 (1990).
Altschul, J. Mol. Biol. 36: 290-300 (1993)
Anuradha, S. N. “Structural and Molecular Characteristics of Lichenysin and its Relationship to Surface Activity” In: Sen, R. (ed) Biosurfactants Advances in Experimental Medicine and Biology. Vol. 672 Springer, New York, N.Y. (2010).
[AOAC] Association of Official Analytical Chemists. “Metals and other elements in plants and pet foods: inductively coupled plasma spectroscopic method, AOAC official method 985.01.” (2003).
ASTM D2330-20, Standard Test Method for Methylene Blue Active Substances, ASTM International, West Conshohocken, Pa., 2020, astm.org.
Ayangbile, G., Grzemski, M., Tobey, J. F., Spangler, D., Krueger, L., U.S. patent Ser. No. 10/138,444. “Bacteria and enzymes produced therefrom and methods of using same.” (2018).
Ausubel et al., eds. (1995) Current Protocols in Molecular Biology, Chapter 2 (Greene Publishing and Wiley-Interscience, New York)
Batzer et al. (1991) Nucleic Acid Res. 19:5081
Carnacho C., Coulouris G., Avagyan V., Ma N., Papadopoulos J, Bealer K., and Madden T. L. “BLAST+: architecture and applications.” BMC Bioinformatics 10:421 (2008).
Cameotra, S. S., and Makker, R. S. “Recent Applications of Biosurfactant as Biological and Immunological Molecules,” Curr. Opin. Microbiol. 7:262-266 (2004).
Cheng, Y. H., Zhang, N., Han, J. C., Chang, C. W., Hsiao, F. S. H. and Yu, Y. H. “Optimization of surfactin production from Bacillus subtilis in fermentation and its effects on Clostridium perfringens-induced necrotic enteritis and growth performance in broilers.” J. Anim. Physiol. Anim. Nutr. 102(5):1232-1244 (2018).
Corpet (1988), Nucleic Acids Res. 16:10881-10890
Creighton, Proteins: Structures and Molecular Properties (WH Freeman and Co.; 2nd edition (December 1993)
Davila-Ramos, H., Gonzalez-Castellon, A., Barreras-Serrano, A., Estrada-Angulo, A., Lopez-Soto, M. A., Macias-Zamora, J. V., A Plascencia, A. Vega, S. H., and Zinn, R. A. “Influence of Method of Surfactant Supplementation on Characteristics of Digestion and Feeding Value of Fat in Holstein Steer Fed a High-Energy Finishing Diet.” J. Appl. Anim. Res. 39(3):192-195 (2011).
Davis et al., “Basic Methods in Molecular Biology” Appleton and Lange, Norwalk, Conn. (1994).
Demain, A. L. “Minimal media for quantitative studies with Bacillus subtilis.”. J. Bactriol. 75:517-522 (1958).
EFSA Panel on Additives and Products or Substances used in Animal Feed (FEEDAP). “Guidance on the assessment of the toxigenic potential of Bacillus species used in animal nutrition.” EFSA Journal 12(5):3665 (2014).
Gacs, G. and Barltrop, D. “Significance of Ca-soap formation for calcium absorption in the rat.” Gut 18:64-68 (1977).
(Geys, R., Soetaert, W., and Van Bogaert, I. “Biotechnological Opportunities in Biosurfactant Production,” Curr. Opin. Biotechnol. 30:66-72 (2014).
Green, A. J., Littlejohn, K. A., Hooley, P. and Cox, P. W. “Formation and stability of food foams and aerated emulsions: Hydrophobins as novel functional ingredients. Curr. Opin. Colloid. In. 18(4):292-301 (2013).
Gundina, E. J., Fernandes, E. C., Rodrigues, A. I., Teixeira, J. A., and Rodriques, L. R. “Biosurfactant Production by Bacillus subtilis Using Corn Steep Liquor as Culture Medium.” Front. Microbiol. 6:1-7 (2015).
Haymes et al. (1985) In: Nucleic Acid Hybridization, a Practical Approach, IRL Press, Washington, D.C.
Hein (1994), Methods Mol. Biol. 25:349-364.
Henikoff (1992), Proc. Nat. Acad. Sci. USA 89:10915-10919.
Henikoff and Henikoff (1993), Proteins 17: 49-61.
Higgins (1988), Gene 73: 237-244.
Ho, G. H., Ho, T. I., Hsieh, K. H., Su, Y. C., Lin, P. Y., Yang, J., Yang, K, H., and Yang, S. C. “Gamma-Polyglutamic Acid Produced by Bacillus subtilis (natto): Structural Characteristics, Chemical Properties and Biological Functionalities.” J. Chin. Chem. Soc. 53:1363-1384 (2006).
Hobley, L., Ostrowski, A., Rao, F. V., Bromley, K. M., Porter, M. Prescott, A. R., MacPhee, C.
E., Van Aalten, D. N. F., and Stanley-Wall, N. R. “BslA is a self-assembling bacterial hydrophobin that coats the Bacillus subtilis biofilm.” Proc. Nat. Acad. Sci. 110(33):13600-13605 (2013).
Horowitz, S., Gilbert, J. N., and Griffin, W. M. “Isolation and Characterization of a Surfactant Produced by Bacillus lichenformis 86.” J Ind. Microbiol. 6:243-248 (1990).
Hu, M., Li, Y. Decker, E. A., and McClements, D. J. “Role of calcium and calcium-binding agents on the lipase digestibility of emulsified lipids using an in vitro digestion model. Food Hydrocolloid, 24(8):719-725 (2010).
Huang, Computer Applications in the Biosciences 8:155-165 (1992).
Innis, M., Gelfand, D. and Sninsky, J. (1995) PCR Strategies. Academic Press, New York. Innis, M., Gelfand, D. and Sninsky, J. (1999) PCR Applications: Protocols for Functional Genomics, Academic Press, New York.
Jurado, E., Fernandez-Serrano, M., Nunez-Olea, J., Luzon, G., and Lechuga, M. “Simplified Spectrophotometric Methods using Methylene Blue for Determining Anionic Surfactants: Applications to the Study of Primary Biodegradation in Aerobic Screening Tests.” Chemosphere 65:278-285 (2006).
Karlin (1993), Proc. Nat. Acad. Sci. USA 90:5873-5787.
Kobayashi, K. and Megumi I. “BslA (YuaB) forms a hydrophobic layer on the surface of Bacillus subtilis biofilms.” Mol. Microbiol. 85(1):51-66 (2012).
Korsten, L., and Cook, N. “Optimizing culturing conditions for Bacillus subtilis.” South African Avocado Growers' Association Yearbook, 19:54-58 (1996).
Lee, S. S., Kim, H. S., Moon, Y. H., Choi, N. J. and Ha, J. K. “The effects of a non-ionic surfactant on the fermentation characteristics, microbial growth, enzyme activity and digestibility in the rumen of cows”. Anim. Feed Sci. Tech. 115:37-50 (2004).
Marvasi, M., Visscher, P. T., and Martinez, L. C. “Exopolymeric substances (EPS) from Bacillus subtilis: Polymers and genes encoding their synthesis.” FEMS Microbiol. Lett., 313:1-9 (2010).
McAllister, T. A., Stanford, K., Bae, H. D., Treacher, R. J., Hristov, A. N., Baah, J., Shelford, J. A., and Cheng, K-J. “Effect of a Surfactant and Exogenous Enzymes on Digestibility of Feed and on Growth Performance and Carcass Traits of Lambs,” Can. J. Anim. Sci. 80(1):35-44 (2000).
Meinkoth and Wahl, Anal. Biochem., 138:267-284 (1984).
Mitchaothai, J., Yuangklang, C., Vasupen, K., Wongsuthavas, S. and Beynen, A. C. “Effect of dietary calcium and lecithin on growth performance and small intestinal morphology of young wild pigs.” Livest Sci., 134:106-108 (2010).
Morris, R. J., Schor, M. Gillespie, R. M., Ferreira, A. S, Baldauf, L. Earl, C. Ostrowski, A. Hobley, L., Bromley, K M., Sukhodub, I. and Arnaouteli, S. “Natural variations in the bioilm-associated protein BslA from the genus Bacillus.” Sci. Rep. 7:1-13 (2017).
Morrison, (1997) Mol. Biol. Evol. 14:428-441.
Needleman and Wunsch (J. Mol. Biol. 48:443-453 (1970).
Neu, T. R. “Significance of Bacterial Surface-Active Compounds in Interaction of Bacteria with Interfaces.” Microbiol. Rev. 60(1):151-166 (1996).
Official Publication of the Association of American Feed Control Officials. AAFCO. Ingredient. Number 36.11 p 389 (2020).
Ohtsuka et al. (1985) J. Biol. Chem. 260:2605-2608.
Pearson (1994), Methods in Mol. Biol. 24:307-331.
Pearson (Proc. Nat. Acad. Sci. USA 85: 2444 (1988).
Perez, E. P., Festo, A. G., Co, K. G., and Norel, S. A. U.S. patent Ser. No. 12/085,841. “Method for Producing Calcium Soaps for Animal Feed.” (2009).
Polan, C. E., McNeill, J. J. and Tove, S. B. “Biohydrogenation of unsaturated fatty acids by rumen bacteria”. J. Bacteriol. 88(4):1056-1064 (1964).
Priest, F. G. “Extracellular Enzyme Synthesis in the Genus Bacillus.” Bacteriol. Rev. 41(3):711-753(1977).
Rossolini et al. (1994) Mol. Cell. Probes 8:91-98.
Rovers, M. “Improving fat digestibility with emulsifiers” All About Feed, October 2013.
Sambrook, J., Fritsch, E. F. and Maniatis, T. (2001) Molecular Cloning: A Laboratory Manual, 3rd Edition. Cold Spring Harbor Laboratory Press, Plainview, New York.
Smith and Waterman (Adv. Appl. Math. 2: 482 (1981).
Sonnhammer (1998), Nucleic Acids Res. 26:322-325.
Sumi, H. U.S. Pat. No. 6,677,143 B2. “Method for Culturing Bacillus subtilis natto to Produce Water-Soluble Vitamin K and Food Product, Beverage or Feed Containing the Cultured Microorganism or the Vitamin K Derivative.” (2004).
Tchuenbou-Maggaia, F. L., Norton, I. T., and Cox, P. W. “Hydrophobins stabilized air-filled emulsions for the food industry. Food Hydrocolloid. 23(7):1877-1885 (2009).
Tijssen (1993) Laboratory Techniques in Biochemistry and Molecular Biology—Hybridization with Nucleic Acid Probes, Part I, Chapter 2 (Elsevier, New York).
United States Pharmacopeia standards 61 (USP 41-NF 36 chapter 61. “Microbiological Examination of Nonsterile Products: Microbial Enumeration Tests.” (2018) United States Pharmacopeial Convention, Inc. ISBN 978-3-7692-7022-8
The United States Pharmacopeia and National Formulary Chapter 62 “Microbiological Examination of Nonsterile Products: Tests for Specified Microorganisms.” (2018)) United States Pharmacopeial Convention, Inc. ISBN 978-3-7692-7022-8
USP 41-NF 36 chapter<62>. “Microbiological Examination ofNonsterile Products: Tests for Specified Microorganisms.” (2018).
Wheeler, David L, Barret, T. Benson, D. Bryan, S., Canese, K. Chetvernin, V. Church, D. DiCuccio, M. Edgar, R. Federhen, S. Feolo, M. Geer, L., Helmberg, W. Kapustin, Y., Khovayko, O. Landsman, D., Lipman, D. Madden, T, Maglott, D. Miller, V. Ostell, J., Pruitt, K. Schuler, G. Shumway, M, Sequceira, E. Sherry, S. Sirotkin, K. Souvorov, A. Starchenko, G. Tatusov, R. Tatusova, T. Wagner, L. and Yaschenko, “Database resources of the national center for biotechnology information.” Nucleic Acidls Res. 36:D13-D21(2008).
Wu, Z., Ohajuruka, O. A., and Palmquist, D. L. “Ruminal synthesis, biohydrogenation, and digestibility of fatty acids by dairy cows. J. Dairy Sci. 74:3025-3034 (1991). Yadav, M. M. “Alkaline Protease Production by Isolated Bacillus sp in Submerged and Solid State Fermentation.” J. Bio. Innov. 2(4):161-167 (2013).
Zeng, B., Tan, Z., Zeng, J., Tang, S., Tan, C., Zhou, C., Han, X. and Zhong, R. “Effects of dietary non-ionic surfactant and forage to concentrate ratio on bacterial population and fatty acid composition of rumen bacteria and plasma of goats”. Anim. Feed Sci. Tech. 173:167-176 (2012).
Zerbino, D. R. and Birney, E. “Velvet: algorithms for de novo short read assembly using de Bruijn graphs.” Genome Res. 18.821-829 (2008).
Zhang, X., Jiang, C., Gao, Q., Wu, D., Tang, S., Tan, Z, and Han, X. “Effects of Dietary Alkyl Polyglycoside Supplementation on Lactation Performance, Blood Parameters, and Nutrient Digestibility in Dairy Cows”, Animals 9(8):549 (2019).

What is claimed is:

1. A surfactant and/or carbohydrate degrading composition, said composition comprising cultured plaque of Bacillus subtilis strain 6A-1 (6A-1), exudates or fractions of said 6A-1 or said cultured plaque, reference strain of said 6A-1 having been deposited at ATCC under deposit number PTA-125135.

2. The surfactant composition of claim 1, wherein said exudate comprises a non-polar extract of said 6A-1.

3. The composition of claim 1, wherein said composition comprises exudates that are free of cells and spores.

4. The surfactant composition of claim 1, comprising proteins CAB15086.1, SEQ ID NO: 2 or a sequence having at least 95% identity thereto or CAB15055.1 SEQ ID NO: 3 or a sequence having at least 95% identity thereto.

5. The composition of claim 1, comprising DNA sequence 6360-1, SEQ ID NO: 1 or a sequence having at least 95% identity thereto or a polypeptide comprising SEQ ID NO: 22 or a sequence having at least 95% identity thereto.

6. The surfactant composition of claim 1, wherein surfactant activity when quantified by methylene blue active substances of said composition is higher than a composition having surfactant activity produced by B. subtilis strain 168.

7. The surfactant composition of claim 1, wherein said surfactant when fed to an animal complexes unsaturated fatty acids fed to said animal with calcium fed to said animal.

8. The composition of claim 1, further comprising strain, cells or spores of said 6A-1.

9. The composition of claim 1, wherein said composition comprises exudates and said exudates are diluted, centrifuged, filtered, dried, extracted with a solvent, combined with an excipient, carrier or diluent, or a combination thereof.

10. The composition of claim 1, wherein said composition is produced by:
a) culturing said 6A-1 strain on solid-phase media;
b) producing cultured plaque;
c) scraping said cultured plaque; and
d) producing cultured plaque, exudates or fractions of said cultured plaque and producing said composition.

11. The carbohydrate degrading composition of claim 1, wherein said composition comprises CAB 15943.7, SEQ ID NO: 4 or a sequence having 95% identity thereto.

12. A method of producing a surfactant composition and/or carbohydrate degrading composition, said method comprising,
a) culturing on solid-phase media strain Bacillus subtilis 6A-1 (6A-1), reference culture comprising said 6A-1 having been deposited at ATCC under deposit number PTA-125135;
b) producing cultured plaque;
c) scraping said cultured plaque from said media; and
d) producing a surfactant and/or carbohydrate degrading composition comprising said cultured plaque, exudates or fractions of said cultured plaque, or a combination thereof.

13. The method of claim 12, further comprising,
a) diluting, centrifuging, filtering, drying or a combination thereof of said plaque or exudates;
b) extracting with a solvent a non-polar phase composition from said plaque or said exudate;
c) combining said plaque, exudate or fraction with an excipient, carrier or diluent;
d) separating exudate from cells; or
e) a combination of a)-d).

14. The method of claim 12, wherein said culture is maintained at a pH of 5.5 to 9.0.

15. The method of claim 12, wherein,
a) said media is wheat bran agar and is supplemented with salts of calcium; or
b) said media comprises liver-infusion tryptic soy agar (LITSA), and is supplemented with iron proteinate.

16. The method of claim 12, where said media comprising wheat bran agar is maintained at a pH of 7 and said media comprising LITSA media is maintained at a pH of 6-8.

17. A method of producing an animal or food produced by an animal having decreased saturated fatty acid composition and/or increased unsaturated fatty acid composition, or having increased calcium absorption and/or retention, the method comprising feeding said animal a composition comprising,
(i) the composition of claim 1;
(ii) proteins CAB15086.1, SEQ ID NO: 2 and CAB15055.1, SEQ ID NO: 3; or
(iii) proteins CAB 15943.7, SEQ ID NO: 4 or a sequence having 95% identity thereto.

18. An animal feed additive comprising cultured plaque, exudate or, fraction produced by strain Bacillus subtilis 6A-1 (6A-1), reference culture comprising said 6A-1 having been deposited at ATCC under deposit number PTA-125135.

19. A feed additive for an animal, the additive comprising proteins CAB15086.1, SEQ ID NO:
2 and CAB15055.1, SEQ ID NO: 3.

20. The additive of claim 19, said additive produced by strain Bacillus subtilis 6A-1 (6A-1), reference culture comprising said 6A-1 having been deposited at ATCC under deposit number PTA-125135.

21. The additive of claim 19, wherein said composition comprises a composition selected from SEQ ID NO: 1 or a polypeptide comprising SEQ ID NO: 22 or a sequence having at least 95% identity thereto, strain, cells, spores, cultured plaque, exudates or fractions of strain Bacillus subtilis 6A-1 (6A-1), reference culture comprising said 6A-1 having been deposited at ATCC under deposit number PTA-125135.

22. A method of identifying strain Bacillus subtilis 6A-1 (6A-1), reference culture comprising said 6A-1 having been deposited at ATCC under deposit number PTA-125135, or cell, spore or composition produced by said 6A-1 strain, cell or spore, the method comprising detection of the a region specific to nucleic acid sequence 6360-1, SEQ ID NO: 1.

23. The method of claim 22, wherein said method comprises amplifying a DNA fragment of 17 or more consecutive nucleotides of said sequence, or hybridizing a nucleic acid of a sample with a specific probe for said sequence.

24. An animal feed additive comprising plaque, exudate or fraction produced from strain Bacillus subtilis 6A-1 (6A-1), reference culture comprising said 6A-1 having been deposited at ATCC under deposit number PTA-125135.

25. An animal, meat, milk, egg or food product from an animal having been fed the composition of claim 1, said animal, meat, milk, egg or food product having decreased saturated fatty acid composition and/or increased unsaturated fatty acid composition, or having increased calcium absorption and/or retention.

26. A surfactant composition comprising proteins CAB15086.1, SEQ ID NO: 2 or a sequence having at least 95% identity thereto or CAB15055.1 SEQ ID NO: 3 or a sequence having at least 95% identity thereto.

27. The surfactant composition of claim 26, wherein said surfactant when fed to an animal complexes unsaturated fatty acids fed to said animal with calcium fed to said animal.
